SEQUENCE LISTING (10 sequences)

SEQ ID NO: 1 - DNA, 35 bp, Artificial Sequence
  Synthetic polynucleotide
actgaattcgccaccatgtggcccggaatattggt

SEQ ID NO: 2 - DNA, 29 bp, Artificial Sequence
  Synthetic polynucleotide
atcggatcctctcaggtgctagctgggac

SEQ ID NO: 3 - DNA, 38 bp, Artificial Sequence
  Synthetic polynucleotide
tagagctagcgaattgccaccatgtggcccggaatatt

SEQ ID NO: 4 - DNA, 35 bp, Artificial Sequence
  Synthetic polynucleotide
cggcggccgcggatctcaggtgctagctgggacag

SEQ ID NO: 5 - DNA, 32 bp, Artificial Sequence
  Synthetic polynucleotide
gggccaagccacagccattgccatccaggcag

SEQ ID NO: 6 - DNA, 32 bp, Artificial Sequence
  Synthetic polynucleotide
ctgcctggatggcaatggctgtggcttggccc

SEQ ID NO: 7 - DNA, 20 bp, Artificial Sequence
  Synthetic polynucleotide
ggctcatcctcaccgtcctg

SEQ ID NO: 8 - DNA, 20 bp, Artificial Sequence
  Synthetic polynucleotide
gatgtactgcatcgtgtcgt

SEQ ID NO: 9 - protein, 40 aa, Staphylococcus aureus
QIDDNVANSIVSQLLFLQAQDSEKDIYLYINSPGGSVTAG

SEQ ID NO: 10 - protein, 42 aa, Homo sapiens
PIDDSVASLVIAQLLFLQSESNKKPIHMETYINSPGGVVTAG

(19) United States
(12) Patent Application Publication
(10) Pub. No.: US 2022/0143024 A1
(43) Pub. Date: May 12, 2022

(54) METHODS OF USING IMIPRIDONES

(71) Applicants: Board of Regents, The University of Texas System, Austin, TX (US); University Health Network, Toronto (CA)

(72) Inventors: Michael ANDREEFF, Houston, TX (US); Jo ISHIZAWA, Houston, TX (US); David SCHIMMER, Toronto (CA); Sara ZARABI, Toronto (CA)

(73) Assignees: Board of Regents, The University of Texas System, Austin, TX (US); University Health Network, Toronto, ON (CA)

(21) Appl. No.: 17/432,633

(22) PCT Filed: Feb. 21, 2020
(86) PCT No.: PCT/US20/19142

§ 371 Date: Aug. 20, 2021

Related U.S. Application Data
(60) Provisional application No. 62/809,140, filed on Feb. 22, 2019.
(60) Provisional application No. 62/908,105, filed on Sep. 30, 2019.

(51) Int. Cl. A61K 31/519; A61K 31/635; A61P 35/00

(52) U.S. Cl. CPC A61K 31/519; A61P 35/00; A61K 31/635

(57) ABSTRACT

Provided herein are methods of using ClpP levels and mutation status as a marker for the selection and treatment of cancer patients who will respond to the administration of imipridones. Also provided are methods of treating patients having Perrault syndrome. Also provided are methods of killing bacterial cells and treating bacterial infections using imipridones.

### REFERENCE TO RELATED APPLICATIONS

The present application claims the priority benefit of U.S. provisional application No. 62/908,105, filed Sep. 30, 2019, and U.S. provisional application No. 62/809,140, filed Feb. 22, 2019, the entire contents of each of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Jan. 29, 2020, is named UTFC1440WO_ST25.txt and is 2.8 kilobytes in size.

### BACKGROUND

### 1. Field

The present invention relates generally to the fields of medicine and oncology. More particularly, it concerns methods for selecting patients for treatment with imipridones as well as treating patients so selected.

### 2. Description of Related Art

Despite newly developed targeted agents, the majority of hematologic malignancies and solid tumors are incurable. This includes essentially all patients with TP53 mutations. Accumulating evidence demonstrates that mitochondrial function is critical for maintenance and therapy-resistance of leukemias (Cole et al., 2015; Farge et al., 2017; Kuntz et al., 2017; Moschoi et al., 2016; Samudio et al., 2010; Skrtic et al., 2011) and certain solid tumors (Birsoy et al., 2014; Kotschy et al., 2016; Viale et al., 2014), and therapeutic strategies to effectively disrupt the integrity of mitochondria have been investigated (Birsoy et al., 2014; Cole et al., 2015; Konopleva et al., 2006; Konopleva et al., 2016; Kotschy et al., 2016; Kuntz et al., 2017; Pan et al., 2014; Pan et al., 2017; Skrtic et al., 2011; Viale et al., 2014). Nevertheless, anti-tumor agents that can disrupt mitochondrial structure and function are.

### SUMMARY

As such, provided herein are methods of treating cancer patients by disrupting mitochondrial structure and function. Such methods comprise administering an imipridone to a patient having cancer. Also provided herein are methods to predict whether a patient will be sensitive to the anti-cancer activity of imipridones based on the level of the mitochondrial protease ClpP.

In one embodiment, provided herein are methods of selecting a patient having a cancer for treatment with an agent that activates mitochondrial proteolysis, the methods comprising (a) determining a ClpP level in the cancer, and (b) selecting the patient for treatment if the ClpP level in the cancer is higher than a reference level. In some aspects, the reference level is a level that is one standard deviation below an average ClpP level in a healthy population. In some aspects, the methods further comprise administering an effective amount of an agent that activates mitochondrial proteolysis.

In some aspects, the agent that activates mitochondrial proteolysis is a ClpP activating agent. In certain aspects, the ClpP activating agent is an imipridone. In certain aspects, the imipridone is ONC201, ONC206, ONC212, or ONC213.

In one embodiment, provided herein are methods of treating a patient having a cancer, the methods comprising administering a therapeutically effective amount of an agent that activates mitochondrial proteolysis to the patient, wherein the patient's cancer has a ClpP level that is higher than a reference level. In some aspects, the reference level is a level that is one standard deviation below an average ClpP level in a healthy population.

In one embodiment, provided herein are methods of treating a patient having a cancer, the method comprising: (a) detecting whether the patient's cancer has a ClpP level that is higher than a reference level by: (i) obtaining or having obtained a biological sample from the cancer; and (ii) performing or having performed an assay on the biological sample to determine a ClpP level; (b) selecting or having selected the patient for treatment when the cancer has a ClpP level that is higher than a reference level; and (c) administering or having administered to the selected patient a therapeutically effective amount of an agent that activates mitochondrial proteolysis. In some aspects, the reference level is a level that is one standard deviation below an average ClpP level in a healthy population.

In some aspects, the ClpP level in the cancer is determined by western blot, ELISA, immunoassay, radioimmunoassay, or mass spectrometry. In some aspects, the methods further comprise administering at least a second anti-cancer therapy to the patient. In certain aspects, the second anti-cancer therapy is a surgical therapy, chemotherapy, radiation therapy, cryotherapy, hormonal therapy, toxin therapy, immunotherapy, or cytokine therapy. In certain aspects, the chemotherapy is venetoclax. In certain aspects, the immunotherapy is an immune checkpoint inhibitor.

In some aspects, the methods further comprise reporting the ClpP level. In certain aspects, the reporting comprises preparing a written or electronic report. In certain aspects, the methods further comprise providing the report to the subject, a doctor, a hospital, or an insurance company.

In one embodiment, provided herein are methods of selecting a patient having a cancer for treatment with an agent that activates mitochondrial proteolysis, the method comprising (a) determining a ClpP protein mutation status in the cancer, and (b) selecting the patient for treatment if the cancer has a D190A mutation in the ClpP protein. In some aspects, the methods further comprise administering an effective amount of an agent that activates mitochondrial proteolysis. In certain aspects, the agent that activates mitochondrial proteolysis is a ClpP activating agent. In certain aspects, the ClpP activating agent is an imipridone. In certain aspects, the imipridone is ONC201, ONC206, ONC212, or ONC213.

In one embodiment, provided herein are methods of treating a patient having a cancer, the method comprising administering a therapeutically effective amount of an agent that activates mitochondrial proteolysis to the patient, wherein the patient's cancer has a D190A mutation in a ClpP protein.

In one embodiment, provided herein are methods of treating a patient having a cancer, the method comprising: (a) detecting whether the patient's cancer has a D190A mutation in a ClpP protein by: (i) obtaining or having obtained a biological sample from the cancer; and (ii) performing or having performed an assay on the biological sample to determine whether the patient's cancer has a D190A mutation in a ClpP protein; (b) selecting or having selected the patient for treatment when the cancer has a D190A mutation in the ClpP protein; and (c) administering or having administered to the selected patient a therapeutically effective amount of an agent that activates mitochondrial proteolysis.

In some aspects, the D190A mutation in the ClpP protein is detected by western blot, ELISA, mass spectrometry, or sequencing a nucleic acid encoding ClpP. In certain aspects, the western blot or ELISA are performed using an antibody that specifically detects ClpP having the D190A mutation. In certain aspects, the nucleic acid is an mRNA encoding ClpP. In certain aspects, the nucleic acid is genomic DNA encoding ClpP.

In some aspects, the agent that activates mitochondrial proteolysis is a ClpP activating agent. In certain aspects, the ClpP activating agent is an imipridone. In certain aspects, the imipridone is ONC201, ONC206, or ONC212.

In some aspects, the methods further comprise administering at least a second anti-cancer therapy to the patient. In certain aspects, the second anti-cancer therapy is a surgical therapy, chemotherapy, radiation therapy, cryotherapy, hormonal therapy, toxin therapy, immunotherapy, or cytokine therapy. In certain aspects, the chemotherapy is venetoclax. In certain aspects, the immunotherapy is an immune checkpoint inhibitor.

In some aspects, the methods further comprise reporting the ClpP D190A mutation status. In certain aspects, the reporting comprises preparing a written or electronic report. In certain aspects, the methods further comprise providing the report to the subject, a doctor, a hospital, or an insurance company.

In some aspects, the patient is in remission and the method prevents relapse. In some aspects, the methods eliminate chemo-resistant cells. In some aspects, the cancer is AML. In some aspects, the patient has previously undergone at least one round of anti-cancer therapy. In some aspects, the patient is a human.

In one embodiment, provided herein are methods of killing bacterial cells, the method comprising contacting the bacterial cells with a lethal amount an imipridone. In certain aspects, the imipridone is ONC201, ONC206, or ONC212. In some aspects, the bacterium is a gram-positive bacterium. In some aspects, the bacterium is selected from the group consisting of Staphylococcus, Streptococcus, Enterococcus, Clostridium, Corynebacterium, and Peptostreptococcus. In some aspects, the bacterium is Staphylococcus.

In one embodiment, provided herein are methods of treating a bacterial infection in a subject in need thereof, the method comprising administering to the subject a therapeutically effective amount of an imipridone. In certain aspects, the imipridone is ONC201, ONC206, or ONC212. In some aspects, the bacteria are antibiotic resistant. In some aspects, the bacterium is a gram-positive bacterium. In some aspects, the bacterium is selected from the group consisting of Staphylococcus, Streptococcus, Enterococcus, Clostridium, Corynebacterium, and Peptostreptococcus. In some aspects, the bacterium is Staphylococcus.

In one embodiment, provided herein are methods of treating a patient having Perrault syndrome, the methods comprising administering or having administered to the patient a therapeutically effective amount of an agent that activates mitochondrial proteolysis. In some aspects, the agent that activates mitochondrial proteolysis is a ClpP activating agent. In some aspects, the ClpP activating agent is an imipridone. In some aspects, the imipridone is ONC201, ONC206, ONC212, or ONC213. In some aspects, the patient has a mutation in CLPP or HSD17B4. In some aspects, the methods improve the patient's hearing, prevent further hearing loss in the patient, and/or prevent hearing loss from occurring in the patient. In some aspects, the patient is female and the methods improve ovarian function in the patient, prevent further ovarian dysgenesis in the patient, and/or prevent ovarian dysgenesis from occurring in the patient.

In one embodiment, provided herein is the use of an agent that activates mitochondrial proteolysis, such as, for example, an imipridone, in the manufacture of a medicament for treating a patient having a cancer with a D190A mutation in their ClpP gene or a cancer that expresses a high level of ClpP. In one embodiment, provided herein is an agent that activates mitochondrial proteolysis, such as, for example, an imipridone, for use in treating a patient having a cancer with a D190A mutation in their ClpP gene or a cancer that expresses a high level of ClpP.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. Mitochondrial ClpP activation induces anti-tumor effects in vitro and in vivo. (FIG. 1A) Tetracycline-inducible over-expression of wild-type or constitutively active Y118A mutant ClpP in OCI-AML3 and Z138 cells. Cells were treated with tetracycline at indicated concentrations for 144 hours. Data represent percent mean±SD apoptotic (annexin V-positive) cells (top). ***P<0.001, ****P<0.0001. ClpP protein levels were examined by immunoblot analysis (bottom). (FIG. 1B) Survivals of xenograft mice using Z138 cells with tetracycline-inducible Y118A mutant ClpP over-expression. The mice (n=10 each) were treated with or without tetracycline (2 mg/mL in drinking water). The “Tetracycline” survival curve is the one that intersects the x-axis at about 51 days. (FIG. 1C) Effects of ADEP1 on degradation of FITC-casein by recombinant WT ClpP. Mean±SD. (FIG. 1D) Effects of ADEP1 on viability of OCI-AML2 cells measured by alamar blue assay after a 72-hour period of exposure to the drug. Mean±SD.

FIGS. 2A-E. The imipridones ONC201 and ONC212 activate mitochondrial ClpP. (FIG. 2A) A chemical library of 747 molecules was screened for their effects on degradation rate of fluorogenic substrate FITC-casein by recombinant WT ClpP. (FIG. 2B) Chemical structures of ONC201 and ONC212. (FIGS. 2C & 2D) Effects of ONC201 and ONC212 on degradation of fluorogenic substrates (AC-WLA-AMC (FIG. 2C) and FITC-casein (FIG. 2D) by recombinant WT ClpP. Mean±SD. (FIG. 2E) Degradation of α-casein by purified recombinant WT ClpP and ClpXP complexes treated for 3 h with ONC201, ONC212, or vehicle control (DMSO) in FITC-casein assay buffer detected on SDS-PAGE.

FIGS. 3A-H. ONC201 binds to ClpP and is cytotoxic to leukemia and lymphoma cells. (FIG. 3A) Isothermal calorimetry binding experiment showed nonstandard behavior when 100 μM ONC201 was titrated into 20 μM ClpP (concentration of ClpP monomer). (FIG. 3B) ONC201 binds in the hydrophobic pocket between two subunits (left, hydrogen bonds are indicated by dashed lines; water molecule mediating hydrogen bonding in red sphere). (FIG. 3C) Binding of ONC201 to ClpP opens up the axial pore and induces protein compaction (top and front view; apo-grey PDB ID:1TG6). Bottom row: ONC201 binding increases dynamics of the N-termini (pore region) and the heptamer interface as evidenced by temperature factor variation (B-factors). (FIG. 3D) ONC201 binding to ClpP induces pores in the heptamer interface (cross-section through the assembled ClpP tetradecamer; position of pores indicated by black triangles). Closeup of the pore (inset) between chains C (bottom left), D (bottom right) and symmetry-related chain K (top). Protein chains are indicated by ribbon colored based on residue B-factors (protein surface in shades of gray). (FIG. 3E) Model of ONC212-binding to ClpP. ONC212 clamps into two surface depressions at the interface of two human ClpP subunits. The trifluoromethyl substituent extends deeply and fits well into the pocket that in the crystal structure of the ONC201 complex accommodates its 4-(2-methylbenzyl) group. The ligand is displayed as sticks and the surrounding protein is shown in surface representation. (FIG. 3F) Concentration-dependent effects of treatment with ONC201 (I) and ONC212 (II) on thermal stability of endogenous ClpP in OCI-AML2 cells assessed using cellular thermal shift assays (CETSA). UHC: unheated control. OCI-AML2 cells were treated with increasing concentrations of ONC201 or ONC212 for 30 minutes, washed and re-suspended in PBS containing proteinase inhibitors, and heated to 67° C. for 3 minutes prior to collection of cell lysates for immunoblotting. (III) Effect of removal of ONC201 from media on thermal stability of endogenous ClpP in intact OCI-AML2 cells. ONC201 (10 μM) treated cells were washed with PBS and re-incubated in fresh medium for up to 75 min prior to CETSA. w=wash. (FIG. 3G) Effects of ONC201 and ONC212 on viability of OCI-AML2, TEX, OCI-AML3, and Z138 cells. Data represent percent mean±SD viable or apoptotic cells measured by alamar blue assay in OCI-AML2, TEX cells, or by annexin V assay in OCI-AML3 and Z138 cells after a 72-hour period of exposure to the drugs. (FIG. 3H) Changes in live cell number by ONC201 and ONC212 compared to untreated controls in primary AML and normal bone marrow mononuclear cells (BM-MNC). Cells were treated with ONC201 and ONC212 at indicated concentrations for 72 hours. Annexin V- and DAPI-negative cells were measured by flow cytometry and normalized to that in untreated controls. #, ##: samples which were relatively resistant to ONC201 (specified in Table 3).

FIGS. 4A-H. Cytotoxicity of imipridones is ClpP-dependent. (FIG. 4A) Effects of ONC201 and ONC212 on viability in ClpP+/+ & ClpP−/− T-REx HEK293 cells. Data represent percent mean±SD viable cells measured by alamar blue assay after a 72-hour period of exposure to the drugs. (FIG. 4B) Correlation between pretreatment expression level of ClpP and the effects of ONC201 on viability of primary AML samples measured by annexin V assay after a 72-hour period of exposure to the drug. ClpP levels were quantified by immunoblot analysis of untreated samples. Low ClpP=samples with ClpP levels that were 1 SD below average. High ClpP=all other samples. (FIG. 4C) Effects of wild-type and D190A-ClpP on degradation of fluorogenic AC-WLA-AMC. Mean±SD. (FIG. 4D) Effects of ONC201 and ONC212 on degradation of fluorogenic substrates (AC-WLA-AMC) (left) and FITC-casein (right)) by D190A ClpP. Mean±SD. (FIG. 4E) ITC data for ONC201 (100 μM) titrated into D190A-ClpP (20 μM; concentration of ClpP monomer). (FIG. 4F) The location of D190 and R226 at the interface of two heptamer rings in an apparently closed conformation of human mitochondrial ClpP. (FIG. 4G) Overexpression (O/E) of wild-type ClpP in ONC201-resistant (ONC-R) Z138 cells carrying D190A mutant ClpP. Cells were treated with ONC201 and ONC212 at indicated concentrations for 72 hours. Data represent percent mean±SD apoptotic (annexin V-positive) cells. E/V; empty vector as control. Protein expression levels of ClpP was assessed by immunoblotting. **P<0.01, ***P<0.001, ****P<0.0001. (FIG. 4H) Overexpression of wild-type or D190A-ClpP in parental (ONC201-sensitive) Z138 and OCI-AML3 cells. Cells were treated with ONC201 and ONC212 at indicated concentrations for 72 hours. Data represent percent mean±SD apoptotic (annexin V-positive) cells. Protein expression levels of ClpP were assessed by immunoblotting. **P<0.01, ***P<0.001, ****P<0.0001.

FIGS. 5A-E. ClpP hyperactivation induces apoptosis following reduction of respiratory chain complex subunits. (FIG. 5A) A subset of ClpP mitochondrial interactors was identified using BioID-MS and categorized according to selected gene ontology biological processes. Decreases in spectral counts following ONC201 treatment is illustrated and proportional to the decreases in color intensity. (FIGS. 5B-E) Immunoblot analysis of respiratory chain complex subunits in parental (ONC-sensitive) Z138 cells and ONC201-resistant Z138 cells (the single-clone #2 carrying D190A mutant ClpP) with over-expression of wild-type ClpP or an empty vector (FIG. 5B); in parental (ONC-naive) Z138 cells with over-expression of wild-type ClpP, D190A mutant ClpP, or an empty vector (FIG. 5C); in tetracycline-inducible Y118A mutant ClpP in Z138 cells (FIG. 5D); in primary AML cells and normal bone marrow (NBM) cells (FIG. 5E). AML #3_1 and #3_2 are from the same patient but at different time points in relapse. Cells were treated with ONC201 at indicated concentrations for 24 hours.

FIGS. 6A-E. ClpP hyperactivation by ONC201 impairs oxidative phosphorylation. (FIG. 6A) Effect of ONC201 on oxygen consumption in Z138 and Z138 D190A ClpP cells (measured by Seahorse Analyzer). 2 μM Oligomycin and 0.25 μM FCCP were used to derive parameters of mitochondrial respiration. (FIG. 6B) Effects of ONC201 treatment on activity of respiratory chain complexes I, II, & IV in OCI-AML2 cells. (FIG. 6C) Effect of ONC201 treatment on mitochondrial ROS production in Z138 and Z138 D190A ClpP cells. Percent mean±SD from one of 3 representative experiment is shown. (FIG. 6D) Effect of ONC201 treatment on mitochondrial morphology. Mitochondria were imaged by a transmission electron microscopy in OCI-AML3 cells treated with or without 5 mM ONC201 for 24 hours. (FIG. 6E) Immunoblot of ATF4, p-eIF2α, and eIF2α in Y118A ClpP-overexpressed Z138 cells. Z138 cells with tetracycline-inducible Y118A ClpP were treated with tetracycline for 48 hours at the indicated concentrations.

FIGS. 7A-E. ClpP activation exerts anti-tumor effects in vivo. (FIG. 7A) Tumor burden measured by luciferase activity using IVIS imaging in xenograft mice with wild-type or D190A-mutant ClpP overexpressing Z138 cells treated with or without ONC212. Mice (n=7 each) were treated with ONC212 (50 mg/kg every other day, oral gavage) or vehicle after confirming engraftment. (FIG. 7B) Intensities of luminescence detected by IVIS imaging in the mice in FIG. 6A. (FIG. 7C) Survivals of xenograft mice using Z138 cells over-expressed with WT or D190A ClpP. ONC212 increased survival. (FIG. 7D) Tumor volumes of xenograft mice using OCI-AML2 cells. Mice (n=10 each) were treated with ONC201 (100 mg/kg twice daily, oral gavage) or vehicle from 5 days after transplantation for 13 days. (FIG. 7E) Survivals of Pdx AML mice. Pdx cells [t(9;11)(p22; q23), CEBPA, and ATM mutants] were treated with or without 250 nM ONC212 for 36 hours, then injected into NSG mice (n=10 each). ONC212 increased survival.

FIGS. 8A-C. An activating mutation Y118A in ClpP and imipridones hyperactivate recombinant WT ClpP in vitro. (FIG. 8A) Sequence alignment of S. aureus (SEQ ID NO: 9) and human ClpP (SEQ ID NO: 10). (FIG. 8B) FITC-casein degradation kinetics of WT ClpP and Y118A ClpP mutants. (FIG. 8C) Effects of ONC201 and ONC212 on degradation of fluorogenic substrates (AC-WLA-AMC and FITC-casein) by WT ClpP. Error bars represent mean±SD for triplicate experiments.

FIGS. 9A-C. ClpP activated by imipridones degrades ClpP substrates while retaining its specificity in vitro. (FIG. 9A) Effects of ONC201, ONC212, ADEP1, and ONC201 inactive isomer on degradation of fluorogenic substrates (Phe-hArg-Leu-ACC, Clptide, and MCA-Pro-Leu-Gly-Pro-Lys (DNP)-OH) by WT ClpP. Error bars represent mean±SD for triplicate experiments. (FIG. 9B) Effects of ONC201 inactive isomer on degradation of FITC-casein (left) and Ac-WLA-AMC (right) by recombinant WT ClpP. Mean±SD. (FIG. 9C) Effect of pre-incubation of WT ClpP with ONC201 (0-60 min) on degradation rate of FITC-casein. Mean±SD.

FIGS. 10A-F. ONC201 & ONC212 hyperactivate recombinant WT ClpP in vitro. (FIG. 10A) Binding of ClpP to ONC201 measured by isothermal calorimetry. 500 μM WT ClpP titrated into 50 μM ONC201. (FIG. 10B) Control—buffer titrated into 50 μM drug. (FIG. 10C) Gel filtration showed shift to higher molecular weight species when ClpP was run with ONC201 (1:1). Black=14-mer; Gray=7-mer. (FIG. 10D) ONC201 binds in the hydrophobic pocket between two subunits (hydrogen bonds are indicated by dashed lines; water molecule mediating hydrogen bonding in sphere). (FIG. 10E) ONC201 fits well into the positive mFo-DFc difference density. Map calculated by omitting ONC201 molecules from the structure and contoured at 3a. (FIG. 10F) Catalytic triad rearranges itself upon ONC201 binding to ClpP—both His178 and Asp227 move away from Ser153 (apo—grey; ONC201 bound—violet).

FIGS. 11A-C. ONC201 binds to wild-type ClpP in OCI-AML2 cells and induces apoptosis in cancer cells. (FIG. 11A) Effect of treatment with 10 μM ONC201 on thermal stability of endogenous ClpP in OCI-AML2 cells tested by CETSA. U: untreated control; T: treated with 10 μM ONC201. Intact cells were treated with ONC201 for 30 min and heated (59-67° C.) for 3 min prior to collection of cell lysates for immunoblotting. (FIG. 111B) Effects of ONC212 on viability of HCT-116, HeLa, OC316, and SUM159 cells. Data represent percent mean±SD viable cells measured by annexin V assay after a 72-hour period of exposure to ONC212. (FIG. 11C) Apoptosis in Z138 and OCI-AML3 cells treated with ONC201 and ONC212. Cells were treated with ONC201 or ONC212 at indicated concentrations for 72 or 120 hours. Annexin V- and PI-negative cells were counted as live cells (upper panels), and Annexin V+ cells were counted as apoptotic cells (lower panels), normalized to untreated samples.

FIGS. 12A-C. Cytotoxicity of imipridones is ClpP-dependent. (FIGS. 12A-B) Effects of ONC201 and its inactive isomer on viability in ClpP+/+ or ClpP−/− T-REx HEK293 (FIG. 12A) and ONC201-sensitive or ONC201-resistant Z138 (FIG. 12B) cells. Data represent percent mean±SD viable cells measured by alamar blue assay after a 72-hour period of exposure to the compounds. (FIG. 12C) Effect of ONC201 on viability of primary AML samples measured by annexin V assay after a 72-hour period of exposure. ClpP expression level in each sample was measured by immunoblot analysis of untreated samples.

FIGS. 13A-E. ONC201-resistant single-cell clones were resistant to ONC201 and ONC212 and harbored a heterozygous D190A mutation. (FIG. 13A) Sensitivity of ONC201-naïve and ONC201-resistant Z138 to ONC201 and ONC212 was assessed by Annexin V assays. Data represent percent mean±SD viable (annexin V and PI double negative) cells. The resistant cells were less sensitive. (FIG. 13B) Sensitivity of ONC201-resistant cells to standard chemo-agents. ONC201-resistant Z138 cells (clone #2) were treated with Adriamycin (upper panels) and Vincristine (lower panels) at indicated concentrations for 72 hours. Annexin V-positive cells (left) and Annexin V/PI double negative cells (right) were measured by flow cytometry. (FIG. 13C) Result of RNA sequencing of parental (ONC-sensitive) and ONC-resistant Z138 cells. Individual reads are visualized below for each cell line, and above bar graphs indicate the number of reads (“pileup”) at each nucleotide of the genomic exon sequence. Arrows indicate the position of wild-type A569 and A569C mutation. (FIG. 13D) Sensitivity of single cell clones of ONC201-resistant Z138 cells to ONC201. Single cell clones derived from ONC201-resistant Z138 cells were treated with ONC201 at indicated concentrations for 72 hours. Apoptotic cells (annexin V-positive) cells (upper) and live (Annexin V- and PI-double negative) cells (lower) were measured by flow cytometry. (FIG. 13E) Sensitivity of single-cell clones #2 and #4 derived from ONC201-resistant Z138 cells to ONC201 and ONC212 was assessed by Annexin V assays. Data represent percent mean±SD viable (annexin V and PI double negative) cells.

FIGS. 14A-E. D190A mutation in ClpP renders tumor cells resistant to imipridones. (FIG. 14A) Sanger sequence of genomic DNA, related to FIG. 4B. A D190A heterozygous mutation was detected in all the tested seven single-cell clones. (FIG. 14B) The location of D190 and Asp227 in the 3-D structure of an apparently closed conformation of human mitochondrial ClpP. D227 (Asp227) is 6.4 angstroms away from D190 and part of the catalytic triad of ClpP. (FIGS. 14C-D) Changes in live cell number by ONC201 and ONC212 on ClpP-overexpressed Z138 and OCI-AML3 cells. Viable cells were measured by flow cytometry. Data represent percent mean±SD viable (annexin V and PI double negative) cells. (FIG. 14C) WT ClpP over-expressing ONC201-resistant Z138 cells. (FIG. 14D) WT or D190A ClpP over-expressing OCI-AML3 and Z138 cells. *P<0.05, **P<0.01, ***P<0.001, ****P<0.0001. (FIG. 14E) Overexpression of D190A-ClpP in HCT116 cells. Cells were treated with ONC201 and ONC212 at indicated concentrations for 72 hours. Data represent percent mean±SD apoptotic (annexin V-positive) cells. Protein expression levels of ClpP were assessed by immunoblotting. EV; empty vector, OE; overexpression. #; invisible bars because of low numerical values. ***P<0.001, ****P<0.0001.

FIGS. 15A-D. ClpP hyperactivation induces apoptosis following reduction of respiratory chain complex subunits. (FIG. 15A) Immunoblot of SDHA, SDHB, and NDUFA12 in OCI-AML3 cells treated with ONC212 for 24 hours at indicated concentrations. (FIG. 15B) Immunoblot of respiratory chain complex subunits. OCI-AML3 cells were treated with ONC201 or ONC212 at indicated concentrations for 24 hours. (FIG. 15C) Immunoblot of SDHB and NDUFA12 in HCT-116, HeLa, OC316, and SUM159 cells treated with ONC212 for 24 hours at indicated concentrations. (FIG. 15D) Immunoblot analysis of respiratory chain complex subunits in HCT116 cells with over-expression of D190A mutant ClpP or an empty vector (EV).

FIGS. 16A-B. Reduction of respiratory chain complex subunits by imipridones is not transcriptionally but by activation of protein degradation in mitochondria. (FIG. 16A) Effect of ONC201 (0.6 μM) on levels of mRNA encoding mitochondrial respiratory chain subunits in OCI-AML2 and Z138 cells. (FIG. 16B) Immunoblots of citrate synthase (CS), UQCRC2 (complex III), and NDUFB8 (complex I) in mitochondrial lysates isolated from ClpP−/− HEK293T-REx and OCI-AML2 cells treated with increasing concentrations of ONC201 with or without recombinant ClpP (6 μM) after a brief (3 h) period of incubation.

FIG. 17. Early clinical response of ONC201 in an AML patient. A patient with AML refractory to decitabine, fludarabine, cyarabine and two investigational IDH2 inhibitors was enrolled in the Phase 1 trial of ONC201. Blasts (50% to 3%) and platelet transfusion requirements were reduced after oral administration of a single dose of ONC201 (250 mg). Arrows indicate ONC201 administration.

FIG. 18. Genetic activation of ClpP sensitizes leukemia and lymphoma cells to venetoclax (ABT-199). Constitutively active ClpP mutant (Y118A), with the tetracycline-inducible system, was transfected by lentivirus into OCI-AML3 and Z138 cells. Cells were treated with tetracycline, which induces Y118A ClpP mutant in a tetracycline dose-dependent manner by 72 hrs, and subsequently exposed to venetoclax (ABT-199) in indicated concentrations. Following treatment, cells were assessed for AnnexinV staining.

FIG. 19. Responders in ONC201 clinical trials showed ClpP-positive leukemia cells, while a non-responder was negative for ClpP. Pre-treatment bone marrow biopsy samples from 11 patients among the 30 enrolled patients were obtained and stained for ClpP. Representative micrographs are shown.

### DETAILED DESCRIPTION

The mitochondrial caseinolytic protease P (ClpP) plays a central role in mitochondrial protein quality control by degrading misfolded proteins. Using genetic and chemical approaches, it was shown that hyperactivation of the protease selectively kills cancer cells, independently of p53 status, by selective degradation of its respiratory chain protein substrates and disrupts mitochondrial structure and function, while it does not affect non-malignant cells. Antineoplastic compounds-imipridones-were identified as potent hyperactivators of ClpP. Through biochemical studies and crystallography, it was shown that imipridones bind ClpP non-covalently and induce proteolysis by diverse structural changes. These findings suggest a general concept of inducing cancer cell lethality through activation of mitochondrial proteolysis. In addition, patients with the lowest levels of ClpP are less sensitive to ClpP hyperactivation. Thus, ClpP levels and/or ClpP mutation status can be used to select patients likely to respond to treatment with imipridones.

### I. CLPP

Eukaryotic cells have two separate genomes; nuclear DNA and mitochondrial DNA. Mitochondrial DNA encodes two rRNAs, 22 t-RNAs, and 13 of the 90 proteins in the mitochondrial respiratory chain. The remaining mitochondrial proteins are encoded by nuclear genes, translated in the cytoplasm and imported into the mitochondria. Mitochondria possess their own protein synthesis apparatus including mitochondrial ribosomes, initiation factors, and elongation factors. In addition, mitochondria have protein degradation complexes that regulate their protein levels by eliminating excess and/or damaged proteins. To date, at least 15 proteases have been identified in different mitochondrial compartments, including caseinolytic protease P (ClpP), which is located in the mitochondrial matrix. ClpP is an oligomeric serine protease that is similar to the cytoplasmic/nuclear proteasome (Corydon et al., 1998).

After import into the mitochondria, ClpP is assembled into a double-ringed tetradecameric structure with a hollow chamber containing proteolytic active sites. The tetradecameric structure is capped at each end by an AAA+ATPase chaperone, ClpX (de Sagarra et al., 1999). The function of the ClpXP complex in mitochondria is not fully understood, but insights have been gained from its bacterial homologue that shares structural homology. Bacteria lack a ubiquitin-dependent proteolytic system and instead eliminate intracellular proteins with a family of proteases including the bacterial ClpXP complex. In bacteria, ClpX recognizes and unfolds native substrates and feeds them into the barrel of the ClpP protease for degradation.

The bacterial ClpXP complex is responsible for degrading excess proteins including those whose translation stalls on ribosomes. Recently, it was demonstrated that mitochondrial ClpP is over-expressed in 45% of primary AML samples (Cole et al., 2015). ClpP is equally expressed in stem cell and bulk populations, and over-expression occurs across the spectrum of cytogenetic and molecular mutations. ClpP expression is positively correlated with expression of genes related to the mitochondrial unfolded protein response (Cole et al., 2015). Functionally, ClpP maintains the integrity of oxidative phosphorylation as inhibition of the protease results in the accumulation of misfolded or degraded respiratory chain complex subunits and respiratory chain dysfunction in AML cells (Cole et al., 2015). Chemical or genetic inhibition of the protease leads to impaired oxidative phosphorylation and selectively kills AML cells and stem cells over normal hematopoietic cells in vitro and in vivo (Cole et al., 2015).

In bacteria, naturally occurring antibiotics, acyldepsipeptides (ADEPs), hyperactivate ClpP by binding the protease at its interface with ClpX and opening the pore of the ClpP protease complex (Brotz-Oesterhelt et al., 2005). When activated by ADEPs, ClpP can degrade full-length substrates without its regulatory subunit ClpX. Indeed, these ClpP activators are cytotoxic to a variety of microbial species including dormant bacteria that are responsible for resistant chronic infections (Brotz-Oesterhelt et al., 2005; Conlon et al., 2013). Thus, the activity of ClpP needs to be tightly regulated to maintain cellular homeostasis.

### II. ASPECTS OF THE PRESENT EMBODIMENTS

Here, it was found that ClpP hyperactivation induces lethality in leukemias and lymphomas, due to selective proteolysis in subsets of the mitochondrial proteome that are involved in mitochondrial respiration and oxidative phosphorylation. In contrast, normal hematopoietic cells display resistance to ClpP hyperactivation, likely reflecting their decreased reliance on oxidative phosphorylation and greater spare reserve capacity in their respiratory chain, compared to AML cells (Sriskanthadevan et al., 2015).

ClpP interacting proteins were recently identified (Cole et al., 2015), but a comprehensive assessment of ClpP substrates had not been performed. Provided herein is a comprehensive list of interacting partners of mitochondrial ClpP in living cells obtained using chemical and genetic activation of ClpP in the BioD assay (Table 1). A subset of mitochondrial enzymes, including subunits of the respiratory chain complexes, are selectively sensitive to ClpP-mediated degradation. Top hits in the BioTD assays were complex I subunits.

TABLE 1
Effect of ClpP activation after treatment with 0.6 μM ONC201 or
expression of Y118A ClpP mutant on degradation of mitochondrial
peptides in HEK293TREX cells detected by BioID mass spectrometry.

| | ClpP + Drug | | ClpP − Y118A | |
|---|---|---|---|---|
| | Log2 Fold change | p- | Log2 Fold change | p- |
| Gene Symbol | (vs control) | value | (vs control) | value |
| VWA8 | 1.69 | 0.00 | 1.02 | 0.00 |
| NFS1 | 0.55 | 0.00 | 0.25 | 0.03 |
| HSPA1L | 0.50 | 0.00 | −0.71 | 0.31 |
| PNPT1 | 0.32 | 0.00 | −0.07 | 0.51 |
| MGME1 | 0.27 | 0.00 | 0.44 | 0.01 |
| SSBP1 | 0.07 | 0.46 | 0.89 | 0.00 |
| RNMTL1 | 0.05 | 0.78 | −0.20 | 0.44 |
| HSD17B10 | 0.01 | 0.84 | 0.72 | 0.00 |
| LYRM4 | 0.00 | 1.00 | 0.52 | 0.00 |
| MTPAP | −0.10 | 0.59 | −0.78 | 0.11 |
| PIN1 | −0.11 | 0.65 | −3.81 | 0.00 |
| SUPV3L1 | −0.12 | 0.44 | 0.56 | 0.13 |
| AFG3L2 | −0.14 | 0.23 | 0.43 | 0.04 |
| ARG2 | −0.16 | 0.49 | 0.78 | 0.01 |
| ALDH2 | −0.17 | 0.04 | −0.18 | 0.33 |
| BCS1L | −0.17 | 0.08 | 0.47 | 0.00 |
| GCDH | −0.17 | 0.19 | 1.00 | 0.00 |
| CARS2 | −0.21 | 0.06 | −0.38 | 0.09 |
| PYCR2 | −0.21 | 0.03 | 0.26 | 0.04 |
| MDH2 | −0.24 | 0.00 | −0.03 | 0.51 |
| SLIRP | −0.25 | 0.09 | 0.25 | 0.17 |
| NIPSNAP2 | −0.31 | 0.20 | 0.61 | 0.03 |
| HSPE1 | −0.36 | 0.01 | 0.92 | 0.00 |
| MMAB | −0.37 | 0.00 | 0.11 | 0.04 |
| MTHFD1L | −0.39 | 0.05 | 0.25 | 0.12 |
| ABCB7 | −0.45 | 0.05 | −0.65 | 0.00 |
| PTPMT1 | −0.48 | 0.02 | −0.04 | 0.81 |
| CLIC4 | −0.49 | 0.27 | −2.81 | 0.01 |
| SHMT2 | −0.51 | 0.00 | −0.25 | 0.06 |
| T1MM44 | −0.52 | 0.00 | −0.12 | 0.26 |
| FASTKD2 | −0.62 | 0.00 | 0.71 | 0.00 |
| NDUFAF5 | −0.68 | 0.00 | −0.47 | 0.00 |
| GLS | −0.71 | 0.00 | 0.21 | 0.02 |
| SUCLA2 | −0.71 | 0.02 | −0.06 | 0.78 |
| K1AA0564 | −0.71 | 0.00 | 0.29 | 0.19 |
| HSDL2 | −0.71 | 0.00 | 0.83 | 0.00 |
| NDUFAF3 | −0.73 | 0.01 | 0.46 | 0.09 |
| ACAD9 | −0.76 | 0.02 | 1.18 | 0.01 |
| SDHB | −0.82 | 0.001 | −0.60 | 0.00 |
| THEM4 | −0.88 | 0.01 | −0.17 | 0.38 |
| ACAA2 | −0.88 | 0.00 | −0.92 | 0.00 |
| LETM1 | −0.94 | 0.00 | 1.06 | 0.00 |
| SDHA | −0.94 | 0.001 | 0.34 | 0.03 |
| NUDT1 | −1.00 | 0.06 | −3.17 | 0.00 |
| IBA57 | −1.05 | 0.00 | 1.65 | 0.00 |
| AK3 | −1.05 | 0.00 | −0.28 | 0.02 |
| ACADM | −1.06 | 0.00 | −0.40 | 0.01 |
| GFM1 | −1.08 | 0.00 | −0.26 | 0.06 |
| NDUFA6 | −1.08 | 0.00 | 0.44 | 0.00 |
| NDUFAF4 | −1.11 | 0.01 | 1.06 | 0.00 |
| NME4 | −1.16 | 0.00 | −0.42 | 0.05 |
| HINT2 | −1.17 | 0.00 | 0.09 | 0.67 |
| C7orf55 | −1.18 | 0.07 | −0.74 | 0.10 |
| C8orf82 | −1.19 | 0.02 | 1.60 | 0.00 |
| NIPSNAP1 | −1.19 | 0.05 | 0.11 | 0.77 |
| C20orf7 | −1.21 | 0.02 | −0.06 | 0.84 |
| PMPCA | −1.21 | 0.00 | 0.15 | 0.60 |
| MRPS28 | −1.24 | 0.00 | −0.49 | 0.09 |
| SPRYD4 | −1.26 | 0.00 | 0.22 | 0.02 |
| WARS2 | −1.27 | 0.00 | 0.25 | 0.26 |
| EARS2 | −1.29 | 0.01 | −0.17 | 0.70 |
| HADH | −1.30 | 0.00 | 0.80 | 0.03 |
| HARS2 | −1.30 | 0.00 | −0.49 | 0.00 |
| RG9MTD1 | −1.30 | 0.00 | 0.47 | 0.00 |
| ETFB | −1.35 | 0.00 | 0.40 | 0.05 |
| NARS2 | −1.36 | 0.00 | −0.14 | 0.32 |
| PPA2 | −1.38 | 0.00 | 0.02 | 0.87 |
| LYRM7 | −1.38 | 0.00 | 0.23 | 0.11 |
| MTHFD2 | −1.43 | 0.00 | 0.22 | 0.10 |
| SUCLG1 | −1.44 | 0.00 | −0.28 | 0.08 |
| ECHS1 | −1.49 | 0.00 | −0.33 | 0.06 |
| RTN4IP1 | −1.50 | 0.00 | −0.52 | 0.00 |
| ABHD10 | −1.53 | 0.00 | −0.16 | 0.27 |
| THNSL1 | −1.55 | 0.00 | 0.03 | 0.88 |
| HIBCH | −1.55 | 0.02 | 0.35 | 0.23 |
| FECH | −1.56 | 0.00 | −0.68 | 0.00 |
| TFAM | −1.61 | 0.00 | 0.63 | 0.10 |
| MRM3 | −1.62 | 0.00 | 0.14 | 0.24 |
| CLYBL | −1.63 | 0.00 | −3.17 | 0.00 |
| GLUD1 | −1.63 | 0.00 | −0.39 | 0.00 |
| XPNPEP3 | −1.63 | 0.00 | −0.31 | 0.60 |
| ACADSB | −1.76 | 0.00 | −0.20 | 0.44 |
| NDUFS7 | −1.81 | 0.00 | −0.08 | 0.41 |
| PPIF | −1.81 | 0.00 | 0.09 | 0.22 |
| GATB | −1.84 | 0.00 | −3.10 | 0.00 |
| NDUFAF7 | −1.89 | 0.00 | 0.04 | 0.76 |
| ADCK3 | −1.91 | 0.00 | −0.30 | 0.23 |
| IDE | −1.97 | 0.00 | −0.15 | 0.44 |
| IDH3A | −2.00 | 0.01 | −0.06 | 0.92 |
| NADKD1 | −2.04 | 0.00 | −1.04 | 0.01 |
| PYCR1 | −2.16 | 0.00 | 1.08 | 0.00 |
| ATPAF1 | −2.17 | 0.00 | 0.59 | 0.01 |
| ALDH4A1 | −2.21 | 0.00 | −0.81 | 0.00 |
| IARS2 | −2.38 | 0.00 | 0.43 | 0.00 |
| C2orf56 | −2.44 | 0.00 | 0.19 | 0.09 |
| NDUFS2 | −2.44 | 0.00 | 0.10 | 0.63 |
| OXCT1 | −2.58 | 0.00 | −0.95 | 0.00 |
| ATPAF2 | −2.58 | 0.00 | −0.91 | 0.06 |
| GTPBP3 | −2.58 | 0.00 | −0.50 | 0.29 |
| MRPS36 | −2.58 | 0.00 | 0.11 | 0.07 |
| NDUFS8 | −2.73 | 0.00 | 0.19 | 0.18 |
| GBAS | −2.79 | 0.01 | −0.38 | 0.41 |
| COX5A | −2.87 | 0.00 | 0.45 | 0.04 |
| QRSL1 | −2.90 | 0.00 | −0.01 | 0.90 |
| POLRMT | −2.95 | 0.00 | 0.57 | 0.05 |
| OXA1L | −3.00 | 0.00 | −1.00 | 0.02 |
| PET112 | −3.03 | 0.00 | −0.30 | 0.03 |
| SUCLG2 | −3.08 | 0.00 | −0.49 | 0.01 |
| NUDT19 | −3.10 | 0.00 | −0.58 | 0.01 |
| MRPL12 | −3.32 | 0.00 | 0.21 | 0.03 |
| ZADH2 | −3.46 | 0.00 | −1.46 | 0.01 |
| C20orf72 | −3.52 | 0.00 | 0.12 | 0.42 |
| OGDH | −3.70 | 0.00 | 1.67 | 0.00 |
| C12orf10 | −3.75 | 0.00 | −3.75 | 0.00 |
| DCAKD | −3.75 | 0.00 | −3.75 | 0.00 |
| NDUFA7 | −3.81 | 0.00 | −2.22 | 0.01 |
| SDHAF3 | −3.82 | 0.00 | −0.03 | 0.81 |
| CRAT | −4.00 | 0.00 | −3.00 | 0.00 |
| MRPL54 | −4.00 | 0.00 | −3.00 | 0.00 |
| GUF1 | −4.04 | 0.00 | 0.38 | 0.14 |
| ACAD10 | −4.09 | 0.00 | −4.09 | 0.00 |
| FOXRED1 | −4.09 | 0.00 | −4.09 | 0.00 |
| NUBPL | −4.17 | 0.00 | −2.58 | 0.01 |
| CDK5RAP1 | −4.17 | 0.00 | −0.26 | 0.32 |
| MRPL48 | −4.17 | 0.00 | 0.15 | 0.79 |
| NADK2 | −4.24 | 0.00 | 0.06 | 0.77 |
| MTRF1 | −4.25 | 0.00 | −1.44 | 0.03 |
| MRPS17 | −4.25 | 0.00 | −0.55 | 0.28 |
| MRPS11 | −4.25 | 0.00 | −0.34 | 0.44 |
| PITRM1 | −4.25 | 0.00 | −0.16 | 0.59 |
| MICU2 | −4.31 | 0.00 | −0.18 | 0.07 |
| NDUFAF1 | −4.32 | 0.00 | −4.32 | 0.00 |
| NFUl | −4.32 | 0.00 | −0.32 | 0.32 |
| POLG | −4.32 | 0.00 | 0.26 | 0.54 |
| BCKDHA | −4.32 | 0.00 | 1.68 | 0.00 |
| ACSS1 | −4.46 | 0.00 | −1.46 | 0.08 |
| MPST | −4.46 | 0.01 | −1.29 | 0.08 |
| MRPL10 | −4.46 | 0.00 | −0.21 | 0.54 |
| MRPL21 | −4.52 | 0.00 | −0.72 | 0.34 |
| MRPS6 | −4.52 | 0.00 | −0.62 | 0.15 |
| MARS2 | −4.52 | 0.00 | 0.23 | 0.55 |
| UQCRB | −4.70 | 0.00 | −2.38 | 0.00 |
| COQ8A | −4.74 | 0.00 | −0.67 | 0.00 |
| MRPS24 | −4.81 | 0.00 | 1.12 | 0.00 |
| TAC01 | −4.82 | 0.00 | 0.07 | 0.63 |
| MRPS15 | −4.91 | 0.00 | −2.32 | 0.01 |
| GTPBP10 | −4.91 | 0.00 | −0.91 | 0.10 |
| BCKDHB | −4.91 | 0.00 | 0.79 | 0.01 |
| PDPR | −4.95 | 0.00 | 0.09 | 0.69 |
| COX5B | −5.00 | 0.00 | 0.46 | 0.03 |
| MRPL44 | −5.04 | 0.00 | 1.13 | 0.00 |
| MRPL40 | −5.09 | 0.00 | 0.37 | 0.04 |
| GLRX5 | −5.21 | 0.00 | −0.26 | 0.24 |
| MRPL55 | −5.25 | 0.00 | −0.20 | 0.37 |
| DHTKD1 | −5.25 | 0.00 | −0.12 | 0.56 |
| POLG2 | −5.29 | 0.00 | −0.89 | 0.01 |
| PCK2 | −5.32 | 0.00 | −0.51 | 0.05 |
| ECSIT | −5.36 | 0.00 | −1.04 | 0.04 |
| ACN9 | −5.46 | 0.00 | −2.14 | 0.00 |
| ACO2 | −5.46 | 0.00 | −0.29 | 0.28 |
| MRRF | −5.49 | 0.00 | −0.10 | 0.44 |
| GRPEL1 | −5.52 | 0.00 | 0.00 | 1.00 |
| VARS2 | −5.52 | 0.00 | 0.76 | 0.01 |
| TST | −5.55 | 0.00 | −0.51 | 0.24 |
| MTIF2 | −5.55 | 0.00 | 0.30 | 0.31 |
| AARS2 | −5.73 | 0.00 | −0.14 | 0.58 |
| PDE12 | −5.78 | 0.00 | 0.03 | 0.94 |
| PAM16 | −5.91 | 0.00 | −0.42 | 0.39 |
| NDUFS4 | −5.93 | 0.00 | −0.57 | 0.01 |
| NDUFV2 | −5.96 | 0.00 | −0.47 | 0.00 |
| GATC | −5.98 | 0.00 | −0.05 | 0.76 |
| ALDH1L2 | −6.04 | 0.00 | −0.69 | 0.00 |
| MRPL46 | −6.09 | 0.00 | −0.28 | 0.26 |
| ERAL1 | −6.19 | 0.00 | −1.33 | 0.00 |
| NDUFS6 | −6.32 | 0.00 | −0.26 | 0.08 |
| NDUFA2 | −6.32 | 0.00 | −0.23 | 0.06 |
| GRSF1 | −6.34 | 0.00 | 1.91 | 0.00 |
| MRPS26 | −6.38 | 0.00 | −0.52 | 0.05 |
| FMC1 | −6.86 | 0.00 | 0.41 | 0.13 |
| NDUFA12 | −7.55 | 0.00 | −1.02 | 0.00 |
| NDUFAF2 | −7.94 | 0.00 | −0.15 | 0.01 |
| NDUFV3 | −8.18 | 0.00 | 0.05 | 0.45 |
| RPS15A | −0.69 | 0.01 | −0.45 | 0.03 |
| SLC27A2 | −1.81 | 0.05 | −3.81 | 0.00 |
| MRPS7 | −2.77 | 0.00 | 0.71 | 0.00 |
| MRPL14 | −3.46 | 0.00 | 0.00 | 1.00 |
| METTL17 | −3.58 | 0.00 | −3.58 | 0.00 |
| ALDH6A1 | −3.81 | 0.00 | −1.22 | 0.09 |
| MRPS16 | −3.81 | 0.00 | 0.10 | 0.92 |
| MTRF1L | −3.81 | 0.00 | 0.51 | 0.25 |
| MRPS25 | −3.91 | 0.00 | 0.62 | 0.09 |
| NMNAT3 | −4.00 | 0.00 | −4.00 | 0.00 |
| MRPL45 | −6.44 | 0.00 | −0.29 | 0.03 |
| LARS2 | −0.14 | 0.47 | 0.10 | 0.73 |
| IDI1 | −0.22 | 0.54 | 1.28 | 0.01 |
| CPOX | −2.12 | 0.04 | 0.88 | 0.07 |
| ABAT | −2.58 | 0.01 | −3.58 | 0.00 |
| CHCHD3 | −3.46 | 0.00 | −2.46 | 0.01 |
| PRKCA | −3.58 | 0.00 | −3.58 | 0.00 |
| GPT2 | −3.70 | 0.00 | 0.21 | 0.55 |
| COQ6 | −3.70 | 0.00 | 0.62 | 0.11 |
| PDIA3 | 0.03 | 0.76 | −0.41 | 0.00 |
| HSD17B8 | −1.26 | 0.14 | −2.58 | 0.03 |
| DHRS4 | −2.32 | 0.03 | 0.14 | 0.79 |
| TARS2 | −1.95 | 0.00 | −0.25 | 0.38 |
| SFXN4 | −0.58 | 0.07 | −0.38 | 0.04 |
| AIFM1 | −1.58 | 0.00 | −2.32 | 0.00 |
| ACADVL | 0.55 | 0.01 | 0.28 | 0.12 |
| MRPS23 | −1.58 | 0.00 | −0.22 | 0.23 |
| MRPL19 | −3.51 | 0.00 | −0.97 | 0.01 |
| TBRG4 | 0.28 | 0.06 | 0.28 | 0.47 |
| ACAT1 | −0.59 | 0.00 | −0.01 | 0.89 |
| PDK3 | −1.55 | 0.01 | 0.60 | 0.08 |
| COX4I1 | −2.74 | 0.00 | 0.17 | 0.27 |
| MRPL47 | −4.52 | 0.00 | −1.94 | 0.01 |
| ABCB10 | −0.24 | 0.41 | −1.32 | 0.01 |
| EFHA1 | −0.47 | 0.64 | −4.17 | 0.03 |
| FASTKD5 | −4.91 | 0.00 | 1.05 | 0.01 |

Indeed, ClpP activation functionally inhibited complex I most effectively, compared to complex II and IV, which were also inhibited but to a lesser degree. As a result, ClpP activation damaged mitochondria morphologically and functionally through structural disruption of cristae, inhibition of oxidative phosphorylation, and accumulation of mitochondrial ROS, resulting in anti-tumor effects. Considering several recent reports showing that cancer stem and chemo-resistant cells rely highly on oxidative phosphorylation (Farge et al., 2017; Kuntz et al., 2017; Lagadinou et al., 2013; Marin-Valencia et al., 2012; Viale et al., 2014), it was speculated that this therapeutic approach may also have potential to eliminate chemo-resistant populations of malignant cells and prevent relapse of the disease.

Deletions or mutations of ClpP have never been reported in primary AML, suggesting that ClpP could be an effective target across the spectrum of molecular and cytogenetic subsets of AML. However, patient samples with the lowest levels of ClpP are less sensitive to ClpP hyperactivation. Thus, levels of ClpP serve as a biomarker to select patients most and least likely to respond to this therapy.

Genetic systems were established to activate and inactivate human ClpP by identifying certain point mutations. The Y118A mutation in human ClpP leads to constitutive hyperactivation of the protease. The D190A mutation is present in ONC201-resistant cells and is an inactivating mutation both in vitro and in cellular assays.

The present drug screen identified agonists of mitochondrial ClpP that are more potent than the antibiotic agents ADEPs. The most potent activator imipridones (e.g., ONC201 and ONC212) are a novel class of anti-cancer compounds, which effectively kill cancer cells but are much less toxic to normal cells (Allen et al., 2013; Ishizawa et al., 2016). Their efficacy is independent of TP53 mutation status (Allen et al., 2013; Ishizawa et al., 2016). While the preclinical efficacy of these compounds has been established in numerous cancers, the direct target was elusive. The dopamine receptor DRD2 has been suggested as a putative target of ONC201 (Kline et al., 2016; Kline et al., 2018), based on homology modeling and a cellular β-arrestin assay but not based on evidence of direct binding. Also, DRD2 knock-out cells can be sensitive to ONC201 (Kline et al., 2018), suggesting that it may not be the functionally critical mechanism of action. The crystal structure of the ClpP-ONC201 complex confirmed ClpP as a direct target for ONC201 and identified its binding pocket. It also showed that ONC201-mediated activation of ClpP has global structural effects that go beyond those of ADEP-mediated activation (Gersch et al., 2015; Lee et al., 2010). Drug binding not only widened the axial entrance pore but also opened up channel-like pores on the “side wall” of the assembled protease. The mechanism of peptide products' escape from the ClpP reaction chamber has been debated in the literature (Sprangers et al., 2005). The new opening, together with the increased dynamics of this region, suggests that these pores could provide a convenient escape route for cleaved peptide products and could help the ClpP machinery to prevent peptide accumulation in the degradation chamber. ONC201-binding not only increases the dynamics of the ClpP N-terminal residues, a region well known as a major regulatory site crucial for ClpX-mediated activation (Kang et al., 2004), but also induces major conformational changes at the heptamer-heptamer interface with direct effects on the active site region. ADEPs structural effects of activation of bacterial ClpP are most pronounced in the apical region of the protein with the heptamer-heptamer interface largely undisturbed (Gersch et al., 2015; Lee et al., 2010). Collectively, these findings provide an explanation for the recent report demonstrating that ONC201 reduces oxidative phosphorylation (Greer et al., 2018). A refined model of the ClpP-ONC212 complex suggests that its trifluoromethyl substituent enhances ONC212's potency through increased binding affinity and improved structural complementarity to ClpP.

Genetic ClpP activation results in ATF4 increase in Z138 cells without an increase of phosphorylated eIF2α, validating the recent finding that ONC201 induces atypical integrated stress response (ISR), characterized by eIF2α-independent induction of ATF4 unlike eIF2α-dependent classical ISRs (Ishizawa et al., 2016). This is also consistent with another recent report of eIF2α-independent ATF4 induction being downstream of mitochondrial unfolded stress response (UPRmt) (Munch and Harper, 2016), and reflecting that activation of human ClpP phenocopies UPRmt. Unlike ClpP functionality reported in C. elegans (Haynes et al., 2007; Quiros et al., 2016), several reports suggest that ClpP may not be a master regulator of signaling.

ONC201 is currently in early phase clinical trials against AML and other cancers (Arrillaga-Romany et al., 2017; Kline et al., 2016; Stein et al., 2017), to determine safety and optimal dosing schedule. An early example of blast reduction in a patient with AML is shown in FIG. S10, but these trials are still ongoing. In some solid tumors, in particular in gliomas, the trials have demonstrated promising clinical responses without serious adverse events. Thus, the present findings related to ONC201 as a ClpP activator can immediately be validated in ongoing clinical trials in patients, and potentially also be tested in future clinical trials of its improved analogues, which include ONC206 (Wagner et al., 2017) and ONC212. Of note, ample evidence suggests lethality in TP53 wild-type and mutant tumors (Allen et al., 2013; Ishizawa et al., 2016; Kline et al., 2016). Moreover, in conjunction with previous reports showing that ADEPs are promising antibiotics and that ADEP-resistant strains of staphylococcal isolates are rare (Conlon et al., 2013), imipridones may exert effective antibiotic properties.

Of note, neither Perrault Syndrome patients, who carry inactivating mutations of ClpP, nor ClpP-deficient mice develop tumors. Inhibiting ClpP in leukemic cells leads to the accumulation of misfolded or damaged respiratory chain complex subunits that impair respiratory chain activity and causes cell death. In contrast, hyperactivating ClpP in cancer cells increases degradation of respiratory chain complex subunits leading to impaired respiratory chain activity. Thus, ClpP needs to be tightly regulated in malignancies as both inhibition and hyperactivation of ClpP impairs respiratory chain activity and causes cell death, although through different mechanisms.

In conclusion, hyperactivation of ClpP is a novel therapeutic strategy against hematologic and solid tumors, which induces selective proteolysis of particular subsets of mitochondrial matrix proteins, resulting in prominent anti-tumor effects. Currently the most potent ClpP activators, imipridones, are being evaluated in clinical trials.

### III. METHODS OF TREATMENT

A. Cancer

The present invention provides methods of treating a cancer patient with an agent that activates mitochondrial proteolysis, such as an imipridone (e.g., ONC201, ONC212, or ONC206; see, for example, U.S. Pat. Nos. 9,845,324 and 10,172,862, each of which is incorporated herein by reference in its entirety). Such treatment may also be in combination with another therapeutic regime, such as chemotherapy or immunotherapy. Certain aspects of the present invention can be used to select a cancer patient for treatment based on the level of ClpP expression in the patient's tumor and/or the presence of inactivating mutations (e.g., D190A) in ClpP in the patient's tumor. In various aspects, about 5%, 10%, 15%, 20%, 25%, 30%, 35%, 40%, 45%, 50%, 55%, 60%, 65%, 70%, 75%, 80%, 85%, 90%, 95%, or 100% of the cells that comprise the cancer may harbor a ClpP expression level or mutation status that indicates that the patient is a candidate for treatment. In other aspects, various percentages of cells comprising the cancer may harbor a marker that indicates that the patient is a candidate for treatment. Other aspects of the present invention provide for selecting a cancer patient for treatment based on the patient having previously failed to respond to the administration of an anti-cancer therapy.

The term “subject” or “patient” as used herein refers to any individual to which the subject methods are performed. Generally the patient is human, although as will be appreciated by those in the art, the patient may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The methods described herein are useful in treating cancer. Generally, the terms “cancer” and “cancerous” refer to or describe the physiological condition in mammals that is typically characterized by unregulated cell growth. More specifically, cancers that are treated in connection with the methods provided herein include, but are not limited to, solid tumors, metastatic cancers, or non-metastatic cancers. In certain embodiments, the cancer may originate in the lung, kidney, bladder, blood, bone, bone marrow, brain, breast, colon, esophagus, duodenum, small intestine, large intestine, colon, rectum, anus, gum, head, liver, nasopharynx, neck, ovary, pancreas, prostate, skin, stomach, testis, tongue, or uterus.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, previous therapy, the patient's clinical history and response to the agent, and the discretion of the physician. The agent may be suitably administered to the patient at one time or over a series of treatments.

1. Combination Treatments

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an imipridone is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

a. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); venetoclax (ABT-199); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DFMO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

c. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311). It is contemplated that one or more anti-cancer therapies may be employed with the antibody therapies described herein.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), programmed death-ligand 1 (PD-L1), T-cell immunoglobulin domain and mucin domain 3 (TIM-3), and V-domain Ig suppressor of T cell activation (VISTA). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO©, is an anti-PD-1 antibody described in WO2006/121168. Pembrolizumab, also known as MK-3475, Merck 3475, lambrolizumab, KEYTRUDA©, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

d. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

e. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

B. Bacterial Infection

In one embodiment, methods of killing bacteria are provided. The methods comprise the step of applying a safe and effective amount an imipridone to a bacterial cell. Determining the minimum inhibitory concentration (MIC) is well known in the art. Antibacterial efficacy of drugs is typically measured by determining in vitro the MIC of the drug for the individual bacterial species of interest. Thus, a therapeutically effective amount of an imipridone includes an amount that is above the MIC for the infection being treated. If more than one pathogen is present, the effective amount of an imipridone would be greater than or equal to the highest MIC of the infecting organisms. Generally, therapeutic regimens for bacterial infections are predicated upon administering one or more drug doses to the patient that achieve drug concentrations (in, for example, the blood) that at least meet and preferably exceed the MIC for at least a portion of the dosing interval. In some cases, the dosage may be maintained at the same level throughout the course of therapy or adjusted to increase or decrease the amount administered. In some aspects, the imipridone dosage is not increased due to developing resistance (but may be increased for purposes of administering the appropriate dose during therapy).

As is common with pharmaceutical agents, the prophylactic or therapeutic dose of the antibacterial drug used in the treatment of a bacterial infection will vary with the severity of the infection and the route by which the drug is administered. The dose, and perhaps the dose frequency, will also vary according to the age, body weight, and response of the individual patient. The optimal dosage of an imipridone can be readily determined by those of skill in the art, and can be defined in a variety of ways.

Bacteria against which the method of the present application can be used include both gram-positive and gram-negative genera. Gram-positive genera against which the method can be used include Staphylococcus, Streptococcus, Enterococcus, Clostridium, Haemophilus, Listeria, Corynebacterium, Bifidobacterium, Eubacterium, Lactobacillus, Leuconostoc, Pediococcus, Peptostreptococcus, Propionibacterium, and Actinomyces.

Particular gram-positive species against which the method can be used include S. aureus (including methicillin-resistant S. aureus), S. epidermidis, S. haemolyticus, S. hominis, S. saprophyticus, S. pneumoniae, S. pyogenes, S. agalactiae, S. avium, S. bovis, S. lactis, S. sangius, E. faecalis, E. faecium, C. difficile, C. clostridiiforme, C. innocuum, C. perfringens, C. ramosum, L. monocytogenes, C. jeikeium, E. aerofaciens, E. lentum, L. acidophilus, L. casei, L. plantarum, P. anaerobius, P. asaccarolyticus, P. magnus, P. micros, P. prevotil, P. productus, and P. acnes.

Clinically the salient pathogens include positive species against which the method can be used include S. aureus (including methicillin-resistant S. aureus), S. epidermidis, S. haemolyticus, S. pneumoniae, S. pyogenes, S. agalactiae, E. faecalis, E. faecium, C. difficile, C. clostridiiforme, C. perfringens, and L. monocytogenes.

C. Perrault Syndrome

Perrault syndrome is a sex-influenced disorder characterized by bilateral sensorineural hearing loss (SNHL) in both males and females and ovarian dysgenesis in females. Fertility in affected males is reported as normal. Some patients also have neurologic manifestations, including learning difficulties and developmental delay, cerebellar ataxia, and motor and sensory peripheral neuropathy. Type I Perrault syndrome is static and without neurologic disease. Type II Perrault syndrome is progressive with neurologic disease.

SNHL is bilateral, is caused by changes in the inner ear, and ranges from profound with prelingual (congenital) onset to moderate with early-childhood onset. When onset is in early childhood, hearing loss can be progressive.

Females with Perrault syndrome have abnormal or missing ovaries (ovarian dysgenesis), although their external genitalia are normal. Severely affected girls do not begin menstruation by age 16 (primary amenorrhea), and most never have a menstrual period. Less severely affected women have an early loss of ovarian function (primary ovarian insufficiency); their menstrual periods begin in adolescence, but they become less frequent and eventually stop before age 40. Women with Perrault syndrome may have difficulty conceiving or be unable to have biological children.

Perrault syndrome has several genetic causes. TWNK, CLPP, HARS2, LARS2, or HSD17B4 gene mutations have been found in a small number of affected individuals. This condition is inherited in an autosomal recessive pattern, which means both copies of the gene in each cell have mutations.

Inactivating mutations in both CLPP and HSDI7B4 were identified in imipridone-resistant cells. Inactivating point mutations in CLPP that cause interruption of ClpX binding have been reported in patients with Perrault syndrome. As such, since imipridones can activate ClpP without ClpX, activation of ClpP by imipridones may recover the inactivated CLPP function in patients with Perrault syndrome, and thus, be therapeutically beneficial for such patients. In addition, activation of ClpP by imipridones may bypass inactivating HSD17B4 mutations in patients with Perrault syndrome. Thus, ClpP activation by imipridones may improve the symptoms, or prevent the occurrence/aggravation of symptoms, such as hearing loss and ovarian dysfunction/infertility, in patients with Perrault syndrome, and in particular in patients with Perrault syndrome caused by mutations in CLPP or HSDI7B4.

### IV. KITS

In various aspects of the invention, a kit is envisioned containing, diagnostic agents, therapeutic agents and/or delivery agents. In some embodiments, the kit may comprise reagents for assessing a patient selection marker, such as a ClpP expression level or mutation status, in a patient sample. In some embodiments, the present invention contemplates a kit for preparing and/or administering a therapy of the invention. The kit may comprise reagents capable of use in administering an active or effective agent(s) of the invention. Reagents of the kit may include one or more anti-cancer component of a combination therapy, as well as reagents to prepare, formulate, and/or administer the components of the invention or perform one or more steps of the inventive methods.

In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass. The kit may further include an instruction sheet that outlines the procedural steps of the methods, and will follow substantially the same procedures as described herein or are known to those of ordinary skill.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials & Methods

Mice. For all the animal studies in the present study, the study protocols were approved by the Institutional Animal Care and Use Committee (IACUC) at the Princess Margaret Cancer Centre and MD Anderson Cancer Center. Two million Z138 cells transfected with the wild-type or D190A mutant CLPP-over-expressing vector and labeled with luciferase were injected to individual NSG mice (n=7 per treatment group, all female). After confirming engraftment measured by in vivo bioluminescence imaging on d 9 post-transplantation, ONC212 (50 mg/kg/d) or vehicle (water) is administered by oral gavage every other d until the mice got moribund. Tumor burden measured by luminescence was followed weekly until day 31. Independently, two million Z138 cells transfected with tetracycline-inducible Y118A mutant ClpP were labeled with luciferase and injected to individual NSG mice (n=10 per treatment group, all male). After confirming engraftment measured by in vivo bioluminescence imaging on day 5 post-transplantation, the mice were treated with or without tetracycline (2 mg/mL) in drinking water until moribund. One million OCI-AML2 cells were injected to individual SCID mice (n=10 per treatment group, all male). Five days after injection, mice were treated with ONC201 twice daily with ONC201 by oral gavage (100 mg/kg) for 13 days. Engraftment experiments using patient-derived xenograft AML cells were performed as previously reported (Ishizawa et al., 2016). Primary AML cells were transplanted into female 6-week old NSG mice, and leukemia cells were harvested from secondarily transplanted mice. Leukemic cells were treated with or without 250 nM of ONC212 for 36 hours, then 0.7 million trypan blue-negative cells were injected via tail vein into each of 7 NSG mice per treatment group. The mice in each group were monitored for survival.

Bacterial Cell Culture.

For the expression and purification of human mitochondrial ClpP protein E. coli SG1146 carrying pETSUMO2-CLPP(-MTS) were grown aerobically in Luria-Bertrani Broth (LB; 10 g/L tryptone, 5 g/L yeast extract, 10 g/L NaCl) supplemented with 50 μg/mL kanamycin at 37° C. with shaking at 180 rpm.

Protein Purification and Crystallization.

Human ClpP was expressed and purified as described previously (Kang et al., 2004) (Kimber et al., 2010; Wong et al., 2018). Wild type and mutant (Y118A and D190A) human ClpP (without mitochondrial targeting sequences) were cloned into pETSUMO2 expression vectors and expressed in E. coli SG1146 (Kimber et al., 2010). To induce protein expression, bacteria, after reaching OD600˜0.6, were treated with 1 mM isopropyl-1-thio-B-D-galactopyranoside (IPTG) for 4 h at 37° C., harvested by centrifugation, and disrupted in lysis buffer (25 mM Tris-HCl (pH 7.5), 0.5 M NaCl, 10 mM imidazole, 10% glycerol) by Emulsiflex C5 (4 passes; Avestin, Ottawa, Canada). Following cell lysis, the insoluble material was removed by centrifugation (26,892×g (Sorvall rotor SS-34) for 30 min) and the supernatant was passed through a 5 mL Ni sepharose high-performance (GE) column pre-equilibrated with lysis buffer. The protein was eluted with 40 mM imidazole, diluted with 2 mL of dialysis buffer (25 mM Tris-HCl (pH 7.5), 0.1 M NaCl, 10% glycerol), mixed with SUMO protease (1:100; Lee et al., 2008), and dialyzed overnight at 4° C. with light stirring into 4 L of dialysis buffer using SnakeSkin 10K dialysis membrane (ThermoScientific, Waltham, Mass.). The dialyzed material was then passed through a second 5 mL Ni-column (ThermoScientific, Waltham, Mass.) and the flow-through solution containing untagged ClpP was collected. All collected fractions were analyzed by SDS-PAGE.

For crystallography, protein was concentrated using Amicon Ultra-15 30K concentrator (Sigma-Aldrich), and further purified using an anion exchange 5 mL QSepharoseHP HiTrap (Amersham Biosciences, Little Chalfont, UK) column with a linear gradient from 100 mM to 1 M NaCl in 20 mM Tris-HCl (pH 7.5). Protein eluted at about 200 mM NaCl concentration. It was then pre-concentrated using Amicon Ultra-15 30K concentrators and dialyzed at 4° C. overnight into 25 mM Bis-Tris, pH 6.5, containing 3 mM DTT. ClpP was then further concentrated to a final concentration of 12 mg/mL. ONC201, solubilized in 100% DMSO, was added to the concentrated protein to bring the final concentration of the compound to 2.5 mM with a DMSO concentration of 5%.

The ClpP-ONC201 complex was crystallized at 4° C. by the hanging drop vapor diffusion method. 2 μL of protein-drug solution were mixed with 2 μL of reservoir solution. Wells containing reservoir solutions of 500 μL of 5% (w/v) PEG 4,000, 100 mM KCl, and 100 mM NaAc (pH 5.2) produced crystals of 100-200 μm in all three dimensions. Crystals appeared in 2-3 weeks and were harvested into reservoir solution containing 5% (w/v) PEG 4,000, 100 mM KCl, and 100 mM NaAc (pH 5.2), 2.5 mM ONC201, and 5% DMSO; 20% glycerol was added for cryo-protection. Crystals in standard cryo-loops were flash-frozen in liquid nitrogen.

Collection and Processing of Diffraction Data.

Diffraction data were obtained at beamline 08ID-1 of the Canadian Light Source (Saskatoon, Canada) at 100 K and recorded with the help of a Pilatus3 S 6M detector (Dectris, Switzerland). The wavelength was 0.97949 Å and 2500 images were collected with a 0.1° oscillation range and 0.2 s exposures. Crystal to detector distance was 392.6 mm. Data were indexed, integrated, and scaled using the XDS (Kabsch, 2010) and CCP4 (Winn et al., 2011) software packages. The protein complex crystallized in space group C2 with one ClpP heptamer ring in the asymmetric unit (ASU), as had previously been seen for the closed conformation of human mitochondrial ClpP (PDB-ID:1TG6) (Kang et al., 2004).

Structure Solution and Refinement.

The crystal structure of the ClpP-ONC201 complex was solved by molecular replacement using the PHENIX software package (Adams et al., 2010; McCoy et al., 2007). The same software was applied for refinement and validation and the package COOT (Emsley and Cowtan, 2004; Emsley et al., 2010) for model building. Starting phases for structure determination were calculated using the activated ClpP heptamer structure with waters removed as the search model (PDB: 6BBA; Wong et al., 2018). Riding hydrogens were used during the last several rounds of refinement (Afonine and Adams, 2012) to optimize the geometry but were not included in the final deposited coordinate file. See Table 2 for data reduction and refinement statistics. PyMol v1.3 software was used to generate structure figures (DeLano, 2002). Coordinates and structure factors of the CpP-NC201 complex structure have been deposited into the RCSB—Protein Data Bank with Accession No. 6DL7.

TABLE 2
Data collection and refinement statistics for ClpP ONC201 complex.
Statistics for the highest-resolution shell are shown in parentheses.

| Wavelength | 0.97949 Å |
|---|---|
| Resolution range (Å) | 49.4-2.0 (2.07-2.0) |
| Space group | C 1 2 1 |
| Unit cell (a, b, c (Å); α, β, γ (°)) | 142.4 153.4 104.8 90 117.6 90 |
| Total reflections | 635237 (63647) |
| Unique reflections | 133979 (13336) |
| Multiplicity | 4.7 (4.8) |
| Completeness (%) | 1.00 (1.00) |
| Mean I/sigma(I) | 5.64 (0.81) |
| Wilson B-factor | 37.1 |
| R-merge | 0.149 (1.58) |
| R-meas | 0.168 (1.78) |
| CC1/2 | 0.994 (0.504) |
| CC* | 0.998 (0.819) |
| Reflections used in refinement | 133638 (13076) |
| Reflections used for R-free | 6704 (668) |
| R-work | 0.2297 (0.3920) |
| R-free | 0.2622 (0.3902) |
| CC(work) | 0.948 (0.697) |
| CC(free) | 0.933 (0.704) |
| Number of non-hydrogen atoms | 10643 |
| macromolecules | 9687 |
| ligands | 203 |
| Protein residues | 1244 |
| R1VIS(bonds) | 0.01 |
| R1VIS(angles) | 0.57 |
| Ramachandran favored (%) | 96 |
| Ramachandran allowed (%) | 3.8 |
| Ramachandran outliers (%) | 0.25 |
| Rotamer outliers (%) | 2.3 |
| Clashscore | 1.56 |
| Average B-factor | 49.7 |
| macromolecules | 49.5 |
| ligands | 46.9 |
| solvent | 53 |
| Number of TLS groups | 1 |

Chemical Screen.

Assay buffer consisted of 25 mM HEPES, pH 7.4, 5 mM MgCl₂, 5 mM KCl, 0.03% Tween 20, 10% glycerol, 16 mM creatine phosphate, 13 U/ml creatine kinase, and 3 mM ATP. 1.0 μM human ClpP (Cole et al., 2015) was dissolved in the assay buffer using Biomek FX robotic liquid handler (Beckman Coulter Life Sciences, Indianapolis, Ind.) and mixed with 0.625 mM and 1.25 mM-concentrations of each compounds in 384-well plates using Beckman Multimek 96/384 liquid handling system (Beckman Coulter Life Sciences, Indianapolis, Ind.) at 0.2 μL per well (final concentrations 4.15 and 8.3 μM, respectively) and incubated at 37° C. for 10 min. Fluorescent tagged-substrate, FITC-casein (4.0 μM), was then added to each well and fluorescence was measured at 485/535 nm every 5 min for 70 min at 37° C. using PHERAstar microplate reader (BMG LABTECH, Ortenberg, Germany).

ClpP Enzymatic Assays.

Assay buffer consisted of 25 mM HEPES, pH 7.5, 5 mM MgCl₂, 5 mM KCl, 0.03% Tween 20, 10% glycerol, 16 mM creatine phosphate, 13 U/ml creatine kinase, and 3 mM ATP for FITC-Casein assay, 100 mM KCl, 5% glycerol, 10 mM MgCl₂, 20 mM Triton X-100, and 50 mM TRIS pH 8 for AC-WLA-AMC assay, 50 mM Tris, pH 8, 300 mM KCl, and 15% glycerol for Ac-Phe-hArg-Leu-ACC assay, 50 mM Hepes, pH 7.5 with 5 mM ATP, 0.03% Tween 20, 15 mM MgCl₂, 100 mM KCl and 5% Glycerol for FAPHMALVPV (Clptide) assay, and 25 mM Tris, pH 7.5 with 150 mM NaCl for MCA-Pro-Leu-Gly-Pro-D-Lys assay (Gersch et al., 2016).

For fluorescence assays, 0.7 μM (for FITC-casein, AC-WLA-AMC, and Ac-Phe-hArg-Leu-ACC assays) or 7.0 μM (for FAPHMALVPV and MCA-Pro-Leu-Gly-Pro-D-Lys assays) human ClpP was dissolved in the assay buffer, incubated at 37° C. for 10 min, and mixed with increasing concentrations of ONC201, ONC201 isomer, and ONC212 (0-100 μM) in 96 well plates at 50 μL per well in triplicate. Fluorescent tagged-substrates, FITC-casein (4.5 μM) or AC-WLA-AMC (15 mM) (Wong et al., 2018), Ac-Phe-hArg-Leu-ACC (100 μM), FAPHMALVPV (50 μM) and MCA-Pro-Leu-Gly-Pro-D-Lys (25 μM) were then added to each well and fluorescence was measured at 485/535 nm for FITC casein assay, at 360/440 nm for AC-WLA-AMC assay, at 380/440 nm for Ac-Phe-hArg-Leu-ACC assay, at 320/420 nm for FAPHMALVPV (Clptide) assay, and at 320/405 nm for MCA-Pro-Leu-Gly-Pro-D-Lys assay every 30 seconds for 90 min at 37° C. using a monochromator microplate reader (Clariostar BMG LABTECH, Ortenberg, Germany). Hill coefficient was determined using Origin7, Pharmacology—Dose-response curve with log (compound concentration) as the independent variable.

For gel-based assays, 1.5 μM ClpP, alone and in combination with 4.5 M ClpX, was mixed with 22 μM unlabeled bovine α-casein and treated with 0.2 and 6.3 μM concentrations of ONC201 and ONC212 in FITC-casein assay buffer. The mixture was incubated at 37° C. for 3 h, loaded on 12% SDS-PAGE, run at 120V, and stained with Coomassie Blue.

Isothermal Titration Calorimetry (ITC).

ITC binding measurements were performed using the MicroCal VP-ITC system (Malvern, Malvern, UK). Aliquots of purified wild type and D190A ClpP were dialyzed separately overnight at 4° C. with light stirring into 20 mM Tris-HCl, 5% DMSO, pH 7.65 (at room temperature) using SnakeSkin 10K dialysis membrane (ThermoFisher, Waltham, Mass.). The VP-ITC cell was filled with 20 μM ClpP (WT or D190A; ClpP monomer concentration) and 100 μM ONC201 was used in the syringe. The following setup was used: Injection volume: 281.55 μL, Cell volume: 1.4551 mL, Spacing time between injections: 240 s, 27 injections: 10 μL over 20 s each; 1st 2 μL over 4 s, filter period—2 s, steering speed—307, temperature—25° C., reference power—15 μCal/s. In the reversed experiment 500 μM WT ClpP in the syringe was titrated into 50 μM ONC201 solution; same instrument setup was used for these experiments. Control experiments were carried out to account for dilution effects upon ligand into protein and protein into ligand titration. Data were analyzed with Origin7 MicroCal Analysis software.

Gel Filtration.

0.4 mg of WT or D190A ClpP in 400 μL (with or without ONC201 in 1:1 molar ratio—ClpP monomer to ONC201 ratio; in running buffer) was loaded onto the analytical size exclusion column Superdex 200 10/300 GL (Amersham Biosciences, Little Chalfont, UK) and run at room temperature at 0.5 mL/min in the running buffer (20 mM TrisHCl, 100 mM NaCl, pH 7.5).

Cell Culture.

OCI-AML2 cells were grown in Iscove's Modified Dulbecco's Medium (IMDM) with 10% FBS. OCI-AML3, HCT116, OC316, and SUM159 cells were cultured in RPMI medium with 10% FBS. TEX cells (Warner et al., 2005) were provided by Dr. John Dick (Ontario Cancer Institute, Toronto, Canada) and grown in IMDM supplemented with 15% FCS, 2 mM L-glutamine, 20 ng/mL stem cell factor (SCF), and 2 ng/mL IL-3 (R&D Systems, Minneapolis, Minn.). Z138 cells were cultured in RPMI with 20% FBS. T-REx HEK293 cells were grown in DMEM with 10% FBS.

ClpP−/− and ClpP+/+ T-REx HEK293 cells were a gift from Dr. Aleksandra Trifunovic's lab (CECAD Research Center, University of Cologne, Germany). All the other cell lines were purchased from Leibniz-Institut Deutsche Sammlung von Mikroorganismen und Zellkulturen (DSMZ, Braunschweig, Germany) or the American Type Culture Collection (ATCC, Manassas, Va.).100 units/mL penicillin and 100 μg/mL streptomycin were added to the media for all cell lines. All cells were cultured at 37° C. and 5% CO₂. The authenticity of the cell lines was confirmed by DNA fingerprinting with the short tandem repeat method, using a PowerPlex 16 HS System (Promega, Madison, Wis.) within 6 m before the experiments.

Primary Cells.

Bulk AML cells from AML patients and peripheral blood stem cells from healthy G-CSF-treated stem cell donors were isolated by Ficoll density centrifugation and apheresis, respectively. Isolated cells were maintained in IMDM supplemented with 10% FBS, or Myelocult H5100 (Stemcell Technologies, Vancouver, BC), supplemented with 100 ng/mL SCF, 10 ng/mL FLT3-L, 20 ng/mL IL-7, 10 ng/mL IL-3, 20 ng/mL IL-6, 20 ng/mL G-CSF, 20 ng/mL GM-CSF. Cells were supplemented with 100 μg/ml penicillin, and 100 U/ml streptomycin, at 37° C. and 5% CO₂ in humidified atmosphere. The University Health Network (Toronto, ON) and MD Anderson Cancer Center (Houston, Tex.) institutional review boards approved the collection and use of human tissue for this study. All samples were obtained from consenting patients.

Cell Viability Assays.

For Alamar-Blue assays, cells (1×10⁴/well) were plated in 96-well plates (final volume of 100 μL/well), and treated with increasing concentrations of ONC201 and ONC212 (0 to 100 μM). After a 72-h period of incubation at 37° C., 10 μL of Alamar Blue was added to the culture medium and the mixture was incubated for an additional 2 h at 37° C. Cytotoxicity was measured using spectrophotometry of fluorescence at excitation 560 nm & emission 590 nm (SpectraMax M3, Molecular Devices, San Jose, Calif.). For apoptosis analysis, annexin V and PI binding assays were performed to assess apoptosis as described previously (Ishizawa et al., 2016). Cells (1.5×10⁵/well for AML cells in 24-well plates and 0.8×10⁵ for HCT116 cells in 12-well plates) were plated and treated with ONC201 and ONC212. Annexin V and PI were stained after 72 h incubation. Annexin V- and PI-negative cells were counted as live cells.

Cellular Thermal Shift Assay (CETSA).

CETSA was conducted as previously described (Jafari et al., 2014). OCI-AML2 cells were treated with increasing concentrations of ONC201 or ONC212 for 30 min at 37° C. Cells were then washed and re-suspended in PBS containing proteinase inhibitors and heated to 67° C. for 3 min using a thermal cycler (SimpliAmp, Applied Biosystems). This temperature was experimentally derived by heating cells pretreated with the drug for 1 h at different temperatures to determine the optimal thermal shift of the protein. Following this step, cells were lysed by four freeze-thaw cycles with vortexing, and pure cell lysates were collected after centrifugation at 16,000 g for 30 min at 4° C.

In wash-off experiments, ONC201 (10 μM) treated cells were washed in PBS, pelleted and re-suspended in fresh media and incubated for increasing time intervals starting from 15-75 min at 37° C. After this, cells were again washed and re-suspended in PBS containing proteinase inhibitors, heated to 67° C. for 3 min, and cell lysates were collected as described above.

RNA-Sequencing.

Barcoded, Illumina compatible, strand-specific total RNA libraries were prepared using the TruSeq Stranded Total RNA Sample Preparation Kit (Illumina, San Diego, Calif.). Briefly 1 μg of DNase I treated total RNA was depleted of cytoplasmic and mitochondrial ribosomal RNA (rRNA) using Ribo-Zero Gold (Illumina). After purification, the RNA was fragmented using divalent cations and double stranded cDNA was synthesized using random primers. The ends of the resulting double stranded cDNA fragments were repaired, 5′-phosphorylated, 3′-A tailed and Illumina-specific indexed adapters were ligated. The products were purified and enriched by 11 cycles of PCR to create the final cDNA library. The libraries were quantified using the Qubit dsDNA HS Assay Kit (ThermoFisher) and assessed for size distribution using the Fragment Analyzer (Advanced Analytical, Ankeny, Iowa), then multiplexed 4 libraries per pool. Library pools were quantified by qPCR and sequenced, one pool per lane, on the Illumina HiSeq4000 sequencer using the 75 bp paired end format. For each sample, TopHat was used to align reads from FASTQ files to the reference genome (hg19) and generate BAM files. These were then used as input to rnasegmut, which identifies genomic nucleotide positions at which a minimum number and proportion of reads have a variant sequence, i.e., indels or single-nucleotide variants (SNVs). There was no filtering to exclude known single-nucleotide polymorphisms (SNPs). For each SNV identified in either or both of the parental or ONC201-resistant samples of Z138 cells, rnasegmut provided the number of reads (forward and backward) with a WT nucleotide in that position, and the number of reads with the SNV in that position, for each sample. If the total number of reads at that position exceeded a minimum number of total reads (20), Fisher's exact test was used to compare the difference in the mutant allele frequency (MAF) in parental vs. resistant cells. SNVs meeting the criteria for minimum read number and Fisher test-significant MAF difference in either direction (i.e., higher in either the drug-naïve or resistant cells) were further characterized by ANNOVAR (Wang et al., 2010) as to whether they were intergenic, intronic, in the 5′ or 3′ UTR, or within exons, and if the latter, whether they were synonymous (silent), nonsynonymous (NSV), or involved the gain or loss of a stop codon. All raw data have been deposited at the Sequence Read Archive (SRA), accession ID #SUB4176298.

Site Directed Mutagenesis.

All point mutations were induced using Phusion High Fidelity DNA polymerase or QuikChange II site directed mutagenesis kit (Agilent Technologies, Santa Clara, Calif.) using the manufacture's protocol (New England Biolabs, Ipswich, Mass.). The primers used were as follows:

Y118ACLPP fwd:
5′-gagagcaacaagaagcccatccacatggccatcaacagccctggtg
gtgtggtgacc-3′
Y118ACLPP rev:
5′-ggtcaccacaccaccagggctgttgatggccatgtggatgggcttc
ttgttgctctc-3′
D190ACLPP fwd:
5′-ggccaagccacagccattgccatccagg-3′
D190ACLPP rev:
5′-cctggatggcaatggctgtggcttggcc-3′

For in vitro experiments, mutant genes without mitochondrial targeting sequence (MTS) were fused in frame with N-terminal His6-SUMO-2 tags in pETSUMO2 expression vectors. For experiments involving mammalian cells lines, full-length mutant genes (with MTS) were cloned into an expression vector with a C-terminal VA-tag (StrepIII-His6-TEV-TEV-3×FLAG). All mutations were confirmed by sequencing.

Immunoblot Analysis.

Cells were lysed at a density of 1×10⁶/50 μL (for AML cells) or 1×10⁶/100 μL (for HCT116 cells) in protein lysis buffer (0.25 M Tris-HCl, 2% sodium dodecylsulfate, 4% β-mercaptoethanol, 10% glycerol, 0.02% bromophenol blue). Protein lysates for Oxphos cocktail antibodies were incubated for 30 min at room temperature, otherwise, at 95° C. for 5 min for denaturing (antibodies used are listed below). Immunoblot analysis was performed as reported previously (Ishizawa et al., 2016). Briefly, an equal amount of protein lysate was loaded onto a 10-12% SDS-PAGE gel (Bio-Rad), and quantitated using the Odyssey imaging system (LI-COR Biotechnology, Lincoln, Nebr.). Antibodies used: total OXPHOS rodent WB antibody cocktail, anti-SDHA, anti-SDHB, anti-NDUFA12, anti-ClpP, anti-ATF4, anti-eIF2α, anti-phospho-eIF2α (S51), anti-ClpP, anti-CQCRC2, anti-CS, anti-NDUFB8, anti-β-actin, and anti-GAPDH.

Proximity-Dependent Biotin Labeling (BioID).

Wild-type and Y118A mutant CLPP sequences were PCR amplified and fused in-frame with a mutant E. coli biotin conjugating enzyme, BirA R118G (or BirA*), in a pcDNA5 FRT/TO plasmid under a CMV promoter positively regulated by tetracycline. For each construct, in-frame fusion was confirmed by Sanger Sequencing. The plasmids were then transfected into T-REx 293 cells using PolyJet (3 μL) (SignaGen, Rockville, Md.). Stable cells expressing the tetracycline-regulated, BirA*-tagged WT or constitutively active mutant ClpP proteins were selected using hygromycin B (200 μg/mL). Cell pools expressing the BirA* epitope tag alone, or BirA* fused to the unrelated mitochondrial enzyme ornithine transcarbamoylase (OTC) were used as negative controls.

At approximately 60% confluence, cells were treated with 1 μg/mL tetracycline and 50 μM biotin in addition to 0.6 μM ONC201 or vehicle control for 48 h. Cells were scraped in their media, pooled and washed twice in 25 mL cold PBS, pelleted by centrifugation at 1000×g for 5 min at 4° C., and lysed in ice-cold modified RIPA buffer for 1. Pure cell lysates were then incubated with RIPA-equilibrated streptavidin-sepharose beads (GE Healthcare, Little Chalfont, UK) in an end-over-end rotator for 2 h at 4° C. Beads were washed seven times with 1 mL of 50 mM ammonium bicarbonate (pH 8.0) and the biotinylated proteins were digested with trypsin. Two separate biological replicates (starting from the cloning phase) were generated for wild-type ClpP (treated and untreated) and each mutant. Samples containing the peptide fragments were analyzed by mass spectrometry.

Mass Spectrometry Analysis.

High performance liquid chromatography was conducted using a 2-cm pre-column (Acclaim PepMap™ 100; 75 μm ID; 3 μm, 100 Å C18; ThermoFisher Scientific, Waltham, Mass.) and a 50-cm analytical column (Acclaim® PepMap RSLC, 75 μm ID; 2 μm, 100 Å C18; ThermoFisher Scientific, Waltham, Mass.), applying a 120-min reversed-phase gradient (225 nL/min, 5-40% CH₃CN in 0.1% HCOOH) on an EASY-nLC1000 pump (ThermoFisher Scientific, Waltham, Mass.) in-line with a Q-Exactive HF mass spectrometer (ThermoFisher Scientific, Waltham, Mass.). A parent ion MS scan was performed at a resolution of 60,000 (FWHM at 200 m/z), followed by up to 20 MS/MS scans (15,000 FWHM resolution, minimum ion count of 1000 for activation) of the most intense MS scan ions using higher energy collision induced dissociation (HCD) fragmentation.

Dynamic exclusion was activated such that MS/MS of the same m/z (within a range of 10 ppm; exclusion list size=500) detected twice within 5 sec was excluded from analysis for 15 sec. For protein identification, Thermo RAW files were converted to the .mzML format using Proteowizard (Kessner et al., 2008), then searched using X!Tandem (Craig and Beavis, 2004) and Comet (Eng et al., 2013) against the Human RefSeq Version 45 database (containing 36113 entries). Search parameters specified a parent ion mass tolerance of 10 ppm, and an MS/MS fragment ion tolerance of 0.4 Da, with up to 2 missed cleavages allowed for trypsin. Variable modifications of +16@M and W, +32@M and W, +42@N-terminus, and +1@N and Q were allowed. Proteins identified with an iProphet cut-off of 0.9 (corresponding to ≤1% FDR) and at least two unique peptides were analyzed with SAINT Express v.3.6. Control runs (18 runs from cells expressing the FlagBirA* epitope tag only) were collapsed to the two highest spectral counts for each prey, and high confidence interactors were defined as those with BFDR≤0.01. All raw mass spectrometry files have been deposited at the MassIVE archive (massive.ucsd.edu), accession ID #MSV000082381.

Network Analysis.

ClpP interaction data were imported into Cytoscape 3.6.0, and proteins grouped according to previously reported physical interaction and functional data.

Lentiviral Infection and CpP Over-Expression.

A lentiviral wild-type or D190A mutant ClpP-over-expressing vector was generated by amplifying the cDNA by using primers CLPP cDNA fwd and CLPP cDNA rev (listed below) from Z138 cells and inserting it by InFusion cloning (TaKaRa Bio USA, Mountain View, Calif.) between the EcoR1 and BamH1 sites of pCDH-EF1a-MCS-BGH-PGK-GFP-T2A-Puro (Systems Biosciences, Palo Alto, Calif.) by using primers InFusion CLPP fwd and InFusion CLPP rev (listed below). Then, CLPP D190A was derived from the wild type vector using paired primers (CLPP mut D190A fwd and CLPP mut D190A rev) (listed below) with a QuikChange II site directed mutagenesis kit (Agilent Technologies, Santa Clara, Calif.). The manufacturer's method was followed except that used Stbl3 cells (ThermoFisher, Waltham, Mass.) were used in lieu of XL10-Gold. The correct clones were identified by Sanger sequence analysis. The sequences of all primers used to construct plasmids are listed below:

CLPP cDNA fwd:
(SEQ ID NO: 1)
5′-ACTGAATTCGCCACCATGTGGCCCGGAATATTGGT-3′
CLPP cDNA rev:
(SEQ ID NO: 2)
5′-ATCGGATCCTCTCAGGTGCTAGCTGGGAC-3′
InFusion CLPP fwd:
(SEQ ID NO: 3)
5′-TAGAGCTAGCGAATTGCCACCATGTGGCCCGGAATATT-3′
InFusion CLPP rev:
(SEQ ID NO: 4)
5′-CGGCGGCCGCGGATCTCAGGTGCTAGCTGGGACAG-3′
CLPP mut D190A fwd:
(SEQ ID NO: 5)
5′-GGGCCAAGCCACAGCCATTGCCATCCAGGCAG-3′
CLPP mut D190A rev:
(SEQ ID NO: 6)
5′-CTGCCTGGATGGCAATGGCTGTGGCTTGGCCC-3′
CLPP1 890 rev seq:
(SEQ ID NO: 7)
5′-GGCTCATCCTCACCGTCCTG-3′
CLPP1 540 rev seq:
(SEQ ID NO: 8)
5′-GATGTACTGCATCGTGTCGT-3′

A tetracycline-inducible system based on two lentiviral vectors was developed as previously described (Frolova et al., 2012). The first lentiviral vector (pCD510-rtTA) was generated by excising the reverse tetracycline-controlled transactivator (rtTA) coding sequence from pSLIK-Venus-TmiR-Luc (ATCC ID: MBA-239) with BamHI and BstBI and cloning the resulting fragment into NotI and BstBI restriction sites of pCD510-B1 (SystemBio). Thus, pCD510-rtTA expresses rtTA under the CMV promoter and Puromycin selection marker under a second promoter EF-1. To generate the second vector (pCD550A1-TRE), the original EF1 promoter was replaced by an inducible promoter composed of six tetracycline-responsive elements (TRE) followed by the minimal CMV promoter. cDNA sequence of wild-type or Y118A mutant CLPP was inserted under the control of a tetracycline inducible promoter (TRE) followed by the minimal CMV promoter and CopGFP, as a selection marker, under the control of the EF-1 promoter. For lentiviral infections, HEK293T cells (ATCC, Manassas, Va.) were co-transfected with pMD2.G and psPAX2 (kind gifts of Didier Trono, plasmids 12259 and 12260, respectively, Addgene Inc., Cambridge, Mass.) along with the lentiviral vectors using JetPrime transfection reagent (VWR, Radnor, Pa.) according to the manufacturer's protocol. The transfection medium was replaced after 6 h with fresh DMEM medium with 10% FBS and 24 h later the viral supernatants were collected and concentrated by using Centricon Plus-70 filter units (Sigma-Aldrich). OCI-AML3, Z138, and HCT116 cells were infected overnight with viral supernatants supplemented with 8 μg/mL of Polybrene (Sigma-Aldrich). Seventy-two hours after infection, stably transduced cells were selected by FACS resulting in a homogeneous population of GFP-labeled cells.

Measurement of Oxygen Consumption Rate.

Oxygen consumption was measured using a Seahorse XF96 analyzer (Seahorse Bioscience, North Billerica, Mass.). Cells were treated with increasing concentrations of ONC201 or vehicle control (DMSO) in their growth medium for 72 h at 37° C., resuspended in XF Assay medium supplemented with 2.0 g/L glucose and 100 mM pyruvate, and seeded at 1×10⁵ cells/well in XF96 plates. Cells were then equilibrated to the un-buffered medium for 60 min at 37° C. in a CO₂-free incubator and transferred to the XF96 analyzer. To measure the spare reserve capacity of mitochondrial respiratory chains, cells were treated with 2 μM oligomycin and 0.25 μM carbonyl cyanide p-trifluoromethoxyphenylhydrazone (FCCP) in succession.

Respiratory chain complexes activity. Enzymatic activities of respiratory chain complexes were measured as previously described (Sriskanthadevan et al., 2015). NADH-dependent activity of complex I was determined using Complex I Enzyme Activity Microplate Assay Kit in whole cell lysates following oxidation of NADH to NAD+ and simultaneous reduction of the provided dye. Complex II (succinate dehydrogenase) activity was measured in 2 μg isolated mitochondria in 20 mM sodium succinate-supplemented 100 mM HEPES, pH 7.4 containing 1 mg/mL bovine serum albumin, 20 μM rotenone, and 2 mM KCN by monitoring malonate-sensitive reduction of 170 μM 2,6-dichloroindophenol when coupled to complex II-catalyzed reduction of 50 μM decylubiquinone (Skrtic et al., 2011). Complex IV activity was measured by KCN-sensitive oxidation of 2 mg/mL ferrocytochrome c in 3 μg isolated mitochondria treated with 1 mg/mL dodecyl-D-maltoside in 25 mM Tris buffer, pH 7.0 supplemented with 125 mM KCl. Ferrocytochrome c was obtained by reduction of 40 mg/mL ferricytochrome c with 0.5 M L-ascorbic acid (Skrtic et al., 2011).

Mitochondrial ROS Measurement.

To measure reactive oxygen species level in mitochondrial, cells were treated with ONC201 (0-2.5 μM) for 72 h at 37° C., stained with MitoSox (Molecular Probes/Life Technologies, Eugene, Oreg.), and incubated in the dark for 30 min at 37° C. and 5% CO₂ in humidified atmosphere. Cells were then centrifuged to remove the dye and resuspended in binding buffer containing annexin V-FITC (BioVision, Milpitas, Calif.). Following this step, annexin V negative cells were identified and analyzed by flow cytometry in a Canto II 96 well cytometer (Fortessa system, Becton Dickinson, San Jose, Calif.). Positive control samples were treated with 50 μM antimycin A (Sigma-Aldrich) at 37° C. for 5 h before staining with MitoSox.

Quantification and Statistical Analysis.

Statistical analyses were performed using the two-tailed Student's t-test, One-way ANOVA, or Mann-Whitney test by the Prism (version 7.0; GraphPad Software) statistical software programs. The Kaplan-Meier method was used to generate survival curves, and log-rank test was used for comparison of the two groups. P-values less than 0.05 were considered statistically significant (*P<0.05, **P<0.01, ***P<0.001, ****P<0.0001). Unless otherwise indicated, values are expressed as the mean±SD calculated by performing three independent experiments.

Data and Software Availability.

The structure of the human mitochondrial ClpP in complex with ONC201 was deposited into the RCSB—Protein Data Bank (PDB) under the accession number 6DL7.

### Example 1—Activation of Mitochondrial ClpP Induces Anti-Tumor Effects In Vitro and In Vivo

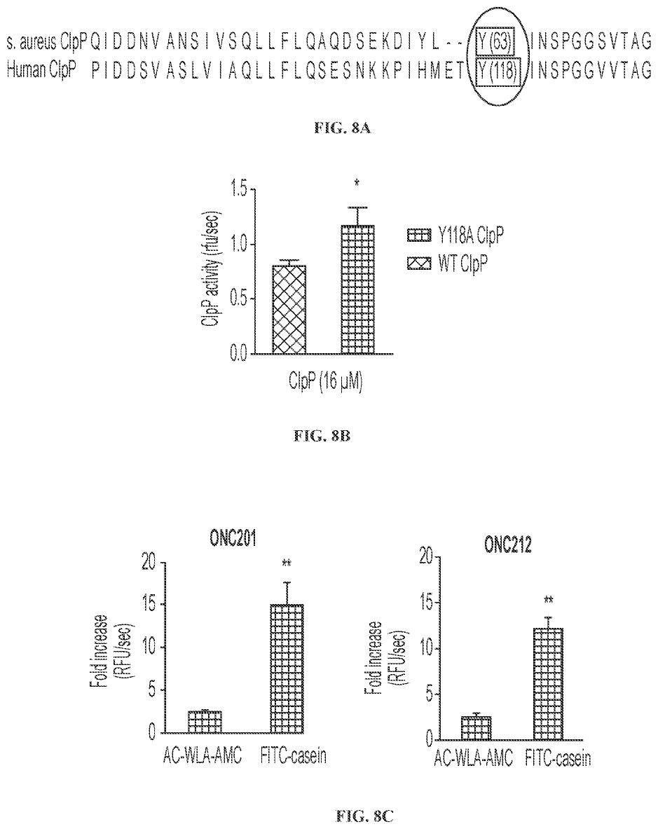

Activation of ClpP is cytotoxic to bacteria (Brotz-Oesterhelt et al., 2005; Conlon et al., 2013), so the anti-cancer effects of ClpP activation were tested by generating a constitutively active ClpP mutant by engineering a point mutation (Y118A) in human ClpP. This site was selected because it is homologous to the Y63A mutation in S. aureus ClpP (FIG. 8A). The Y63A ClpP mutation in S. aureus enlarges the entrance pores of the bacterial enzyme causing hyperactivation of the protease (Ni et al., 2016). Recombinant Y118A ClpP was purified and its enzymatic activity was tested. Compared to wild-type (WT) ClpP, Y118A ClpP demonstrated increased cleavage of its fluorogenic protein substrate FITC-casein in a cell-free enzymatic assay (Leung et al., 2011) (FIG. 8B).

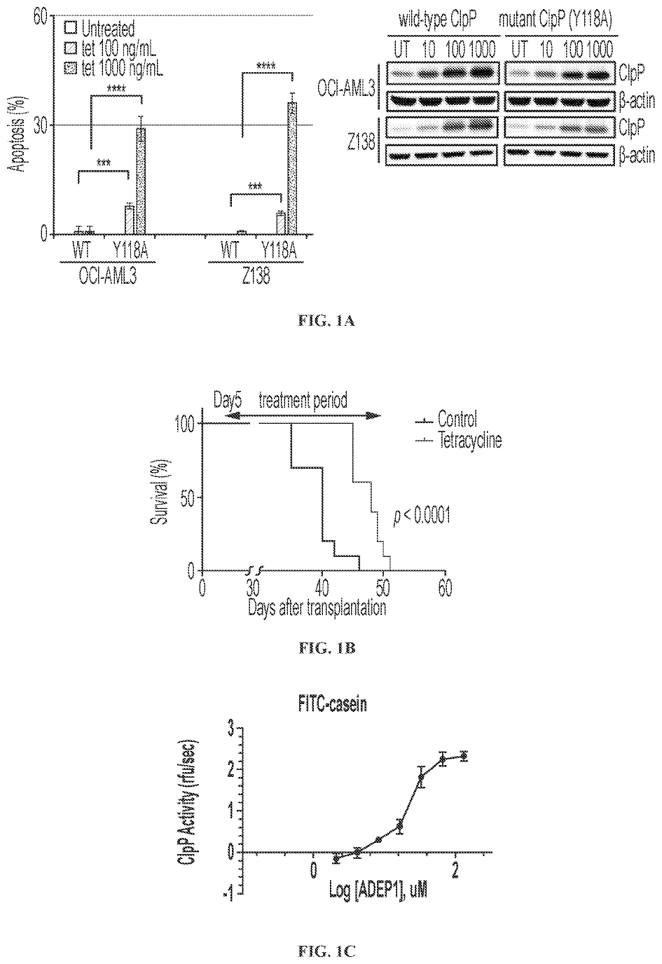

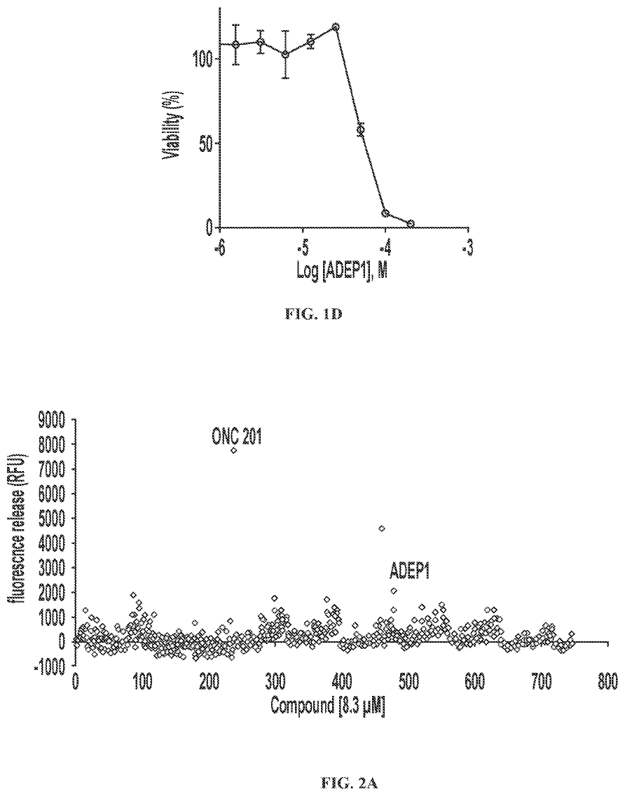

To evaluate the effects of this mutation in tumor cells, OCI-AML3 and Z138 cells were transduced with tetracycline-inducible WT or mutant ClpP (Y118A) via lentiviral infection and then treated with tetracycline to induce the expression. Induction of the constitutively active ClpP mutant, but not WT ClpP, induced apoptosis in a dose-dependent manner (FIG. 1A). The genetic activation of ClpP also exerted in vivo anti-tumor effects, consistent with its pro-apoptotic activity. Z138 cells with a tetracycline-inducible mutant ClpP (Y118A) were injected intravenously into NSG mice. Mice were then treated with tetracycline or vehicle. The tetracycline-treated group survived significantly longer than the untreated group (median survival: 48 vs 40 days, p<0.0001) (FIG. 1).

As an alternative strategy to test the antitumor effects of ClpP activation, Acyldepsipeptide 1 (ADEP1) was used. ADEP antibiotics are known activators of bacterial ClpP that bind the protease outside of its active site at the ClpX interface and open the ClpP axial pore. The effects of ADEP1 were tested on mitochondrial ClpP and it was demonstrated that it activated the mitochondrial protease and promoted ClpP cleavage of FITC-casein (EC₅₀ 21.33 μM [95% CI 20.12-22.61]) (FIG. 1C). OCI-AML2 cells were then treated with this compound and it was demonstrated that it reduced the growth and viability of these cells with an IC₅₀ of 50 μM [95% CI 48.4-51.6] (FIG. 1D). Thus, these data indicate that genetic or pharmacologic activation of mitochondrial ClpP could induce lethality in tumor cells in vitro and in vivo.

### Example 2—the Imipridones ONC201 and ONC212 Potently Activate Mitochondrial ClpP

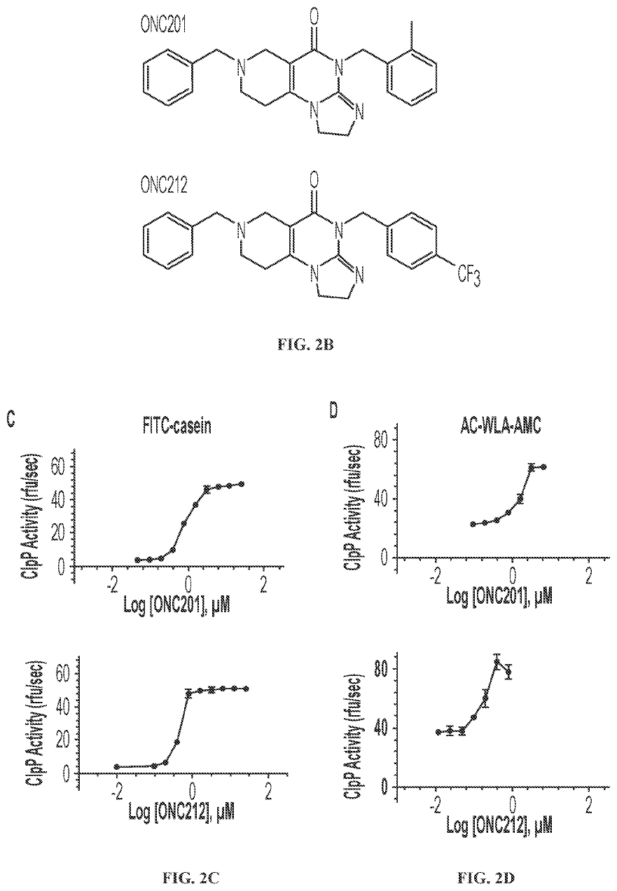

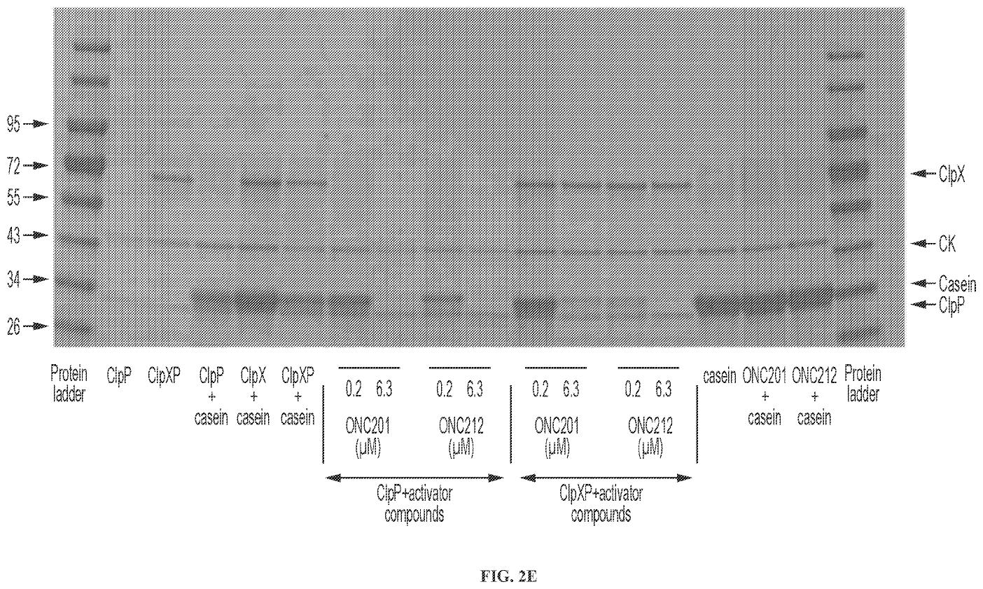

To find a more potent pharmacologic way to activate human ClpP, new small molecule ClpP activators were identified. Accordingly, a chemical screen was conducted of an in-house library of 747 molecules focused on on-patent and off-patent drugs approved for clinical use or in clinical trial for malignant and non-malignant indications. This library was screened to identify molecules that increased ClpP-mediated cleavage of its fluorogenic protein substrate FITC-casein using a cell-free enzymatic assay (Leung et al., 2011). Under basal conditions, ClpP could not cleave full-length proteins without its chaperone ClpX. However, the imipridone ONC201 activated the protease and facilitated ClpP-mediated cleavage of FITC-casein in the absence of ClpX (FIG. 2A). ONC201 (FIG. 2B) is a drug with preclinical efficacy in solid tumors and hematologic malignancies in vitro and in vivo (Allen et al., 2016; Allen et al., 2013; Ishizawa et al., 2016; Kline et al., 2016; Tu et al., 2017). The drug is currently being evaluated in clinical trials in a diverse spectrum of cancers (Arrillaga-Romany et al., 2017; Kline et al., 2016; Stein et al., 2017). Its more potent derivative, ONC212 (FIG. 2B), is in preclinical evaluation (Lev et al., 2017). Of note, molecular targets of imipridones that physically bind the drugs and are functionally important for its cytotoxicity have not been identified.

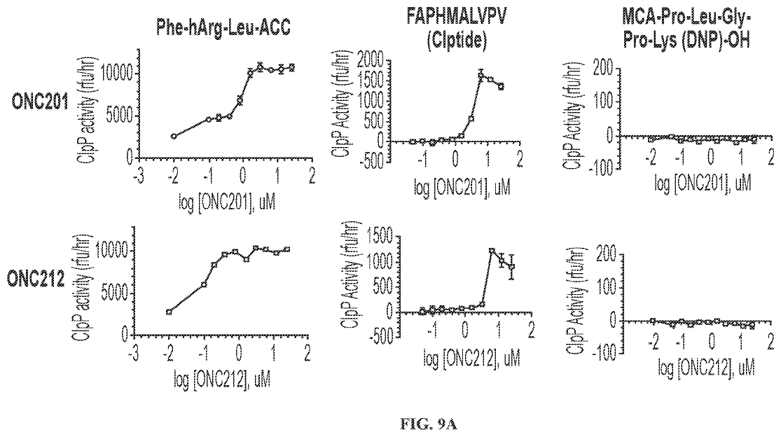

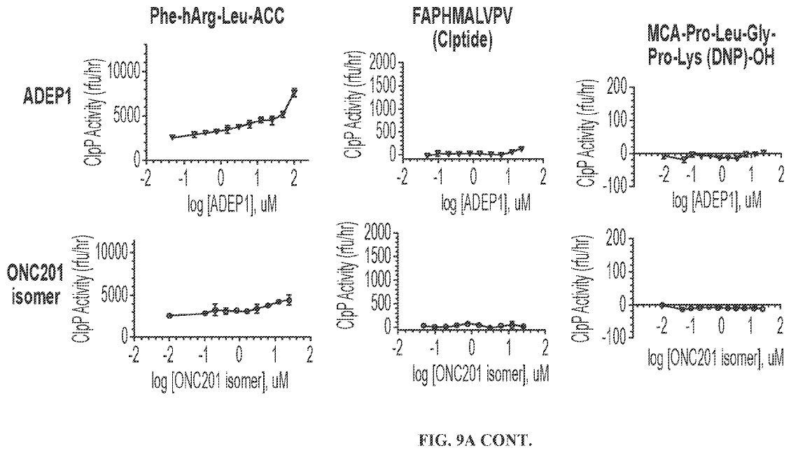

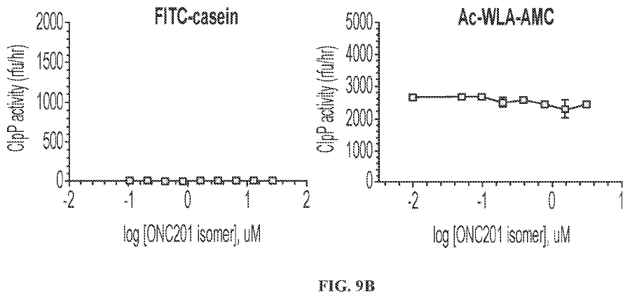

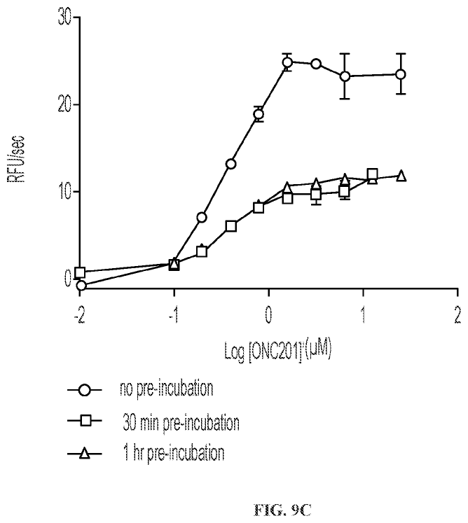

ONC201 activated ClpP without requiring ClpX and induced cleavage of FITC-casein as well as the fluorogenic peptides, AC-WLA-AMC, Ac-Phe-hArg-Leu-ACC, and FAPHMALVPC (Clptide) with EC₅₀s of 0.85 μM, 1.67 μM, 0.82 μM, and 3.23 μM, respectively, where the EC₅₀ represents the concentration of the drug that drives half maximal response (FIGS. 2C, 2D, and 9A). The effects of the structurally related imipridones, ONC201 inactive isomer (its inactive analog) and ONC212, and the bacterial ClpP activator, ADEP1, on ClpP activity were also tested. ONC212 increased ClpP-mediated cleavage of FITC-casein and AC-WLA-AMC, Ac-Phe-hArg-Leu-ACC, and FAPHMALVPC (Clptide) with EC₅₀s of 0.46 μM, 0.18 μM, 0.37 μM, and 3.37 μM, respectively (FIGS. 2C, 2D, and 9A). ADEP1 was a less potent ClpP activator compared to ONC201 and ONC212 (FIG. 9A) and the inactive isomer of ONC201 did not increase ClpP mediated cleavage of its substrates (FIGS. 9A, 9B). FITC-casein data showed clear positive cooperativity (Gersch et al., 2015) with Hill coefficients of 1.98±0.16 for ONC201 and 4.98±0.47 for ONC212. Notably, the activities of imipridones were greater than the activation achieved by the Y118A mutation in ClpP (FIGS. 8B, 8C). Another fluorogenic peptide, a non-ClpP substrate, MCA-Pro-Leu-Gly-Pro-D-Lys (DNP)-OH peptide, which was not cleaved after activating ClpP with imipridones or ADEP1, was also tested (FIG. 9A). Of note, pre-incubation of ClpP with ONC201 and ONC212, did not increase the ability of the compounds to activate ClpP, suggesting a reversible (non-covalent) mode of activation (FIG. 9C). As the imipridones were much more potent ClpP activators compared to ADEP1 (FIG. 1C), subsequent studies focused on these compounds.

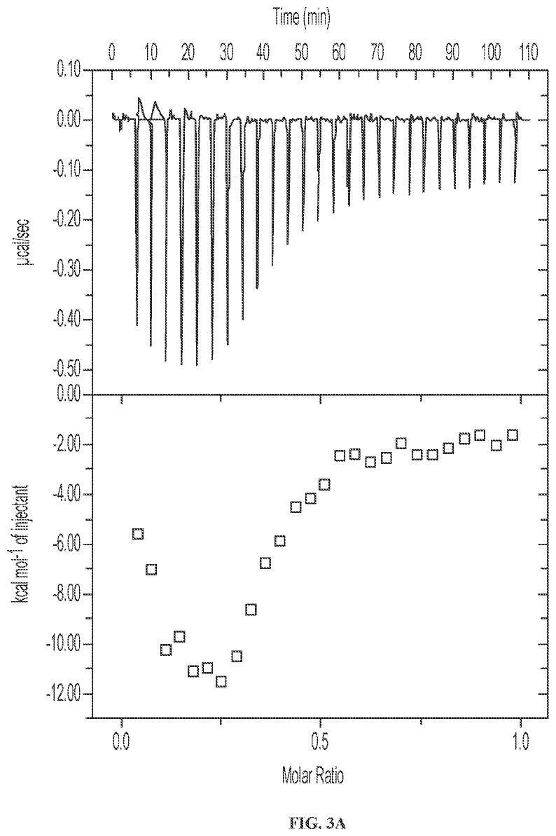

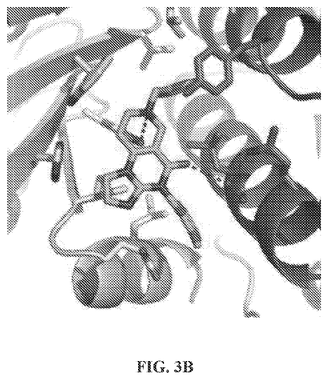

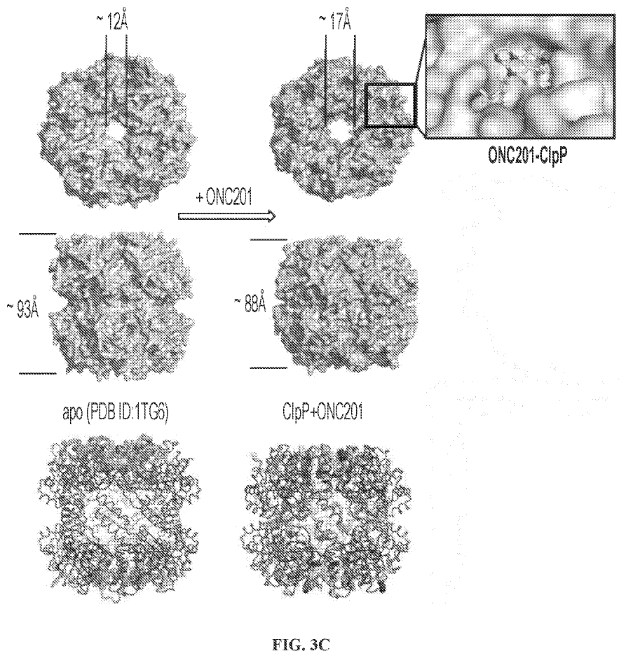

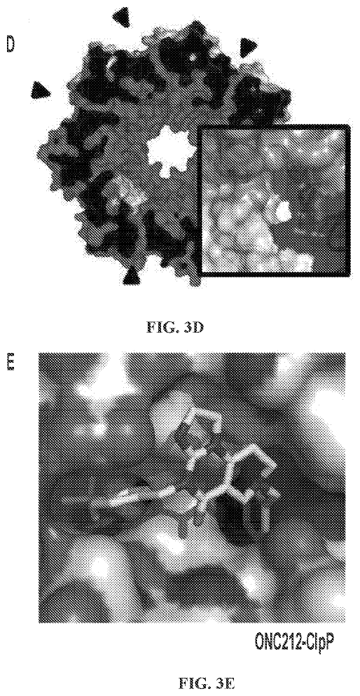

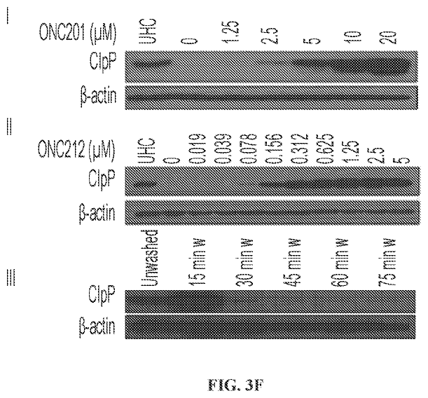

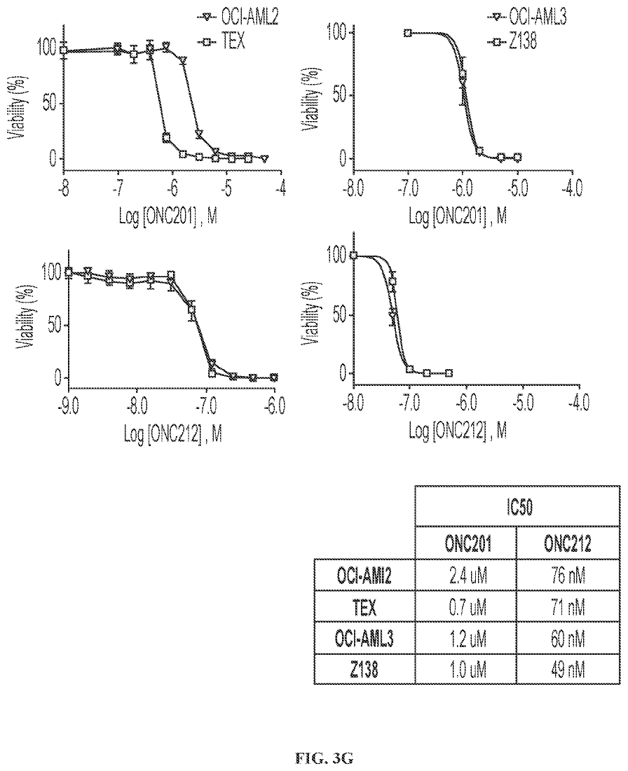

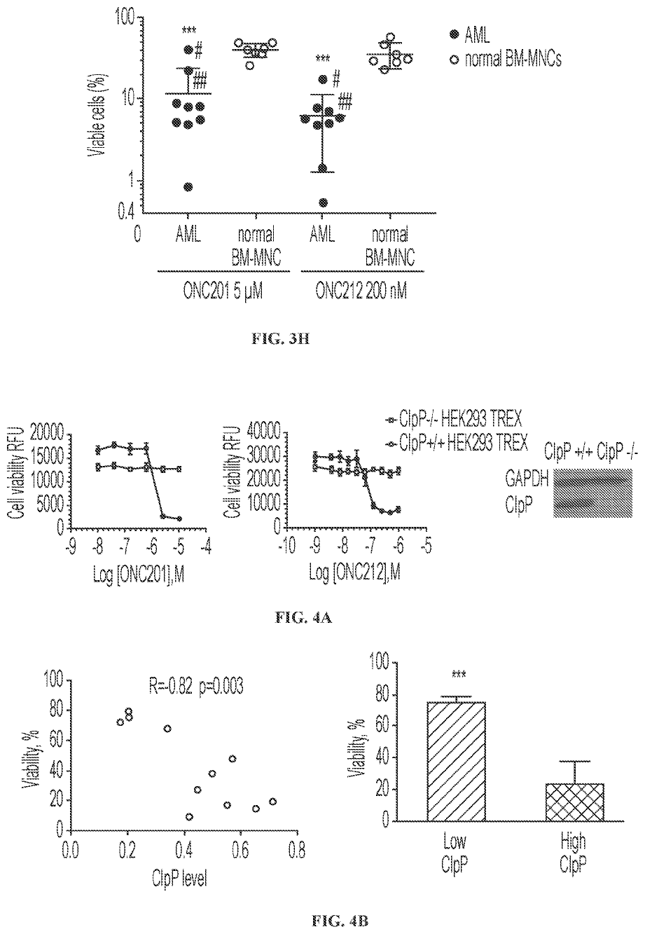

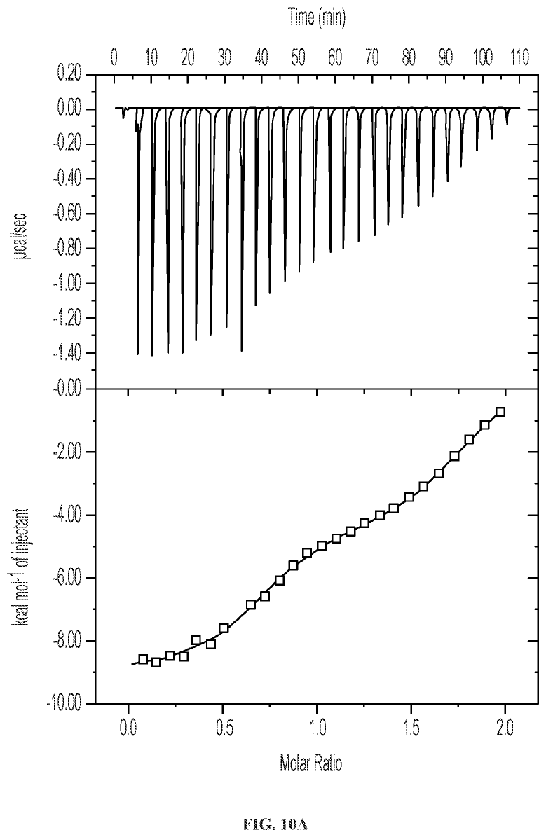

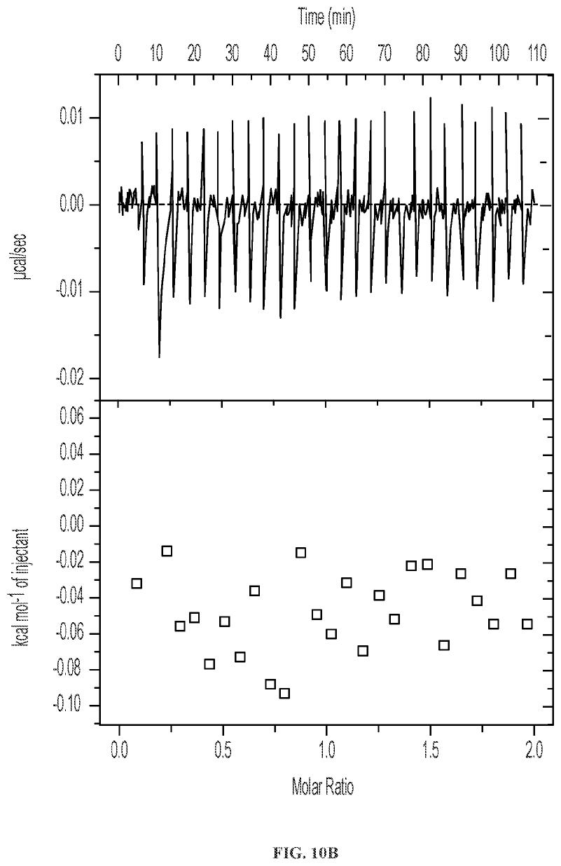

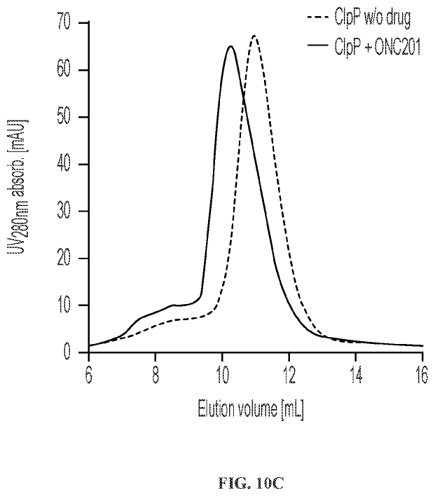

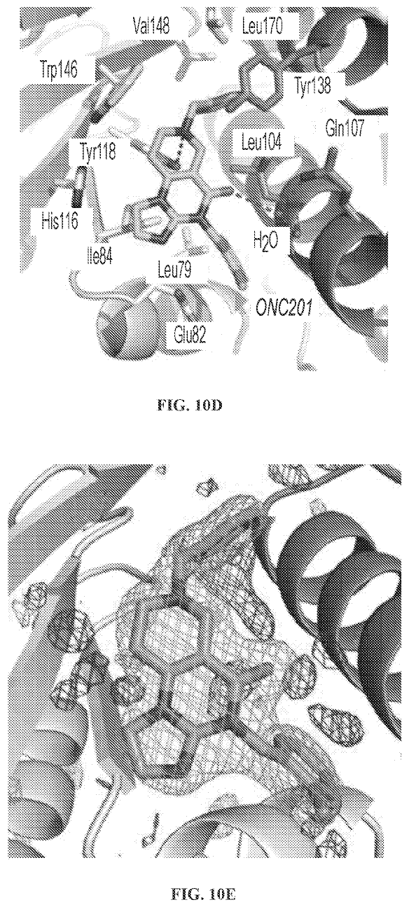

To confirm the results of the fluorogenic assays, the effects of ONC201 and ONC212 were tested in a gel-based assay that measures the degradation of α-casein by ClpP (FIG. 2E). The addition of ONC201 and ONC212 activated ClpP and induced cleavage of α-casein without the need for ClpX. It was then shown that ONC201 directly interacted with the recombinant protease using Isothermal Titration Calorimetry (ITC) by adding increasing amounts of ONC201 to a solution of ClpP (FIG. 3A), and in another setting, titrating ClpP into a solution of ONC201 (FIGS. 10A, 10B) (Gersch et al., 2015). Direct interaction of ONC201 with ClpP was also confirmed by gel filtration (FIG. 10C), where a clear shift towards higher molecular weight was observed. As human mitochondrial ClpP was shown, unlike bacterial ClpPs, to exist as heptamer in the absence of ClpX (Kang et al., 2005) even at concentrations >3 mg/mL, ONC201 binding to the protease clearly shifted the equilibrium from the 7-mer to the 14-mer of ClpP. Thus, taken together, ONC201 and its analogue, ONC212, were identified as ClpP ligands that bind and hyperactivate this mitochondrial protease.

### Example 3—ONC201 Binds ClpP Non-Covalently at the Interface with ClpX

To identify the precise molecular interaction between ONC201 and the ClpP protein, human ClpP protease was co-crystallized with the drug and the structure of the protein-drug complex was determined at 2 Å resolution (PDB-ID: 6DL7). Seven ONC201 molecules are clearly visible in the electron density map. They occupy hydrophobic pockets between each of the seven subunits (FIGS. 3B, 10D, and S10E). Direct interactions between protein residues and the ONC201 activator involve extensive hydrophobic contacts and a hydrogen bond to the hydroxyl group of Tyr-118 (2.8 Å) (FIG. 10D). In addition, the oxo-group of ONC201 forms water-mediated hydrogen bonds with the side chain nitrogen of Gln-107 and the carbonyl oxygen of Leu-104 (FIG. 10D). The phenyl ring of the drug is positioned between Tyr-138 and Tyr-118, engaging in π-stacking interactions.

The binding of ONC201 leads to the axial entrance pore opening up, increasing its radius from 12 Å, as seen in an apparently closed conformation of human mitochondrial ClpP (Kang et al., 2004), to 17 Å (FIG. 3C, top), doubling the pore size. The ClpP 14-mer assumes a more compact form and its height decreases from 93 Å to 88 Å (FIG. 3C, middle). In addition to opening the entrance pore, the N-terminal residues show increased dynamics, as evidenced by the significantly higher temperature factors of this region (FIG. 3C, bottom). Electron density corresponding to the first seven N-terminal residues is very weak and residues 64-73 lack any discernable density. The C-terminal residues following Pro-248 are also not visible in the electron density map. ONC201 binding induces further structural changes around the active site region at the heptamer-heptamer interface. In the human apo-ClpP structure (Kang et al., 2004), this region is well defined. In the ClpP-ONC201 complex, residues 178-193, encompassing the end of strand β6, all of strand β7, and the first third of helix α5, undergo a large conformational change and show increased dynamics with the region around residues 183-187 again not visible in electron density maps (FIG. 3C, bottom). This change directly impacts the placement of the catalytic triad residues (i.e., Ser-153, His-178, and Asp-227) in the active site. The ring of His-178 separates from Ser-153 by more than 5 Å while rotating by about 70°. Asp-227 moves in the same direction but only by 2.8 Å (FIG. 10F). It is worth noting that in the ONC201 complex the catalytic aspartates from subunits that are neighbors in the tetradecameric ring, across from each other at the heptamer-heptamer interface, approach each other rather closely (4.6 Å) whereas they are separated by ca. 17 Å in the ligand-free structure. In addition, it is now Ser-181, which is the closest interacting side chain, not the postulated catalytic Ser-153. The side chain hydroxyl of Ser-181 interacts closely (3.2 Å) with the carboxylate of Asp-227 in the neighboring subunit. The conformational changes induced by ONC201 binding also include the opening of channel-like pores in the central region of the “side wall” of the protease, similar to the ones described previously for the bacterial enzyme and represent potential escape routes for peptide products (Sprangers et al., 2005) (FIG. 3D). Thus, ONC201 binds ClpP non-covalently outside the active site, and activates the protease by stabilizing the ClpP 14-mer, enlarging the axial pore of the enzyme, and inducing structural changes in the residues surrounding and including the catalytic triad.

In ONC212, the 4-(2-methylbenzyl) group present in ONC201 is replaced by a 4-(4-trifluoromethylbenzyl) substituent. In the crystal structure of the ClpP-ONC201 complex, the ortho-methyl group of ONC201 points toward the bulk solvent. Its removal should only be of minor influence on its binding energy. In proteins, fluorophilic environments include peptide Ca multipolar interactions. Positively charged side chains of arginine residues also provide opportunities for binding enhancement (Muller et al., 2007).

When modeled based on the ONC201 site and subjected to two cycles of MD refinement (Adams et al., 2010), the para-trifluoromethyl substituent of ONC212 sticks into an extension of a generally apolar binding pocket of ClpP, which accepts the benzyl ring to which the CF3-group is connected (FIG. 3E). There are no strong clashes with protein residues and atomic movements observed are all distinctly smaller than 1 Å. The peptide bonds of Ile 75, Leu 79, Ala 101, and Phe 105 are in potential binding distance. In addition, the side chains of Arg 78 and Arg 81 are both close enough to be able to swing around and interact with the CF3-substituent. Arg 78, which forms a salt bridge with Glu 82, could easily be displaced in this interaction by Arg 81, especially as all three residues are on the protein surface and in contact with bulk solvent. Thus, the highly electronegative trifluoromethyl substituent likely enhances ONC212's potency by providing more opportunities for multipolar bonds and an improved structural complementarity to ClpP.

### Example 4—Imipridones Bind ClpP in Cells

Given the ability of ONC201 and ONC212 to activate ClpP in the cell-free assays above, it was tested whether they could bind ClpP in cells using Cellular Thermal Shift Assay (CETSA). CETSA evaluates ligand-induced changes in melting temperature (Tm) of target proteins in cells to determine the binding affinity of ligands towards their targets (Jafari et al., 2014). Both ONC201 and ONC212 bound endogenous ClpP in OCI-AML2 at concentrations associated with activation of the protease in the enzymatic assays. (FIGS. 3F (I & II) and 11A). Then, the reversibility of binding of ONC201 to ClpP was tested in OCI-AML2 cells by washing ONC201-treated cells in PBS and re-incubating them in fresh media prior to CETSA (FIG. 3E (III)). ClpP thermal stability rapidly decreased following removal of drug from the media, consistent with non-covalent binding observed in the crystal structures.

### Example 5—ClpP Activation by Imipridones ONC201 and ONC212 Kills Malignant Cells Through a ClpP-Dependent Mechanism

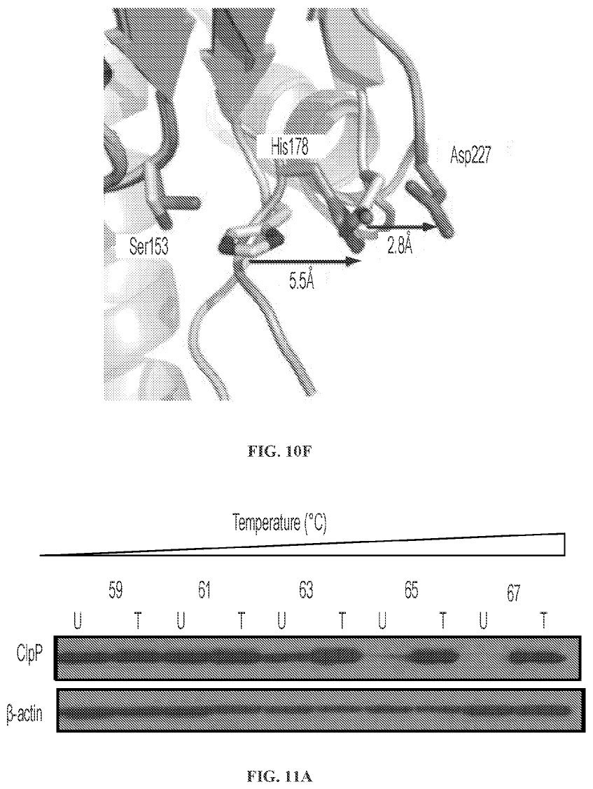

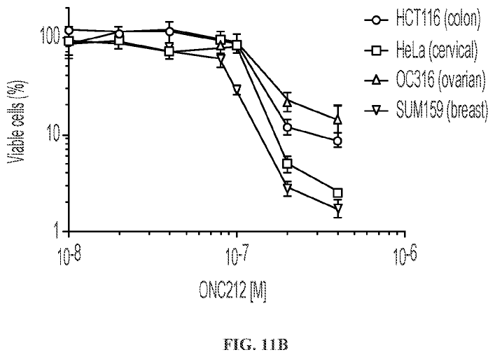

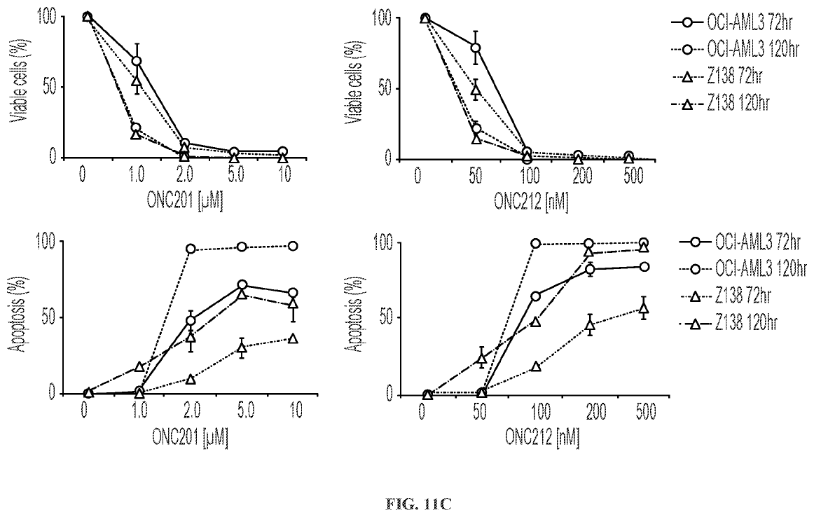

The effects of hyperactivating ClpP on the growth and viability of leukemia and lymphoma cells was further evaluated. OCI-AML2, OCI-AML3, TEX leukemia cells, Z138 lymphoma cells as well as HCT-116 (colon), HeLa (cervical), OC316 (ovarian), and SUM159 (breast) cells were treated with increasing concentrations of ONC201 and ONC212. Both ONC201 and ONC212 reduced the growth and viability of the tested cells with IC₅₀ values in the low micromolar (ONC201) or nanomolar range (ONC212) (FIGS. 3G and 11B). Cell death and apoptosis induction by the compounds was confirmed using the Annexin V/PI assay (FIGS. 3G and 11C). Reductions in growth and viability by the imipridones matched their ability to bind ClpP by CETSA and activate the enzyme in the enzymatic assays. The effects of ClpP activation were further assessed on primary AML and normal hematopoietic cells. ONC201 and ONC212 induced apoptosis in primary AML patient samples, including those with high-risk cytogenetics and molecular mutations (FIG. 3H & Table 3). Notably, profound efficacy of ONC201 in TP53 mutant tumors was recently reported (Ishizawa et al., 2016; Kline et al., 2016), an observation of potential clinical significance.

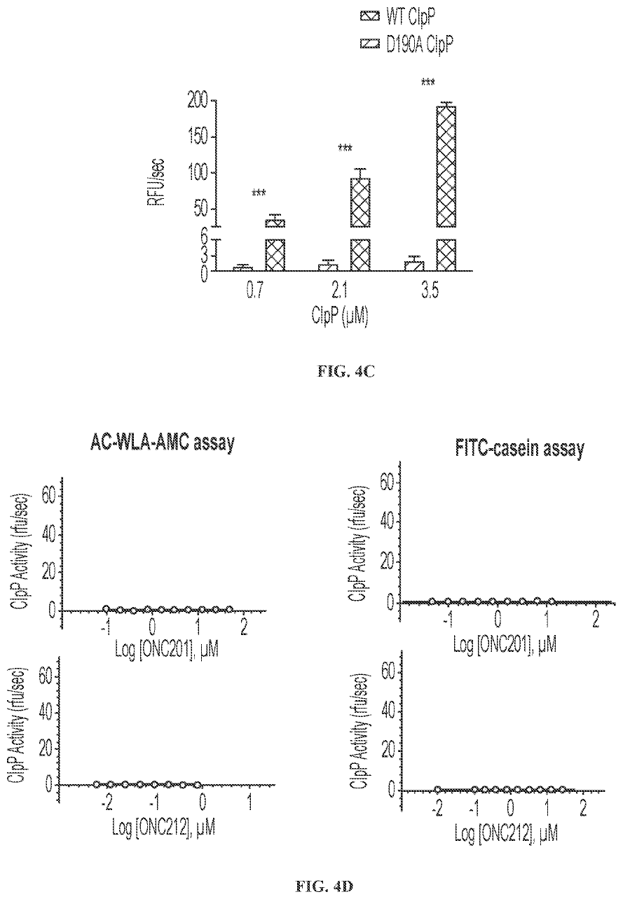

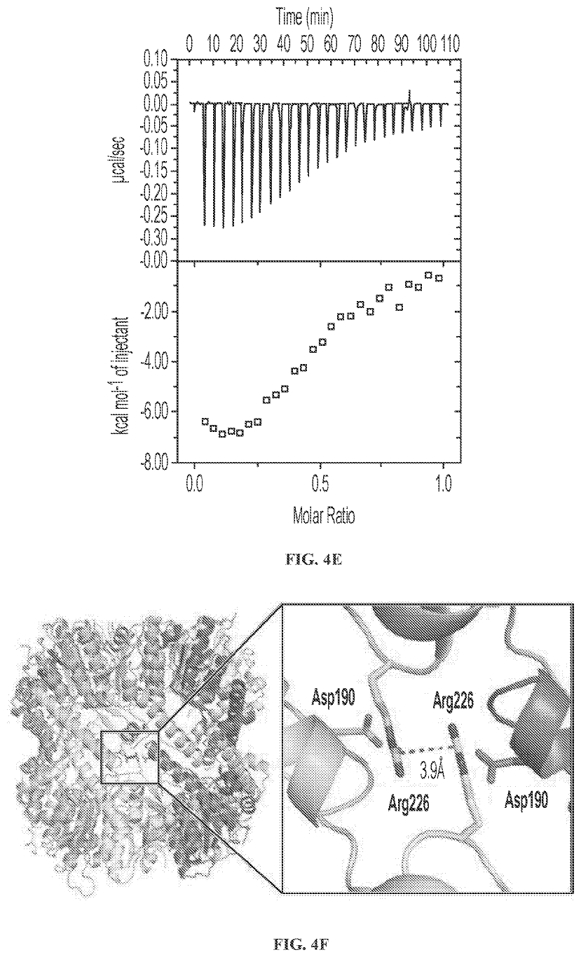

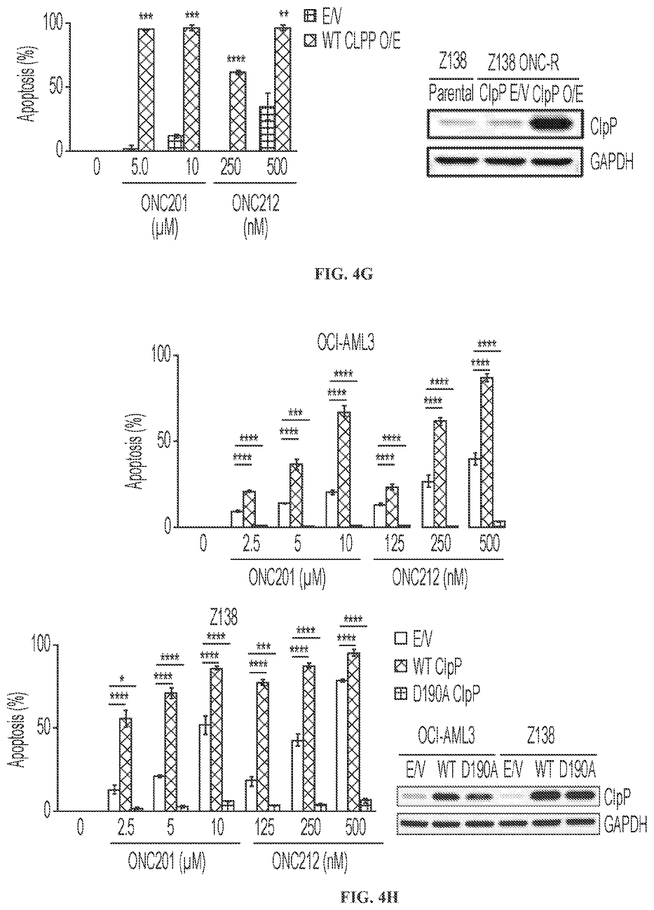

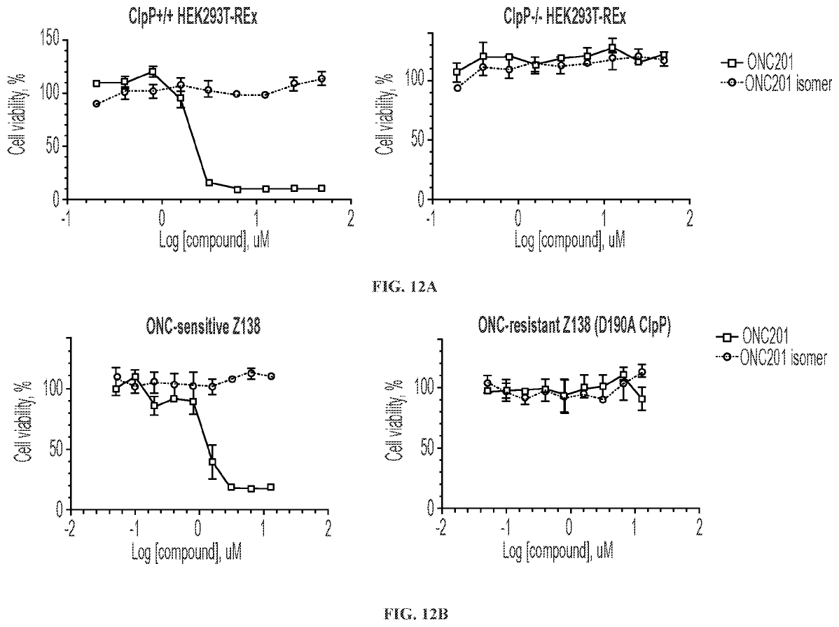

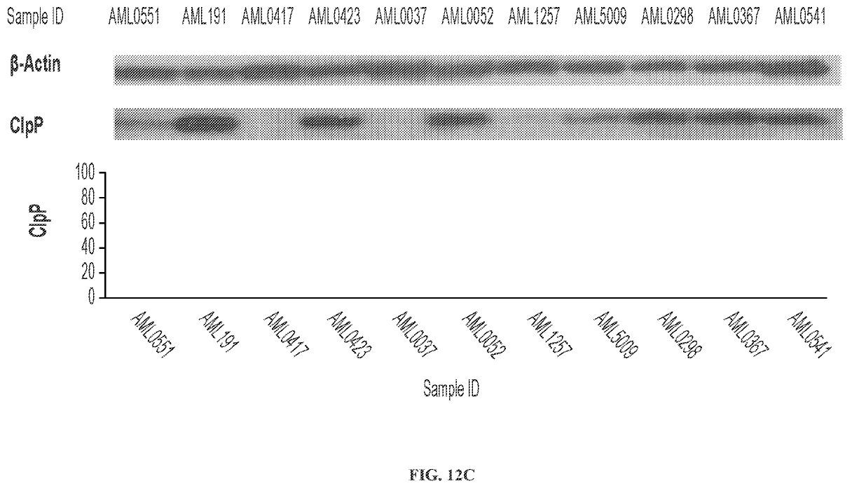

To assess whether activation of ClpP is functionally important for cell death induced by imipridones, CLPP+/+ and CLPP−/− T-REx HEK293 cells were treated with increasing concentrations of ONC201, ONC201 inactive isomer, and ONC212. ONC201 and ONC212 reduced the growth and viability of wild type cells, but CLPP−/− T-REx HEK293 cells that lack the protease were resistant to ONC201 and ONC212 (FIG. 4A). ONC201 isomer did not significantly decrease the growth and viability of CLPP+/+ or CLPP−/− T-REx HEK293, and ONC201-sensitive or ONC201-resistant Z138 cells (FIGS. 12A, 12B).

TABLE 3
Clinical information of samples used for FIG. 3H.

| Sample | | | | WBC | Blast | | | |
|---|---|---|---|---|---|---|---|---|
| No. | Gender | Age | Organ | (103/mm³) | (%) | Gene mutations | Cytogenetics | Disease Status |
| 1 | M | 68 | PB | 98.7 | 91 | FLT3-ITD, CEBPA, | intermediate | Refractory |
| | | | | | | WT1 | | |
| 2 (#) | M | 74 | PB | 8.9 | 66 | ASXL1, DNMT3A, | High risk (complex) | Refractory |
| | | | | | | IDH2, SRSF2, TP53 | | |
| 3 (##) | F | 36 | PB | 46.5 | 90 | DNMT3A, FLT3- | High risk (complex) | Relapsed/Refractory |
| | | | | | | ITD, NPM1, IDH2 | | |
| 4 | F | 41 | BM | — | 77 | PHF6 | High risk (complex) | Relapsed/Refractory |
| 5 | F | 51 | PB | 49.9 | 49 | NRAS, TET | Intermediate | Relapsed/Refractory |
| 6 | M | 72 | PB | 63.1 | 98 | FLT3, IDH1, NPM1, | Intermediate | Newly Diagnosed |
| | | | | | | PRPF40B, SRSF2 | (diploid) | |
| 7 | F | 62 | BM | — | 87 | KRAS, NRAS | Intermediate | Newly Diagnosed |
| 8 | M | 63 | BM | — | 77 | ASXL1, CSF3R, NF1 | High risk | Refractory |
| | | | | | | | (monosomy 7) | |
| 9 | M | 72 | BM | — | 90 | IDH2, NPM1, SRSF2 | intermediate | Newly Diagnosed |
| | | | | | | | (diploid) | |

#, ##: Samples which were relatively resistant to ONC201 in FIG. 2G.

TABLE 4
Clinical information of samples used for FIGS. 4B and 12C.

| | | | | | WBC | PB Blasts | BM Blasts | |
|---|---|---|---|---|---|---|---|---|
| ID | Age | Gender | Source | Disease Status | (×10e9/L) | (×10e9/L) | (%) | NPM1 |
| AML0367 | 42 | M | PB | Diagnosis | 313 | 291.09 | 90 | — |
| AML0551 | 32 | M | PB | Diagnosis | 145 | 101.5 | 85 | not done |
| AML1257 | 58 | M | PB | Diagnosis | 189 | 111 | 70 | Positive |
| AML0052 | 73 | F | PB | Diagnosis/Secondary | 60.7 | 0 | 20 | not done |
| | | | | to CMML | | | | |
| AML0298 | 54 | M | PB | Diagnosis | 97.7 | 89.88 | 97 | Positive |
| AML5009 | 73 | F | PB | Diagnosis/Secondary | 31.3 | 15.96 | 48 | Negative |
| | | | | to MPN | | | | |
| AML0541 | 51 | F | PB | Diagnosis | 166 | 151.3 | 86 | Positive |
| AML0037 | 24 | F | PB | Diagnosis | 43.3 | 12.99 | not done | not done |
| AML191 | 39 | M | BM | Diagnosis | 168 | 159.6 | 90 | not done |

### Example 6—Levels of ClpP are Associated with Response to ClpP Activators in Primary AML Cells

To identify whether ClpP expression levels in primary AML samples predicts their response to ClpP activators, pretreatment ClpP levels were measured in 11 primary AML samples and their response to ONC201 treatment was assessed. Sensitivity to ONC201 correlated with pretreatment ClpP expression in these samples (r=−0.82, p=0.003) (FIGS. 4B and 12C; Table 4). Primary AML patient samples with higher ClpP expression were significantly more sensitive to the ClpP activator compared with samples with pretreatment ClpP values that were 1 SD below average (P=0.0003). Thus, ClpP activators preferentially induce cell death and apoptosis in primary AML over normal cells and ClpP expression serves as a biomarker for patients that will respond to ClpP activators, including ONC201 and ONC212.

### Example 7—Inactivating Mutations in ClpP Render Cells Resistant to Imipridones

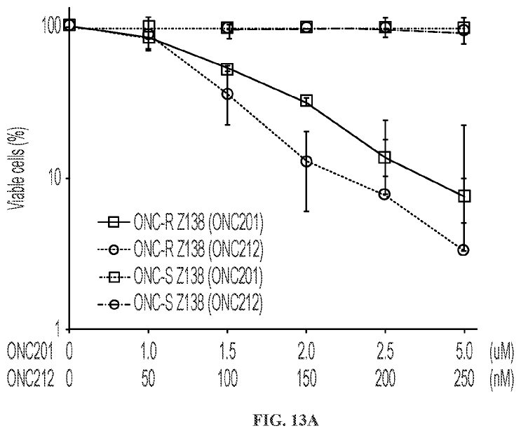

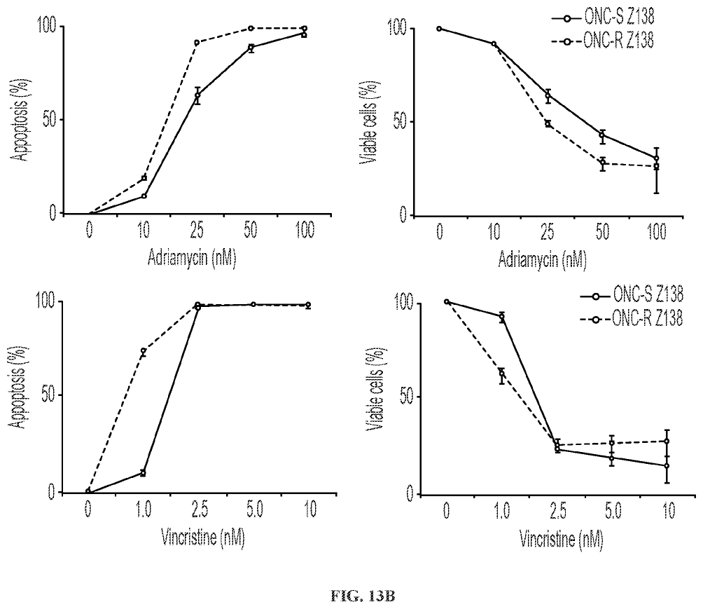

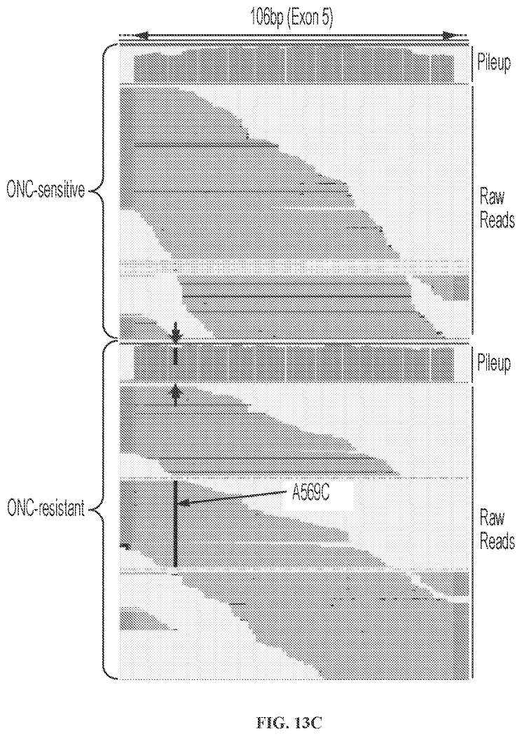

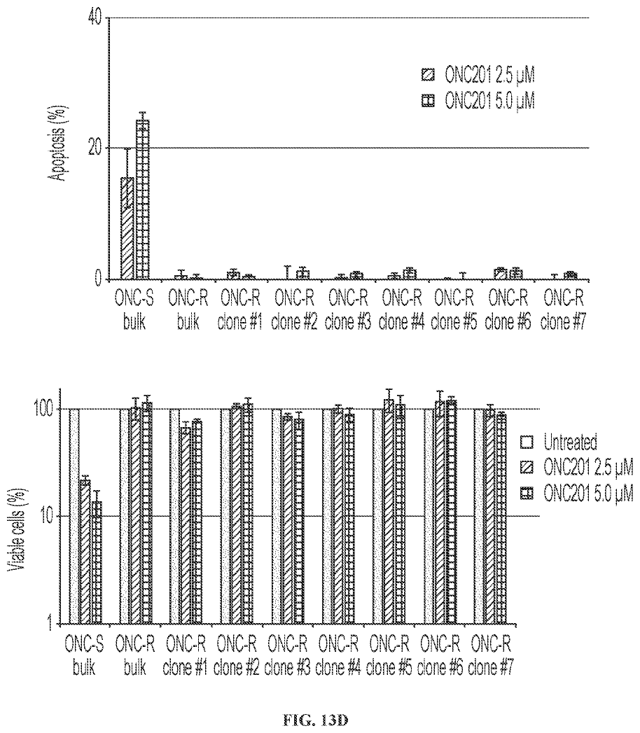

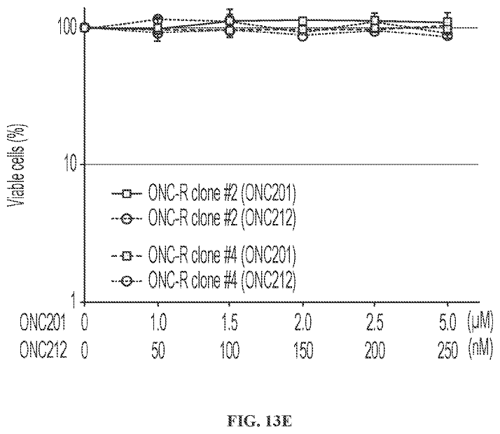

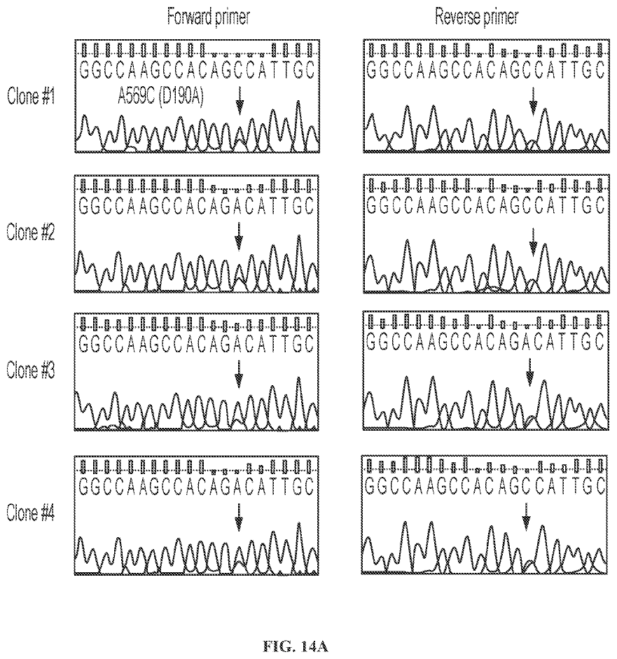

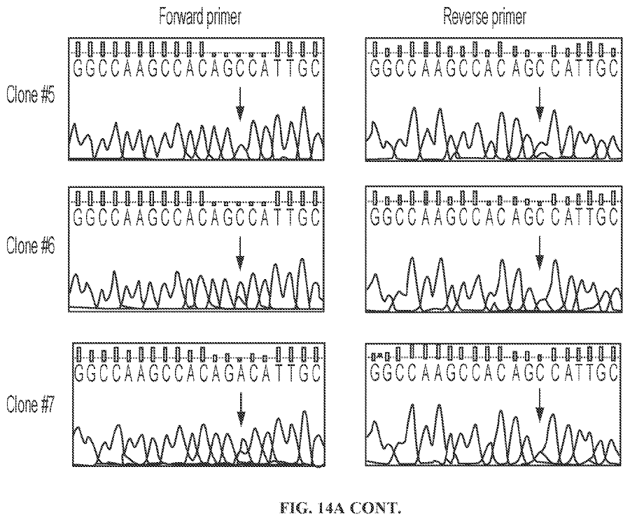

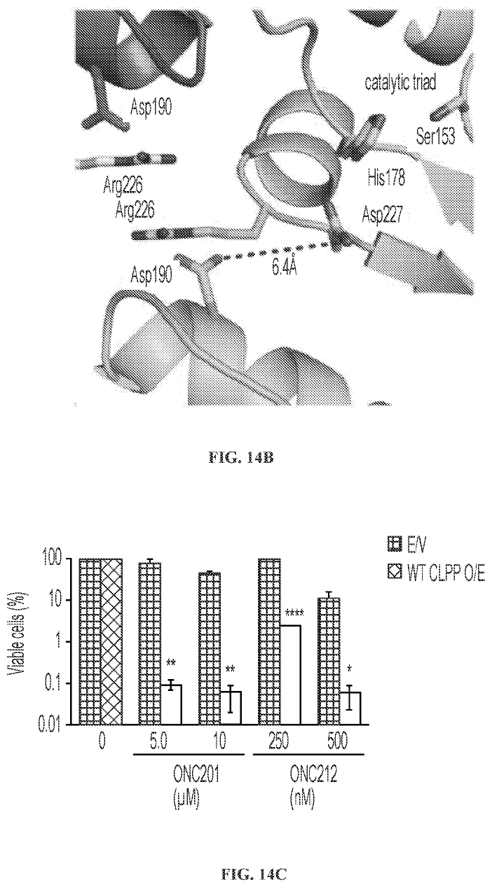

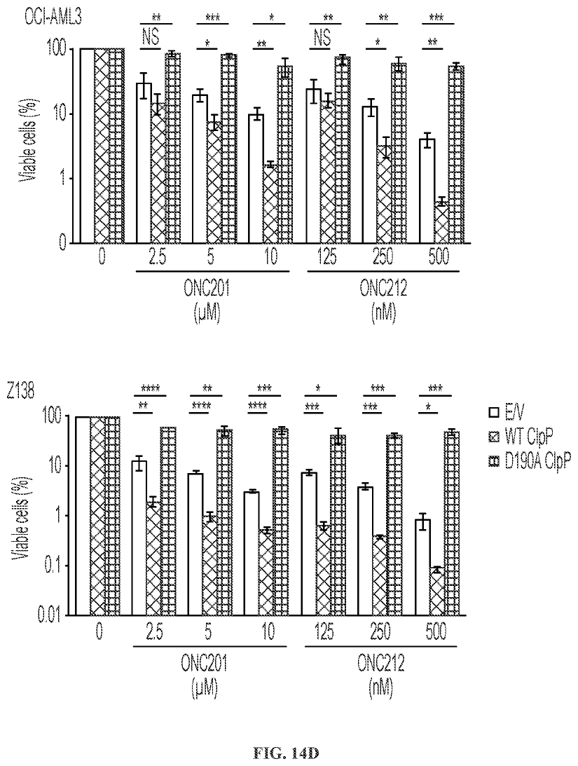

To further evaluate the importance of ClpP for ONC201 and ONC212 mediated cell death and identify potential mechanisms of resistance to ClpP activators, Z138 cells were treated with increasing concentrations of ONC201 and a population of cells resistant to the drug (ONC-R Z138) were selected. ONC-R Z138 cells were also cross-resistant to ONC212 (FIG. 13A), but retained similar sensitivity to Adriamycin and Vincristine (FIG. 13B). To identify the mechanism of resistance of these cells to ONC201 and ONC212, RNA sequencing (RNA-seq) was performed, and unbiased analysis identified the D190A mutation in ClpP (FIG. 13C) with an allele frequency of 47% in the ONC-R Z138 population of cells. To confirm the heterozygosity of the mutation, resistant clones were isolated and seven clones were randomly selected for analysis. All seven clones retained resistance to ONC201 and ONC212 (FIGS. 13D, 13E), and a heterozygous mutation in CLPP (D190A) was detected by Sanger sequencing of genomic DNA in every clone (FIG. 14A).

To assess how the D190A mutation affects ClpP function, recombinant D190A ClpP was generated and purified, and its enzymatic activity and response to ONC201 and ONC212 were measured. D190A ClpP had minimal proteolytic activity and could not degrade the fluorogenic peptide AC-WLA-AMC or FITC-casein under basal conditions (FIG. 4C). Moreover, ONC201 and ONC212 could not activate proteolytic activity of D190A ClpP for either peptide or protein substrates (FIG. 4D). However, ONC201 continued to bind recombinant D190A ClpP protease, as its binding site is a distance away from the mutation site, but the binding affinity was moderately reduced (FIG. 4E).

To understand how the D190A mutation might affect ClpP activity and structure, the crystal structures of human mitochondrial ClpP (Kang et al., 2004) and its ClpP-ONC201 complex were compared. In the former, Asp-190 is located at the dimer interface and is important as a compensating charge to an unusual stacked arginine pair that consists of Arg-226 residues from two neighboring peptide chains (FIG. 4F). Asp-190 is also only 6.4 Å from Asp-227 of the catalytic triad of ClpP (FIG. 14B). However, in the ClpP-ONC201 complex structure this region undergoes a major conformational change and displays high mobility with Asp-190 in close proximity to Asp-93 in most subunits. Loss of negative charge in D190A mutant can therefore have deleterious effects on the active site through impacting the mobility and sidechain interactions of the 178-193 loop.

In order to determine whether the D190A mutation was functionally important for resistance to ONC201 and ONC212, wild-type ClpP was overexpressed in D190A ClpP-mutant (ONC-R Z138) cells and D190A mutant ClpP in parental (wild-type ClpP) Z138 and OCI-AML3 cells. Over-expression of wild-type ClpP restored the sensitivity of the ONC-R Z138 cells to ONC201 and ONC212 (FIGS. 4G and 14C) while over-expression of D190A ClpP in parental Z138 and OCI-AML3 cells reproduced resistance to ONC201 and ONC212 (FIGS. 4H and 14D), suggesting dominant-negative inhibition of endogenous wild-type ClpP by the D190A mutant ClpP. Resistance to ONC201 was also induced in HCT116 cells by overexpression of D190A ClpP (FIG. 14E). Of note, over-expression of wild-type ClpP in parental Z138 and OCI-AML3 cell lines increased sensitivity to ONC201 and ONC212 (FIGS. 4H and 14D). Thus, these data indicate that activation of ClpP is functionally important for cell death induced by ONC201 and ONC212 and identify a mechanism of resistance to ClpP activators.

### Example 8—ClpP Activation Leads to Reduction in Respiratory Chain Complex Subunits and Impaired Oxidative Phosphorylation

Next, BioTD (Roux et al., 2012) was used to identify interacting partners of ClpP after chemical or genetic activation. To chemically hyperactivate the protease, T-REx HEK293 cells expressing FlagBirA-ClpP (WT) were treated with 0.6 μM ONC201 for 48 hours. As a genetic approach, FlagBirA-ClpP (Y118A) were expressed. The interactome of activated ClpP were compared to non-stimulated WT ClpP. Proteins that interacted with unstimulated WT ClpP in the BioID assay, but whose spectral counts decreased when ClpP was activated were postulated to represent potential substrates of hyperactivated ClpP.

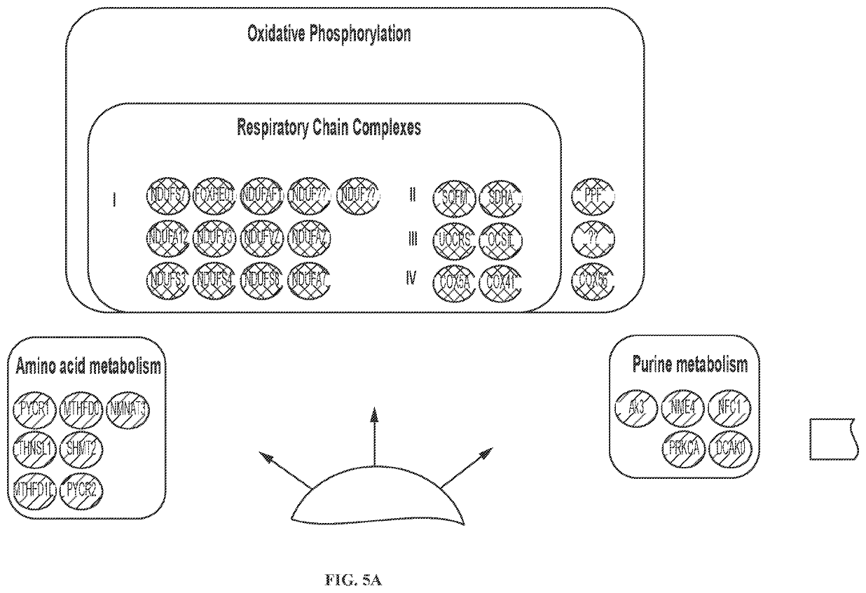

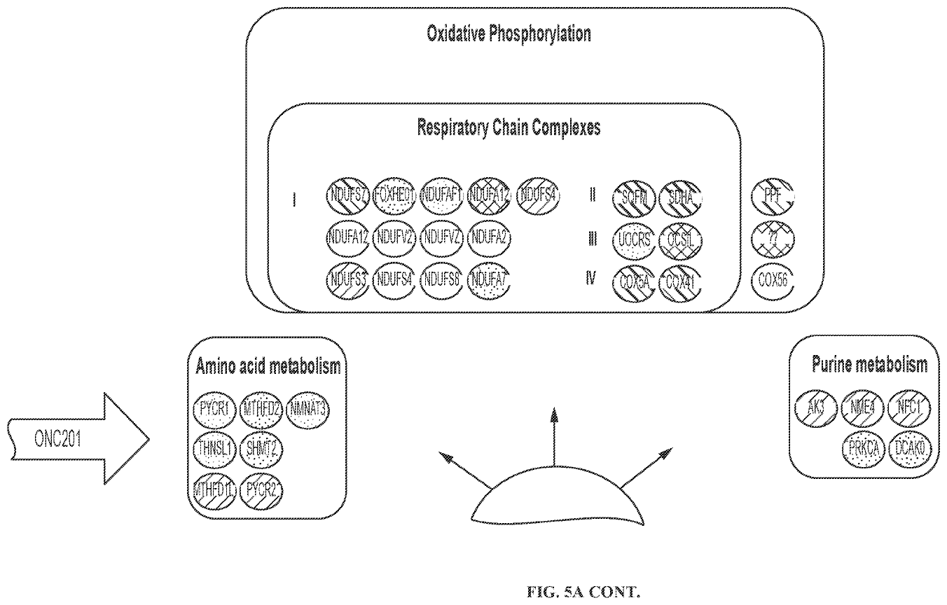

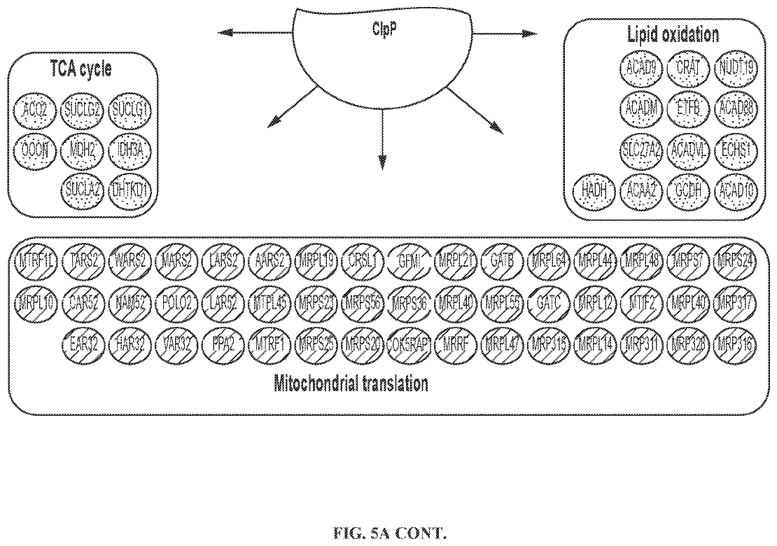

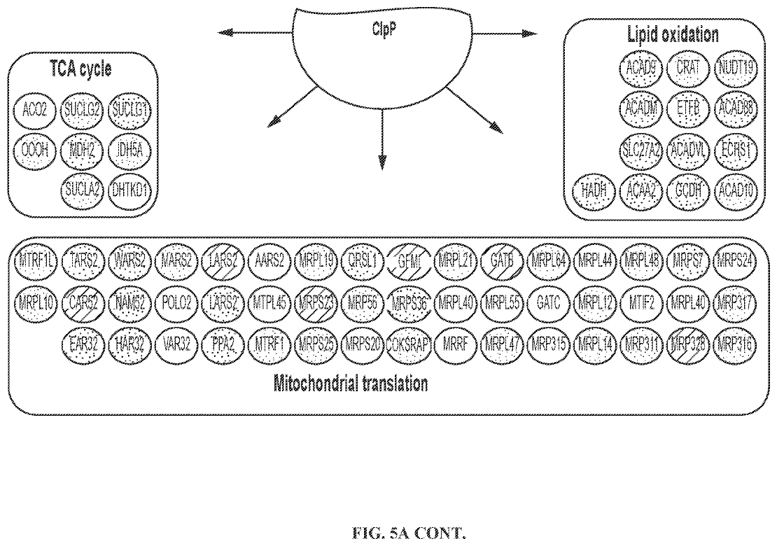

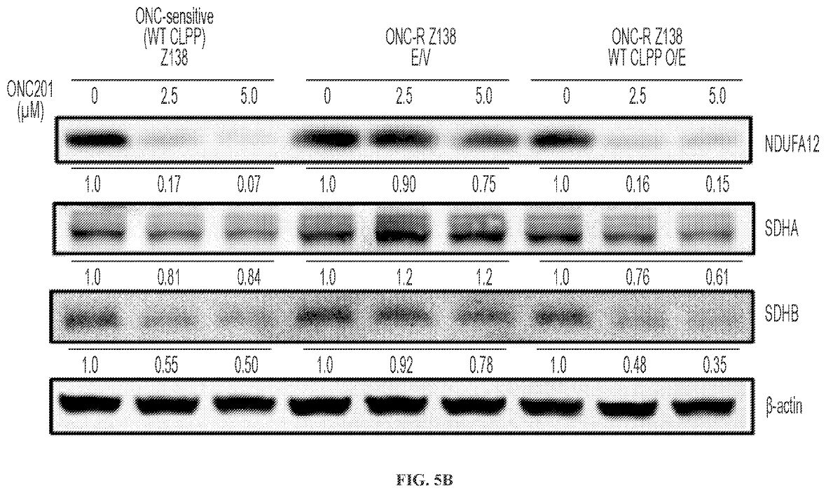

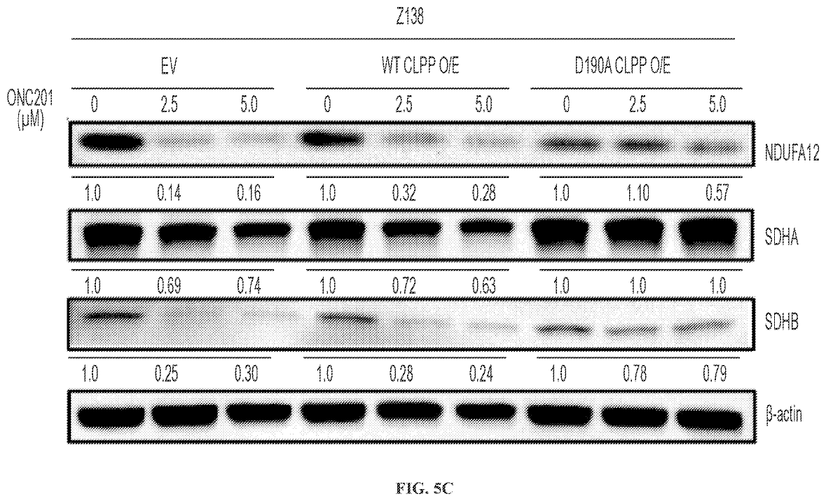

Over 200 mitochondrial proteins were identified as high confidence ClpP interacting partners. Of these polypeptides, 90 displayed a ≥4-fold decrease in spectral counts (p≤0.001) following ONC201 treatment. Amongst the proteins displaying the most robust decrease were components of the electron transport chain (and in particular, subunits of respiratory chain complex I) and polypeptides involved in mitochondrial translation (FIG. 5A & Table 1). Expression of the constitutively active Y118A ClpP mutant yielded a depletion of an overlapping set of interacting partners, but the degree of reduction in peptide counts was smaller than that observed in response to ONC201, likely reflecting the weaker activation of the protease by the mutation (Table 1).

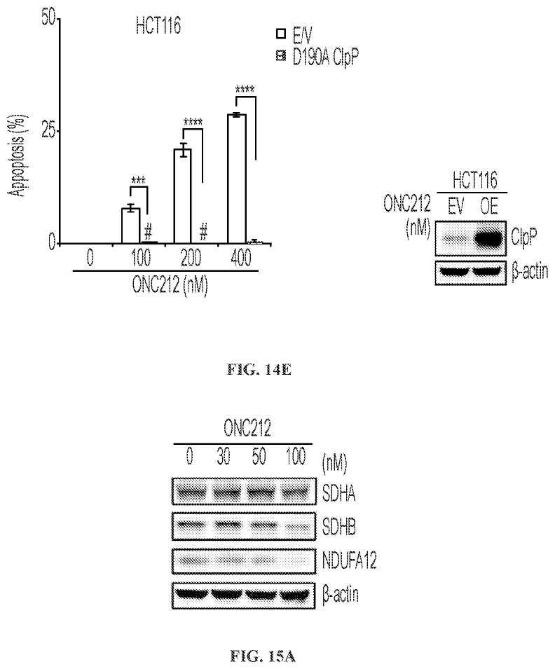

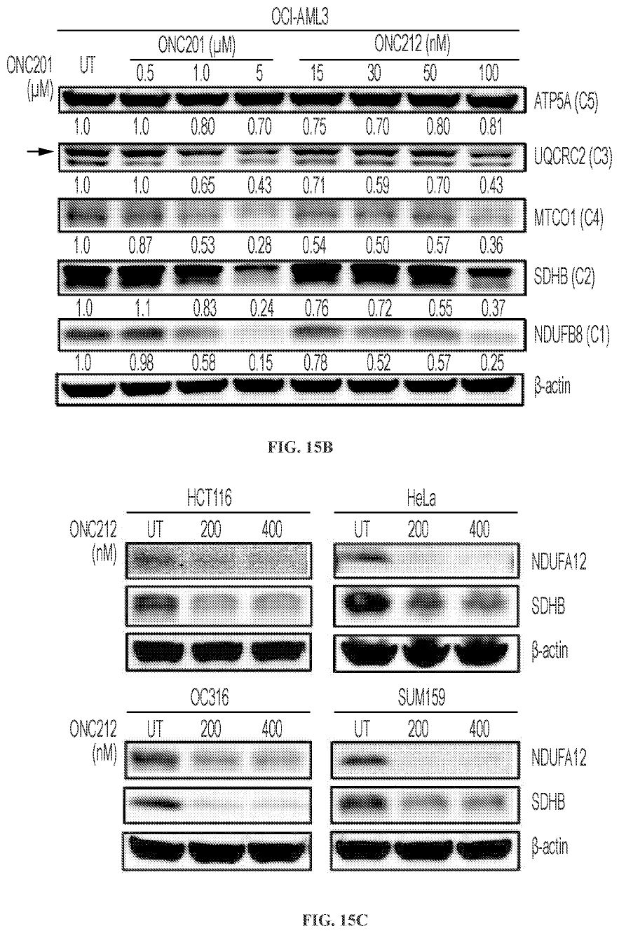

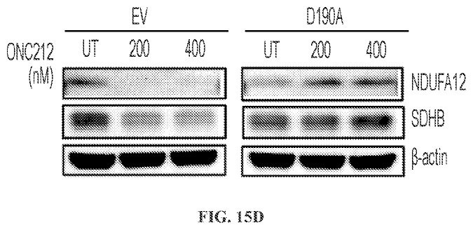

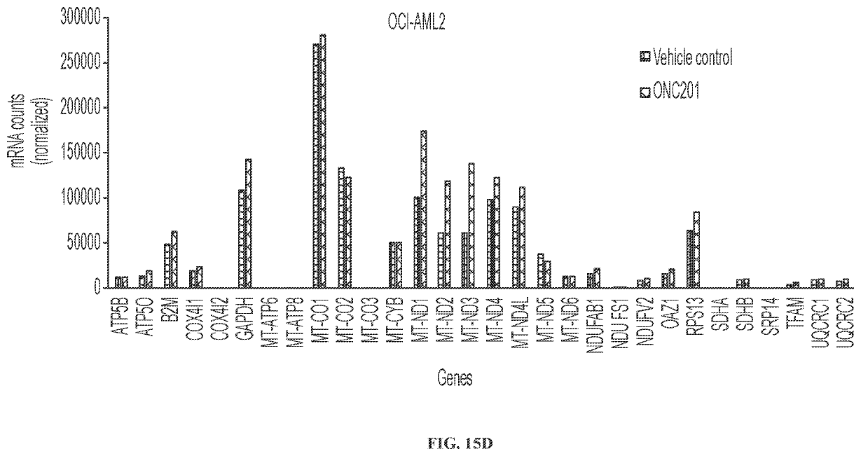

Previously, respiratory chain subunits SDHA and SDHB were identified as putative ClpP substrates and inhibition of ClpP led to the accumulation of degraded or misfolded subunits (Cole et al., 2015). To investigate the effects of ClpP activation on the levels of protein identified as interacting partners in the BioID assay, Z138 were treated with increasing concentrations of ONC201. Treatment with ONC201 decreased levels of respiratory chain complex proteins, such as SDHA and SDHB, and reductions in respiratory chain I subunits were most pronounced (FIG. 5B). In contrast, levels of these proteins did not significantly change after treating resistant Z138 carrying D190A mutant ClpP with ONC201 (FIG. 5B). Over-expressing wild-type ClpP in these cells restored sensitivity to ONC201 with depletion of respiratory chain complex proteins (FIG. 5B). Finally, over-expressing D190A ClpP mutant protein in wild type Z138 cells rendered them resistant to ONC201 with no significant reductions in respiratory chain proteins (FIG. 5C). ONC212 also reduced the level of the identified ClpP interactors in a dose dependent manner (FIGS. 15A, 15B). The reduction in NDUFA12 and SDHB by ONC212 was also observed in HCT116, HeLa, OC316, and SUM159 cells (FIG. 15C). The reduction of NDUFA12 and SDHB in HCT116 cells was blocked by overexpression of the inactivating mutant D190A ClpP (FIG. 15D), as observed in Z138 cells (FIG. 5C). While reductions in respiratory chain proteins were observed, levels of mRNA encoding mitochondrial respiratory chain substrates were either unchanged or increased (FIG. 16A). Furthermore, the addition of recombinant ClpP and ONC201 to lysates of mitochondria isolated from ClpP−/− HEK293T-REx and OCI-AML2 cells decreased levels of the complex I subunit NDUFB8 and complex III subunit UQCRC2, indicating that ClpP activation can increase the degradation of selective ClpP substrates, independent of cytoplasmic or nuclear pathways (FIG. 16B).

Likewise, the effects of induction of the Y118A ClpP activating mutant on respiratory chain subunits were examined in Z138 cells. Similar to the results with the chemical ClpP activators, induction of the Y118A ClpP mutant led to reductions in SDHA, SDHB, and NDUFA12 in a dose-dependent manner (FIG. 5D). In contrast, another respiratory complex subunit, ATP5A, was not reduced by Y118A ClpP overexpression, ONC201, or ONC212 (FIGS. 5D and S8B), reflecting selective degradation of particular subunits by ClpP activation.

The effects of ONC201 and ONC212 were also tested on levels of respiratory chain proteins in primary AML cells. Similar to the effects on cell lines, a reduction in respiratory chain proteins were observed in primary cells treated with the imipridones (FIG. 5E). Interestingly, similar reductions in respiratory chain proteins were also observed in normal hematopoietic cells (FIG. 5E). Thus, greater sensitivity of AML cells to ClpP activation likely reflects their increased reliance on oxidative phosphorylation and lower spare reserve capacity in their respiratory chain (Sriskanthadevan et al., 2015).

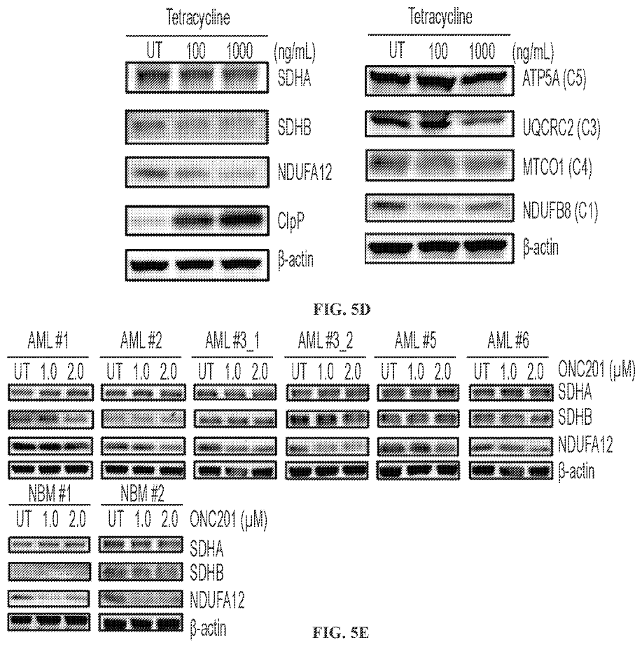

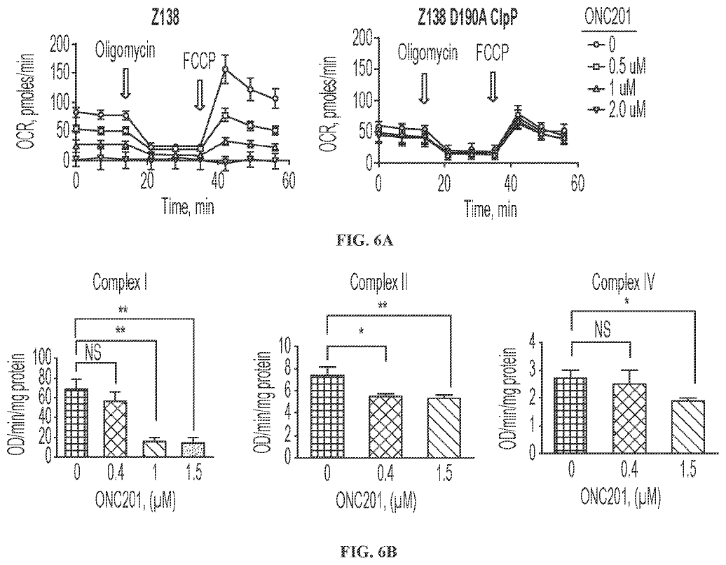

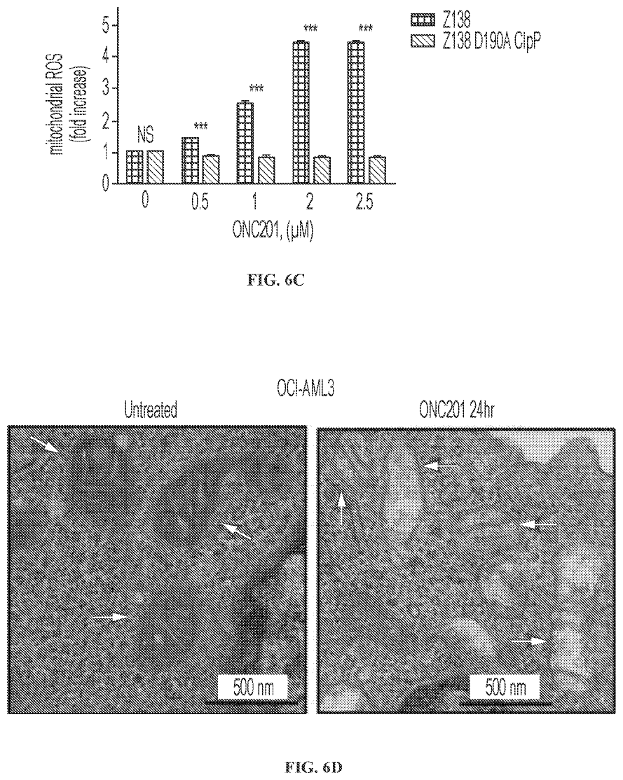

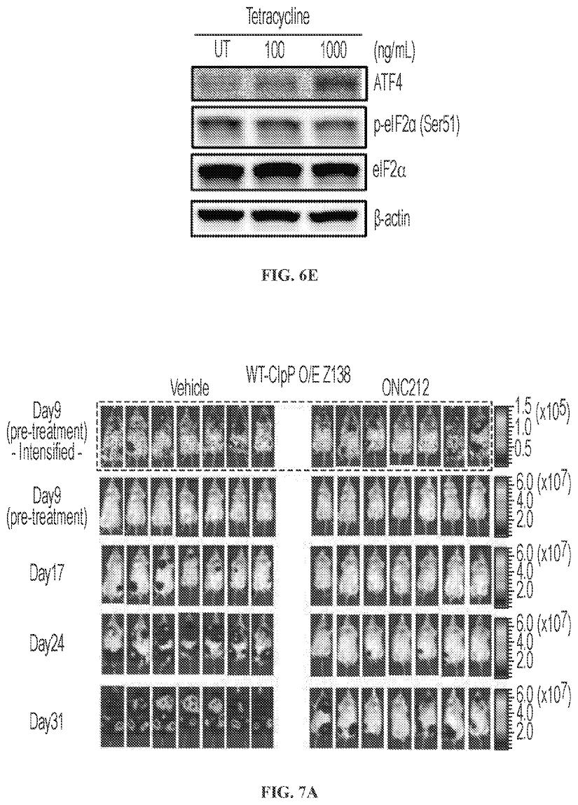

Next, the effects of ClpP activation on oxidative phosphorylation and mitochondrial function were investigated. Z138 cells carrying WT and D190A ClpP were treated with increasing concentrations of ONC201. Treatment with ONC201 decreased basal OCR and spare reserve capacity in Z138 cells with WT ClpP, while no change was observed in Z138 cells with D190A ClpP (FIG. 6A). Likewise, ClpP activation decreased the enzymatic activity of respiratory chain complexes I, II, and IV, with complex I being the most sensitive (FIG. 6B). Finally, ClpP activation increased the production of mitochondrial ROS in Z138 cells with WT ClpP, but no change was seen in Z138 cells with D190A ClpP (FIG. 6C). Consistently, mitochondria were morphologically damaged by ONC201 treatment, as assessed by electron microscopy, demonstrating particular damages of matrix and cristae structures (FIG. 6D).

ONC201 induces atypical integrated stress response (ISR) where ATF4 protein increase is induced irrespectively of phosphorylation status of eIF2α, unlike classical ISRs (Ishizawa et al., 2016). Indeed, overexpression of Y118A ClpP in Z138 cells showed increase in ATF4 protein without increasing phosphorylation of eIF2α (FIG. 6E).

### Example 9—ClpP Activation by Imipridones Exerts Anti-Tumor Effects In Vivo

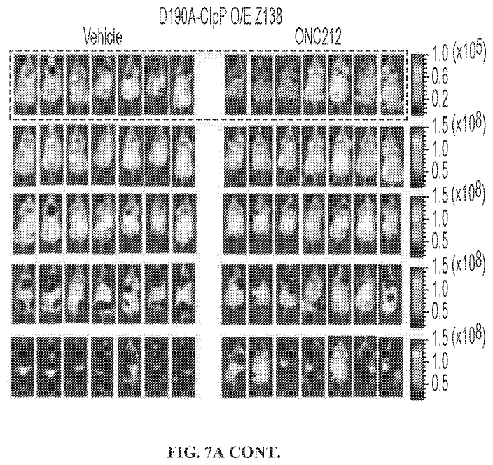

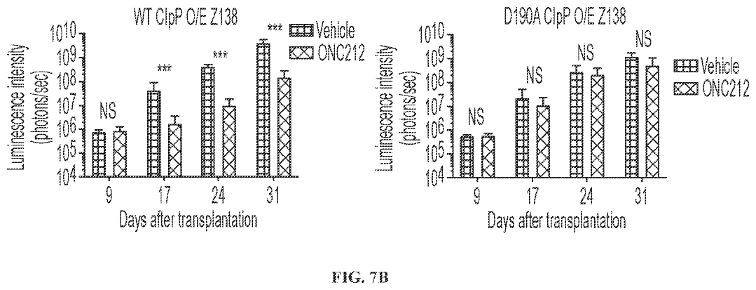

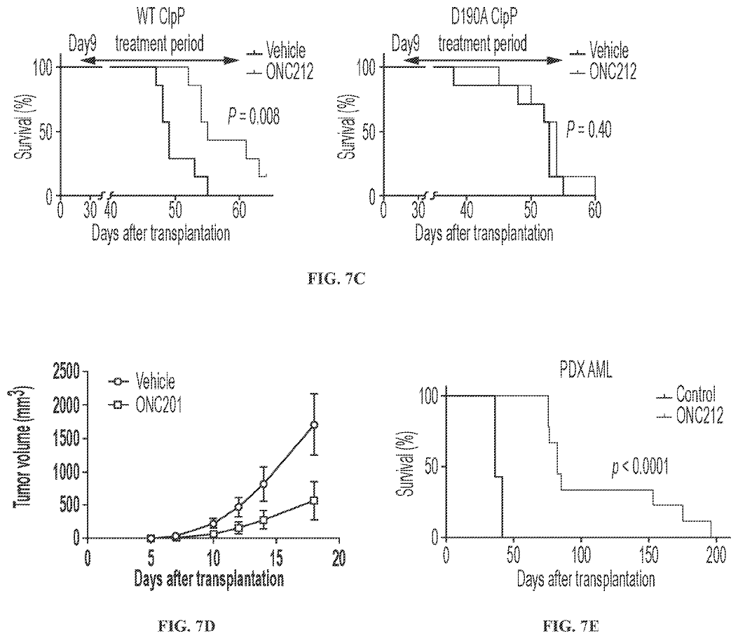

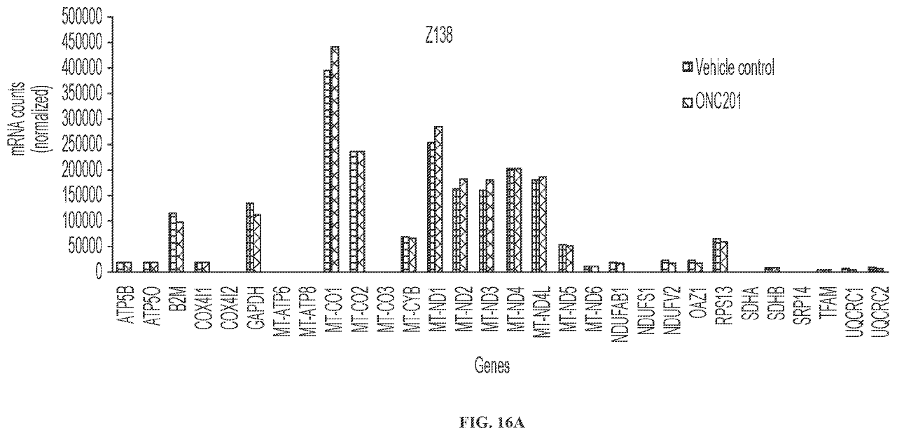

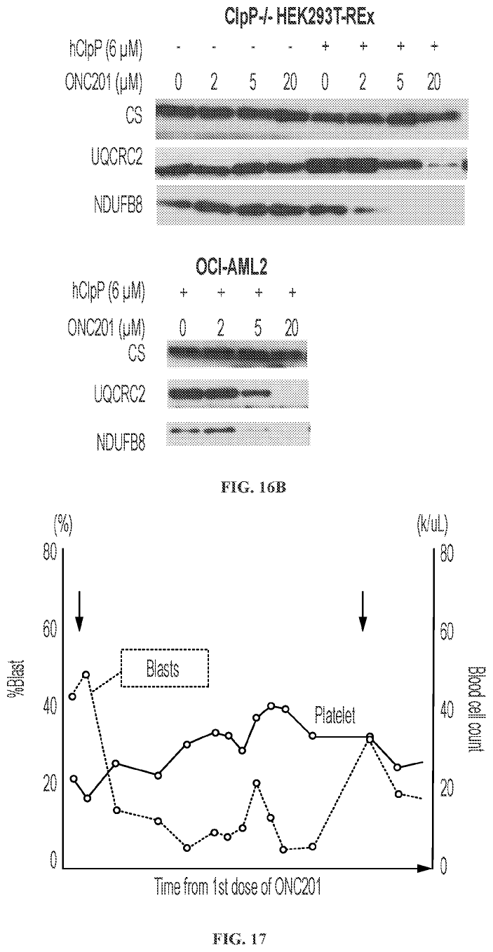

To test if ClpP activation by ONC212 induces anti-tumor effects in vivo, xenograft mouse models were established using Z138 cells with WT or D190A ClpP overexpression, and the mice were treated with oral gavage of ONC212. The Z138 cells were luciferase-labeled, and systemic tumor burden was followed by measuring luciferase activity with IVIS imaging. Consistent with the in vitro findings, tumor burden was significantly reduced by ONC212 treatment in the WT ClpP group, whereas there was no discernable anti-tumor activity in the D190A ClpP group (FIGS. 7A, 7B). The resultant survival was significantly prolonged in the ONC212-treated WT ClpP group, but not in the D190A mutant group (median survival: WT; 49 vs 55 days, p=0.008, D190A mutant; 53 vs 54 days, p=0.40) (FIG. 7C). The results indicated that in vivo efficacy of ONC212 is ClpP-dependent. The in vivo anti-tumor effects of ONC201 were also validated in a xenograft model of OCI-AML2 cells. Oral ONC201 significantly reduced the leukemic burden in mice compared to the control group (FIG. 7D). Collectively, imipridones are effective in vivo in lymphoma and AML mouse models. To further evaluate the effects of ONC212 on leukemia-initiating cells (LICs), patient-derived xenograft AML cells from secondarily engrafted mice (i.e., LICs enriched) were treated with ONC212 and then the cells were injected into recipient NSG mice. Survival of the mice was significantly prolonged (median survival: 36 vs 82 days, p<0.0001) (FIG. 7E), suggesting that ClpP activation inhibits the engraftment capacity of LICs. In an ongoing clinical trial in patients with relapsed/refractory AML, decrease in circulating blasts and subsequent increase in platelet counts was observed (FIG. 17) following a single dose of ONC201 (250 mg orally).

### Example 10—Genetic Activation of ClpP Sensitizes Leukemia and Lymphoma Cells to Venetoclax (ABT-199)

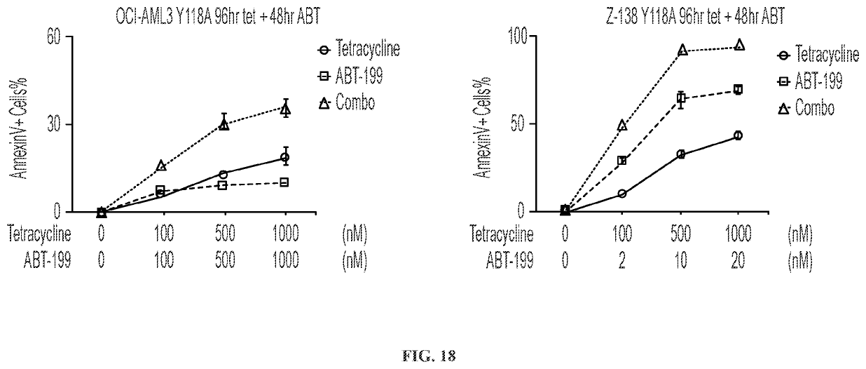

Constitutively active ClpP mutant (Y118A), with the tetracycline-inducible system, was transfected by lentivirus into OCI-AML3 and Z138 cells. Cells were treated with tetracycline, which induces Y118A ClpP mutant in a tetracycline dose-dependent manner by 72 hrs, and subsequently exposed to venetoclax (ABT-199) at the indicated concentrations (FIG. 18). Genetic activation of ClpP sensitized the cells to venetoclax, which is consistent with the synergy in combination treatment of ONC201 and venetoclax, indicating the significance of ClpP activity in inducing synergistic cancer cell killing in the combination therapy.

### Example 11—Responders in ONC201 Clinical Trials Showed ClpP-Positive Leukemia Cells, while a Non-Responder was Negative for ClpP

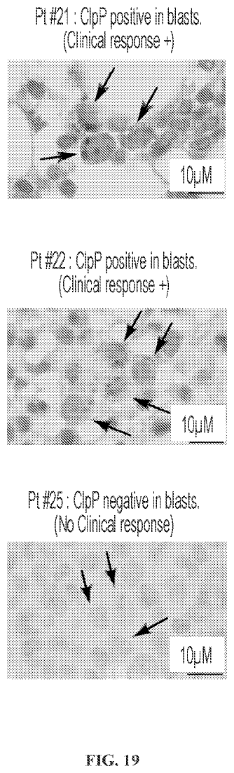

Pre-treatment bone marrow biopsy samples were obtained from 11 patients among the 30 enrolled patients, and stained for ClpP. Representative micrographs are shown in FIG. 19. Blasts in Patient #21 and #22 were positive for ClpP; perinuclear staining was consistent with mitochondrial localization. This finding is consistent with the clinical responses observed in these patients during ONC201 treatment. On the other hand, blasts from Patient #25, who did not achieve a clinical response, were negative for ClpP.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Adams et al., “PHENIX: a comprehensive Python-based system for macromolecular structure solution,” Acta Crystallographica Section D, Biological Crystallography, 66:213-221, 2010.
Afonine & Adams, “On the contribution of hydrogen atoms to X-ray scattering,” Computational Crystallography Newsletter, 3:18-21, 2012.
Allen et al., “Discovery and clinical introduction of first-in-class imipridone ONC201,” Oncotarget, 7:74380-74392, 2016.
Allen et al., “Dual inactivation of Akt and ERK by TIC10 signals Foxo3a nuclear translocation, TRAIL gene induction, and potent antitumor effects,” Science Translational Medicine, 5:171ra117, 2013.
Arrillaga-Romany et al., “A phase 2 study of the first imipridone ONC201, a selective DRD2 antagonist for oncology, administered every three weeks in recurrent glioblastoma,” Oncotarget, 8:79298-79304, 2017.
Bezawork-Geleta et al., “LON is the master protease that protects against protein aggregation in human mitochondria through direct degradation of misfolded proteins,” Scientifc Reports, 5:17397, 2015.
Birsoy et al., “Metabolic determinants of cancer cell sensitivity to glucose limitation and biguanides,” Nature, 508:108-112, 2014.
Brotz-Oesterhelt et al., “Dysregulation of bacterial proteolytic machinery by a new class of antibiotics,” Nature Medicine, 11:1082-1087, 2005.
Cole et al., “Inhibition of the Mitochondrial Protease ClpP as a Therapeutic Strategy for Human Acute Myeloid Leukemia,” Cancer Cell, 27:864-876, 2015.
Conlon et al., “Activated ClpP kills persisters and eradicates a chronic biofilm infection.,” Nature, 503:365-370, 2013.
Corydon et al., “A human homologue of Escherichia coli ClpP caseinolytic protease: recombinant expression, intracellular processing and subcellular localization,” The Biochemical Journal, 331 (Pt 1):309-316, 1998.
Craig & Beavis, “TANDEM: matching proteins with tandem mass spectra,” Bioinform, 20:1466-1467, 2004.
de Sagarra et al., “Mitochondrial localization and oligomeric structure of HClpP, the human homologue of E. coli ClpP,” Journal of Molecular Biology, 292:819-825, 1999.
DeLano, The PyMOL. Mol Graph Syst, DeLano Scientific, Palo Alto, 2002.
Emsley & Cowtan, “Coot: model-building tools for molecular graphics,” Acta Crystallographica Section D, Biological Crystallography, 60:2126-2132, 2004.
Emsley et al., “Features and development of Coot,” Acta Crystallographica Section D, Biological Crystallography, 66:486-501, 2010.
Eng et al., “Comet: an open-source MS/MS sequence database search tool,” Proteomics, 13:22-24, 2013.
Farge et al., “Chemotherapy-Resistant Human Acute Myeloid Leukemia Cells Are Not Enriched for Leukemic Stem Cells but Require Oxidative Metabolism,” Cancer Discovery, 7:716-735, 2017.
Frolova et al., “Regulation of HIF-1alpha signaling and chemoresistance in acute lymphocytic leukemia under hypoxic conditions of the bone marrow microenvironment,” Cancer Biology & Therapy, 13:858-870, 2012.
Gersch et al., “AAA+ chaperones and acyldepsipeptides activate the ClpP protease via conformational control,” Nature Communications, 6:6320, 2015.
Gersch et al., “Barrel-shaped ClpP Proteases Display Attenuated Cleavage Specificities,” ACS Chemical Biology, 11:389-399, 2016.
Greer et al., “ONC201 kills breast cancer cells in vitro by targeting mitochondria,” Oncotarget, 9:18454-18479, 2018.
Haynes et al., “ClpP mediates activation of a mitochondrial unfolded protein response in C. elegans,” Developmental Cell, 13:467-480, 2007.
Houtkooper et al., “Mitonuclear protein imbalance as a conserved longevity mechanism,” Nature, 497:451-457, 2013.
Ishizawa et al., “ATF4 induction through an atypical integrated stress response to ONC201 triggers p53-independent apoptosis in hematological malignancies,” Science Signaling, 9:ra17, 2016.
Jafari et al., “The cellular thermal shift assay for evaluating drug target interactions in cells,” Nature Protocols, 9:2100-2122, 2014.
Jin & Youle, “The accumulation of misfolded proteins in the mitochondrial matrix is sensed by PINK1 to induce PARK2/Parkin-mediated mitophagy of polarized mitochondria,” Autophagy, 9:1750-1757, 2013.
Kabsch, “XDS.” Acta Crystallographica Section D, Biological Crystallography, 66:125-132, 2010.
Kang et al., “Human mitochondrial ClpP is a stable heptamer that assembles into a tetradecamer in the presence of ClpX,” The Journal of Biological Chemistry, 280:35424-35432, 2005.
Kang et al., “Crystallography and mutagenesis point to an essential role for the N-terminus of human mitochondrial ClpP,” Journal of Structural Biology, 148:338-352, 2004.
Kessner et al., “ProteoWizard: open source software for rapid proteomics tools development,” Bioinformatics, 24:2534-2536, 2008.
Kimber et al., ‘Structural and theoretical studies indicate that the cylindrical protease ClpP samples extended and compact conformations,” Structure, 18:798-808, 2010.
Kline et al., “ONC201 kills solid tumor cells by triggering an integrated stress response dependent on ATF4 activation by specific eIF2alpha kinases,” Science Signaling, 9:ra18, 2016.
Kline et al., “Role of Dopamine Receptors in the Anticancer Activity of ONC201,” Neoplasia, 20:80-91, 2018.
Konopleva et al., “Mechanisms of apoptosis sensitivity and resistance to the BH3 mimetic ABT-737 in acute myeloid leukemia,” Cancer Cell, 10:375-388, 2006.
Konopleva et al., “Efficacy and Biological Correlates of Response in a Phase II Study of Venetoclax Monotherapy in Patients with Acute Myelogenous Leukemia,” Cancer Discovery, 6:1106-1117, 2016.
Kotschy et al., “The MCL1 inhibitor S63845 is tolerable and effective in diverse cancer models,” Nature, 538:477-482, 2016.
Kuntz et al., “Targeting mitochondrial oxidative phosphorylation eradicates therapy-resistant chronic myeloid leukemia stem cells,” Nature Medicine, 23:1234-1240, 2017.
Lagadinou et al., “BCL-2 inhibition targets oxidative phosphorylation and selectively eradicates quiescent human leukemia stem cells,” Cell Stem Cell, 12:329-341, 2013.
Lee et al., “Structures of ClpP in complex with acyldepsipeptide antibiotics reveal its activation mechanism,” Nature Structural & Molecular Biology, 17:471-478, 2010.
Lee et al., “An improved SUMO fusion protein system for effective production of native proteins,” Protein Science, 17:1241-1248, 2008.
Leung et al., “Activators of cylindrical proteases as antimicrobials: identification and development of small molecule activators of ClpP protease,” Chemistry & Biology, 18:1167-1178, 2011.
Lev et al., “Anti-pancreatic cancer activity of ONC212 involves the unfolded protein response (UPR) and is reduced by IGF1-R and GRP78/BIP,” Oncotarget, 8:81776-81793, 2017.
Marin-Valencia et al., “Analysis of tumor metabolism reveals mitochondrial glucose oxidation in genetically diverse human glioblastomas in the mouse brain in vivo,” Cell Metabolism, 15:827-837, 2012.
McCoy et al., “Phaser crystallographic software,” Journal of Applied Crystallography, 40:658-674, 2007.
Moschoi et al., “Protective mitochondrial transfer from bone marrow stromal cells to acute myeloid leukemic cells during chemotherapy,” Blood, 128:253-264, 2016.
Muller et al., “Fluorine in pharmaceuticals: looking beyond intuition,” Science, 317:1881-1886, 2007.
Munch & Harper, “Mitochondrial unfolded protein response controls matrix pre-RNA processing and translation,” Nature, 534:710-713, 2016.
Ni et al., “Characterization of Gain-of-Function Mutant Provides New Insights into ClpP Structure,” ACS Chemical Biology, 11:1964-1972, 2016.
Pan et al., “Selective BCL-2 inhibition by ABT-199 causes on-target cell death in acute myeloid leukemia,” Cancer Discovery, 4:362-375, 2014.
Pan et al., “Synthetic Lethality of Combined Bcl-2 Inhibition and p53 Activation in AML: Mechanisms and Superior Antileukemic Efficacy,” Cancer Cell, 32:748-760 e746, 2017.
Quiros et al., “Mitonuclear communication in homeostasis and stress,” Nature Reviews: Molecular Cell Biology, 17:213-226, 2016.
Roux et al., “A promiscuous biotin ligase fusion protein identifies proximal and interacting proteins in mammalian cells,” The Journal of Cell Biology, 196:801-810, 2012.
Samudio et al., “Pharmacologic inhibition of fatty acid oxidation sensitizes human leukemia cells to apoptosis induction,” The Journal of Clinical Investigation, 120:142-156, 2010.
Seiferling et al., “Loss of CLPP alleviates mitochondrial cardiomyopathy without affecting the mammalian UPRmt,” EMBO Reports, 17:953-964, 2016.
Skrtic et al., “Inhibition of mitochondrial translation as a therapeutic strategy for human acute myeloid leukemia,” Cancer Cell, 20:674-688, 2011.
Sprangers et al., “Quantitative NMR spectroscopy of supramolecular complexes: dynamic side pores in ClpP are important for product release,” Proc. Natl. Acad. Sci. U.S.A., 102:16678-16683, 2005.
Sriskanthadevan et al., “AML cells have low spare reserve capacity in their respiratory chain that renders them susceptible to oxidative metabolic stress,” Blood, 125:2120-2130, 2015.
Stein et al., “First-in-Human Clinical Trial of Oral ONC201 in Patients with Refractory Solid Tumors,” Clinical Cancer Research, 23:4163-4169, 2017.
Tu et al., “The Imipridone ONC201 Induces Apoptosis and Overcomes Chemotherapy Resistance by Up-Regulation of Bim in Multiple Myeloma,” Neoplasia, 19:772-780, 2017.
Viale et al., “Oncogene ablation-resistant pancreatic cancer cells depend on mitochondrial function,” Nature, 514:628-632, 2014.
Wagner et al., “Preclinical evaluation of the imipridone family, analogs of clinical stage anti-cancer small molecule ONC201, reveals potent anti-cancer effects of ONC212,” Cell Cycle, 16:1790-1799, 2017.
Wang et al., “ANNOVAR: functional annotation of genetic variants from high-throughput sequencing data,” Nucleic Acids Research, 38:e164, 2010.
Warner et al., “Direct evidence for cooperating genetic events in the leukemic transformation of normal human hematopoietic cells,” Leukemia, 19:1794-1805, 2005.
Winn et al., “Overview of the CCP4 suite and current developments,” Acta Crystallographica Section D, Biological Crystallography, 67:235-242, 2011.
Wong et al., “Acyldepsipeptide analogs dysregulate human mitochondrial ClpP protease activity and cause apoptotic cell death,” Cell Chem. Biol., 25:1017-1030, 2018.
Zhao et al., “A mitochondrial specific stress response in mammalian cells,” The EMBO Journal, 21:4411-4419, 2002.

What is claimed is:

1. A method of selecting a patient having a cancer for treatment with an agent that activates mitochondrial proteolysis, the method comprising (a) determining a ClpP level in the cancer, and (b) selecting the patient for treatment if the ClpP level in the cancer is higher than a reference level.

2. The method of claim 1, wherein the reference level is a level that is one standard deviation below an average ClpP level in a healthy population.

3. The method of any one of claims 1-2, further comprising administering an effective amount of an agent that activates mitochondrial proteolysis.

4. The method of claim 3, wherein the agent that activates mitochondrial proteolysis is a ClpP activating agent.

5. The method of claim 4, wherein the ClpP activating agent is an imipridone.

6. The method of claim 5, wherein the imipridone is ONC201, ONC206, ONC212, or ONC213.

7. A method of treating a patient having a cancer, the method comprising administering a therapeutically effective amount of an agent that activates mitochondrial proteolysis to the patient, wherein the patient's cancer has a ClpP level that is higher than a reference level.

8. A method of treating a patient having a cancer, the method comprising:
(a) detecting whether the patient's cancer has a ClpP level that is higher than a reference level by:
(i) obtaining or having obtained a biological sample from the cancer; and
(ii) performing or having performed an assay on the biological sample to determine a ClpP level;
(b) selecting or having selected the patient for treatment when the cancer has a ClpP level that is higher than a reference level; and
(c) administering or having administered to the selected patient a therapeutically effective amount of an agent that activates mitochondrial proteolysis.

9. The method of claim 7 or 8, wherein the reference level is a level that is one standard deviation below an average ClpP level in a healthy population.

10. The method of any one of claims 1-9, wherein the ClpP level in the cancer is determined by western blot, ELISA, immunoassay, radioimmunoassay, or mass spectrometry.

11. The method of any one of claims 3-10, further comprising administering at least a second anti-cancer therapy to the patient.

12. The method of claim 11, wherein the second anti-cancer therapy is a surgical therapy, chemotherapy, radiation therapy, cryotherapy, hormonal therapy, toxin therapy, immunotherapy, or cytokine therapy.

13. The method of claim 12, wherein the chemotherapy is venetoclax.

14. The method of claim 12, wherein the immunotherapy is an immune checkpoint inhibitor.

15. The method of any one of claims 1-14, further comprising reporting the ClpP level.

16. The method of claim 15, wherein the reporting comprises preparing a written or electronic report.

17. The method of claim 16, further comprising providing the report to the subject, a doctor, a hospital, or an insurance company.

18. A method of selecting a patient having a cancer for treatment with an agent that activates mitochondrial proteolysis, the method comprising (a) determining a ClpP protein mutation status in the cancer, and (b) selecting the patient for treatment if the cancer has a D190A mutation in the ClpP protein.

19. The method of claim 18, further comprising administering an effective amount of an agent that activates mitochondrial proteolysis.

20. The method of claim 19, wherein the agent that activates mitochondrial proteolysis is a ClpP activating agent.

21. The method of claim 20, wherein the ClpP activating agent is an imipridone.

22. The method of claim 21, wherein the imipridone is ONC201, ONC206, ONC212, or ONC213.

23. A method of treating a patient having a cancer, the method comprising administering a therapeutically effective amount of an agent that activates mitochondrial proteolysis to the patient, wherein the patient's cancer has a D190A mutation in a ClpP protein.

24. A method of treating a patient having a cancer, the method comprising:
(a) detecting whether the patient's cancer has a D190A mutation in a ClpP protein by:
(i) obtaining or having obtained a biological sample from the cancer; and
(ii) performing or having performed an assay on the biological sample to determine whether the patient's cancer has a D190A mutation in a ClpP protein;
(b) selecting or having selected the patient for treatment when the cancer has a D190A mutation in the ClpP protein; and
(c) administering or having administered to the selected patient a therapeutically effective amount of an agent that activates mitochondrial proteolysis.

25. The method of any one of claims 18-24, wherein the D190A mutation in the ClpP protein is detected by western blot, ELISA, mass spectrometry, or sequencing a nucleic acid encoding ClpP.

26. The method of claim 25, wherein the western blot or ELISA are performed using an antibody that specifically detects ClpP having the D190A mutation.

27. The method of claim 25, wherein the nucleic acid is an mRNA encoding ClpP.

28. The method of claim 25, wherein the nucleic acid is genomic DNA encoding ClpP.

29. The method of any one of claims 19-28, wherein the agent that activates mitochondrial proteolysis is a ClpP activating agent.

30. The method of claim 29, wherein the ClpP activating agent is an imipridone.

31. The method of claim 30, wherein the imipridone is ONC201, ONC206, ONC212, or ONC213.

32. The method of any one of claims 19-31, further comprising administering at least a second anti-cancer therapy to the patient.

33. The method of claim 32, wherein the second anti-cancer therapy is a surgical therapy, chemotherapy, radiation therapy, cryotherapy, hormonal therapy, toxin therapy, immunotherapy, or cytokine therapy.

34. The method of claim 33, wherein the chemotherapy is venetoclax.

35. The method of claim 33, wherein the immunotherapy is an immune checkpoint inhibitor.

36. The method of any one of claims 18-35, further comprising reporting the ClpP D190A mutation status.

37. The method of claim 36, wherein the reporting comprises preparing a written or electronic report.

38. The method of claim 37, further comprising providing the report to the subject, a doctor, a hospital, or an insurance company.

39. The method of any one of claims 1-38, wherein the patient is in remission and the method prevents relapse.

40. The method of any one of claims 1-39, wherein the method eliminates chemo-resistant cells.

41. The method of any one of claims 1-40, wherein the cancer is AML.

42. The method of any one of claims 1-41, wherein the patient has previously undergone at least one round of anti-cancer therapy.

43. The method of any one of claims 1-42, wherein the patient is a human.

44. A method of killing bacterial cells, the method comprising contacting the bacterial cells with a lethal amount an imipridone.

45. The method of claim 44, wherein the bacterium is a gram-positive bacterium.

46. The method of claim 44, wherein the bacterium is selected from the group consisting of Staphylococcus, Streptococcus, Enterococcus, Clostridium, Corynebacterium, and Peptostreptococcus.

47. The method of claim 46, wherein the bacterium is Staphylococcus.

48. A method of treating a bacterial infection in a subject in need thereof, the method comprising administering to the subject a therapeutically effective amount of an imipridone.

49. The method of claim 48, wherein the bacteria are antibiotic resistant.

50. The method of claim 48 or 49, wherein the bacterium is a gram-positive bacterium.

51. The method of claim 48 or 49, wherein the bacterium is selected from the group consisting of Staphylococcus, Streptococcus, Enterococcus, Clostridium, Corynebacterium, and Peptostreptococcus.

52. The method of claim 51, wherein the bacterium is Staphylococcus.

53. A method of treating a patient having Perrault syndrome, the method comprising administering or having administered to the selected patient a therapeutically effective amount of an agent that activates mitochondrial proteolysis.

54. The method of claim 53, wherein the agent that activates mitochondrial proteolysis is a ClpP activating agent.

55. The method of claim 54, wherein the ClpP activating agent is an imipridone.

56. The method of claim 55, wherein the imipridone is ONC201, ONC206, ONC212, ONC213.

57. The method of any one of claims 52-56, where the patient has a mutation in CLPP or HSDI7B4.

58. The method of any one of claims 52-57, wherein the method improves the patient's hearing, prevents further hearing loss in the patient, or prevents hearing loss from occurring in the patient.

59. The method of any one of claims 52-58, wherein the patient is female, wherein the method improves ovarian function in the patient, prevents further ovarian dysgenesis in the patient, or prevents ovarian dysgenesis from occurring in the patient.
